(19) United States
(12) Patent Application Publication
(10) Pub. No.: US 2022/0048876 A1
(43) Pub. Date: Feb. 17, 2022

(54) INSECTICIDAL COMPOUNDS

(71) Applicant: SYNGENTA PARTICIPATIONS AG, Basel (CH)

(72) Inventors: Jérôme Yves CASSAYRE, Stein (CH); Myriem EL QACEMI, Stein (CH); Thomas PITTERNA, Stein (CH); André STOLLER, Stein (CH)

(73) Assignee: SYNGENTA PARTICIPATIONS AG, Basel (CH)

(21) Appl. No.: 17/279,959

(22) PCT Filed: Sep. 20, 2019
(86) PCT No.: PCT/EP2019/075388

§ 371 Date: Mar. 25, 2021

(30) Foreign Application Priority Data
Sep. 26, 2018 (EP) 18196951.0

(51) Int. Cl. C07D 307/28; C07D 275/02; C07D 261/04; A01N 43/80; A01N 43/06; A01N 43/36; C07D 307/30

(52) U.S. Cl. CPC C07D 307/28; C07D 275/02; C07D 261/04; C07D 307/30; A01N 43/06; A01N 43/36; A01N 43/80

(57) ABSTRACT

Compounds of formula (I), wherein the substituents are as defined in claim 1, and the agrochemically acceptable salts salts, stereoisomers, enantiomers, tautomers and N-oxides of those compounds, and their uses as insecticides.

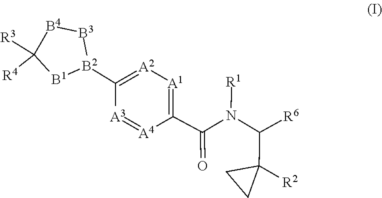

The present invention relates to certain isoxazoline derivatives, to intermediates for preparing such derivatives, to insecticidal, acaricidal, nematicidal and molluscicidal compositions comprising such derivatives and to methods of using these derivatives to control insect, acarine, nematode and mollusc pests.

Certain isoxazoline derivatives with insecticidal properties are disclosed, for example, in WO2011067272. It has now surprisingly been found that certain isoxazolidine, dihydrothiophene, dihydrolsothiazole, pyrrolidine and dihydrofurane derivatives have highly potent insecticidal properties.

The present invention accordingly relates to compounds of formula (I),

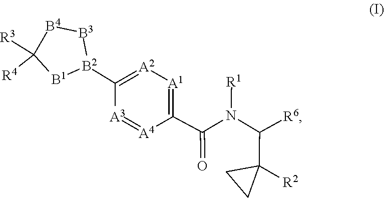

wherein
A¹, A², A³ and A⁴ are, independently of one another, C—H, C—R⁵ or N;
B¹—B²—B³—B⁴ is —C(R^{5a}R^{5b})—C═N—O—, —CH₂—C═N—CH₂—, —CH₂—C═CH₂—S—, —CH₂—C═N—S—, —CH₂—N—CH₂—CH₂—, —CH₂—C═CH—O—, —CH(OH)—N—CH₂—CH₂—, —C(O)—N—CH₂—CH₂- or —CH═C—CH₂—O—;
R¹ is hydrogen, C₁-C₈alkyl, C₁-C₈alkylcarbonyl-, C₁-C₈alkoxy, C₁-C₈alkoxy-C₁-C₈alkyl, C1-C₈alkoxycarbonyl, C₁-C₈alkylcarbonyloxyC₁-C₈alkyl, C₁-C₈alkoxycarbonylsulfanyl, C₁-C₈alkylaminocarbonyloxyC₁-C₈alkyl, C₁-C₈dialkylaminocarbonyloxyC₁-C₈alkyl, C₁-C₈alkylaminocarbonylC₁-C₈alkyl, C₁-C₈dialkylaminocarbonylC₁-C₈alkyl or C₁-C₈alkoxycarbonylC₁-C₈alkylaminoC₁-C₈alkyl, wherein each alkyl or alkoxy group may be optionally substituted with from one to three halogen atoms or with a cyano group; or
R₁ is selected from —(C₀-C₄alkyl)-C(═O)—C₃-C₆cycloalkyl, —(C₀-C₄alkyl)-O—C(═O)—C₃-C₆cycloalkyl, —(C₀-C₄alkyl)-O—C₃-C₆cycloalkyl, —R^{1a}OR^{1a}OR^{1b}, —R^{1a}OC(═O)R^{1b}, —R^{1a}OC(═O)OR^{1b}, —R^{1a}N(R^{1c})C(═O)OR^{1b}, —R^{1a}OC(═O)N(R^{1b})(R^{1c}), —R^{1a}C(═O)N(R^{1b})(R^{1c}) and —S—C(═O)OR^{1b};
R^{1a} is —(CR^{1d}R^{1e})ₙ;
R^{1b} and R^{1c} are independently selected from H and C₁-C₄alkyl, wherein each alkyl group is unsubstituted or substituted with one to three halogen atoms or with a cyano group;
R^{1d} and R^{1e} are independently selected from H and C₁-C₄alkyl;
n is selected from 1, 2, 3 and 4;
R² is cyano, C(S)NH₂ or C(O)NH₂;
R³ is C₁-C₈haloalkyl;
R⁴ is aryl, aryl substituted by one to three R⁷, heteroaryl or heteroaryl substituted by one to three R⁷;
R⁵ is independently halogen, cyano, nitro, C₁-C₈alkyl, C₃-C₈cycloalkyl, C₁-C₈haloalkyl, C₂-C₈alkenyl, C₂-C₈haloalkenyl, C₂-C₈alkynyl, C₂-C₈haloalkynyl, C₁-C₈alkoxy, C₁-C₈haloalkoxy, or C₁-C₈alkoxycarbonyl-, or two R⁵ on adjacent carbon atoms together form a —CH═CH—CH═CH— bridge, a —CH₂—CH₂—CH₂— bridge, a —CH(OH)—CH₂—CH₂— bridge, a —C(O)—CH₂—CH₂— bridge or a —N═CH—CH═CH— bridge;
R^{5a} and R^{5b} are, independently of each other, hydrogen, cyano, halogen, hydroxyl, C₁-C₈alkyl-, C₁-C₈alkyl- substituted by one to five R^{1a}, C₁-C₈alkylthio-, C₁-C₈haloalkylthio-, C₁-C₈alkylsulfinyl-, C₁-C₈haloalkylsulfinyl-, C₁-C₈alkylsulfonyl-, C₁-C₈haloalkylsulfonyl-, arylthio- or arylthio- wherein the aryl moiety is substituted by one to five R⁷, arylsulfinyl- or arylsulfinyl- wherein the aryl moiety is substituted by one to five R⁷, arylsulfonyl- or arylsulfonyl- wherein the aryl moiety is substituted by one to five R⁷, heterocyclylthio- or heterocyclylthio- wherein the heterocyclyl moiety is substituted by one to five R⁷, heterocyclylsulfinyl- or heterocyclylsulfinyl- wherein the heterocyclyl moiety is substituted by one to five R¹, or heterocyclylsulfonyl- or heterocyclylsulfonyl- wherein the heterocyclyl moiety is substituted by one to five R⁷, C₂-C₈alkenyl, C₂-C₈alkynyl, C₂-C₈haloalkenyl, C₁-C₈alkoxy, or C₁-C₈haloalkoxy, provided that at least one of R^{5a} and R^{5b} is not hydrogen;
R⁶ is hydrogen or C₁-C₈alkyl;
R^{6a} is independently halogen, cyano, nitro, amino, hydroxy, oxo, C₁-C₅alkylamino, hydroxyimino, C₁-C₈alkyloxyimino, di-C₁-C₈alkylamino, C₁-C₈alkoxy, acetyloxy, formyloxy, C₁-C₈haloalkoxy, C₁-C₄alkylthio or (C₁-C₄alkyl)₀₋₃silyl;
R⁷ is independently halogen, cyano, nitro, C₁-C₈alkyl, C₁-C₈haloalkyl, C₁-C₈alkoxy, C₁-C₈haloalkoxy; or an agrochemically acceptable salt, tautomer and N-oxide thereof.

Compounds of formula (I) which have at least one basic centre can form, for example, acid addition salts, for example with strong inorganic acids such as mineral acids, for example perchloric acid, sulfuric acid, nitric acid, nitrous acid, a phosphorus acid or a hydrohalic acid, with strong organic carboxylic acids, such as C₁-C₄alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄alkane- or arylsulfonic acids which are unsubstituted or substituted, for example by halogen, for example methane- or p-toluenesulfonic acid. Compounds of formula (I) which have at least one acidic group can form, for example, salts with bases, for example mineral salts such as alkali metal or alkaline earth metal salts, for example sodium, potassium or magnesium salts, or salts with ammonia or an organic amine, such as morpholine, piperidine, pyrrolidine, a mono-, di- or tri-lower-alkylamine, for example ethyl-, diethyl-, triethyl- or dimethylpropylamine, or a mono-, di- or trihydroxy-lower-alkylamine, for example mono-, di- or triethanolamine.

The alkyl groups occurring in the definitions of the substituents can be straight-chain or branched and are, for example, methyl, ethyl, n-propyl, isopropyl, n-butyl, sec-butyl, iso-butyl, tert-butyl, pentyl, hexyl, nonyl, decyl and their branched isomers. Alkylsulfanyl, alkylsulfinyl, alkylsulfonyl, alkylcarbonyl, alkoxy, alkenyl and alkynyl radicals are derived from the alkyl radicals mentioned. The alkenyl and alkynyl groups can be mono- or polyunsaturated.

Halogen is generally fluorine, chlorine, bromine or iodine. This also applies, correspondingly, to halogen in combination with other meanings, such as haloalkyl or halophenyl.

Haloalkyl groups preferably have a chain length of from 1 to 6 carbon atoms. Haloalkyl is, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl and 2,2,2-trichloroethyl.

Alkoxy is, for example, methoxy, ethoxy, propoxy, i-propoxy, n-butoxy, isobutoxy, sec-butoxy and tert-butoxy and also the isomeric pentyloxy and hexyloxy radicals.

Alkoxyalkyl groups preferably have a chain length of 1 to 6 carbon atoms. Alkoxyalkyl is, for example, methoxymethyl, methoxyethyl, ethoxymethyl, ethoxyethyl, n-propoxymethyl, n-propoxyethyl, isopropoxymethyl or isopropoxyethyl.

Alkoxycarbonyl is for example methoxycarbonyl (which is Cialkoxycarbonyl), ethoxycarbonyl, propoxycarbonyl, isopropoxycarbonyl, n-butoxycarbonyl, tert-butoxycarbonyl, n-pentoxycarbonyl or hexyloxycarbonyl.

The cycloalkyl groups preferably have from 3 to 6 ring carbon atoms, for example cyclopropyl, cyclobutyl, cyclopentyl and cyclohexyl.

As used herein, the term “C₂-C₈alkynyl” refers to a straight or branched hydrocarbon chain radical group consisting solely of carbon and hydrogen atoms, containing at least one triple bond, having from two to eight carbon atoms, and which is attached to the rest of the molecule by a single bond. Examples of C₂-C₈ alkynyl include, but are not limited to, ethynyl, prop-1-ynyl, but-1-ynyl and but-2-ynyl.

As used herein, the term “C₂-C₈alkenyl” refers to a straight or branched hydrocarbon chain radical group consisting solely of carbon and hydrogen atoms, containing at least one double bond, having from two to eight carbon atoms, and which is attached to the rest of the molecule by a single bond. Examples of C2-C₈alkenyl include, but are not limited to, prop-1-enyl, but-1-enyl and but-2-enyl.

As used herein, alkylcarbonyloxyalkyl refers to a group —ROC(O)R, wherein each R is, independently, C₁-C₈ alkyl.

As used herein, alkoxycarbonylsulfanyl refers to a group —SC(O)OR, wherein R is C₁-C₈ alkyl.

As used herein, alkylaminocarbonyloxyalkyl refers to a group —ROC(O)NHR, wherein each R is, independently, C₁-C₈ alkyl.

As used herein, dialkylaminocarbonyloxyalkyl refers to a group —ROC(O)NRR, wherein each R is, independently, C₁-C₈ alkyl.

As used herein, alkylaminocarbonylalkyl refers to a group —RC(O)NHR, wherein each R is, independently, C₁-C₈ alkyl.

As used herein, dialkylaminocarbonylalkyl refers to a group —RC(O)NRR, wherein each R is, independently, C₁-C₈ alkyl.

As used herein, alkoxycarbonylalkylaminoalkyl refers to a group —RNH(R)C(O)OR, wherein each R is, independently, C₁-C₈ alkyl.

Heteroaryl groups preferably are the 5-6 membered heteroaryls or the 5-6 membered heteroaryls substituted by one to three R⁷ where the heteroaryl groups contain 1 to 3 hetero atoms selected from the group consisting of nitrogen, oxygen and sulfur, it not being possible for each ring system to contain more than 2 oxygen atoms and more than 2 sulfur atoms. Examples of monocyclic groups include pyridyl, pyridazinyl, pyrimidinyl, pyrazinyl, pyrrolyl, pyrazolyl, imidazolyl, triazolyl (e.g. 1.2.4 triazolyl), furanyl, thiophenyl, oxazolyl, isoxazolyl, oxadiazolyl, thiazolyl, isothiazolyl, tetrazolyl and thiadiazolyl.

The compounds of formula (I) according to the invention also include hydrates which may be formed during the salt formation.

Preferred values of A¹, A², A³, A⁴, B¹—B²—B³—B⁴, R¹, R², R³, R⁴, R⁵, R^{6a}, R^{6b}, R⁷, R⁸, R⁹ and R¹⁰ in relation to each compound of the present invention, including the intermediate compounds, are, in any combination (including combinations of preferred values with the original values) as set out below.

Preferably no more than two of A¹, A², A³ and A⁴ are nitrogen.

Preferably A¹ is C—H or C—R⁵; more preferably A¹ is C—R⁵.

Preferably A² is C—H or C—R⁵; more preferably A² is C—H.

Preferably A³ is C—H or N; more preferably A³ is C—H.

Preferably A⁴ is C—H or N; more preferably A⁴ is C—H.

Preferably A¹ is C—R⁵; A² is C—H; A³ is C—H; and A⁴ is C—H.

Preferably each of A², A³ and A⁴ are C—H and A¹ is C—R⁵.

Preferably B¹—B²—B³—B⁴ is —CH═C—CH₂—O—.

Preferably B¹—B²—B³—B⁴—CH₂—C═CH—O—.

Preferably B¹—B²—B³—B⁴ is —CH₂—N—CH₂—CH₂—.

Preferably B¹—B²—B³—B⁴ is —CH₂—C═N—S—.

Preferably B¹—B²—B³—B⁴ is —CH₂—C═CH₂—S—.

Preferably B¹—B²—B³—B⁴ is —CH₂—C═N—CH₂—.

Preferably B¹—B²—B³—B⁴ is —C(R^{5a}R^{5b})—C═N—O—.

Preferably B¹—B²—B³—B⁴ is —CH(OH)—N—CH₂—CH₂—.

Preferably B¹—B²—B³—B⁴ is —C(O)—N—CH₂—CH₂—. Preferably R¹ is hydrogen, formyl, C₁-C₈alkyl, C₁-C₈alkylcarbonyl- or C₁-C₈alkoxycarbonyl-; more preferably hydrogen, methyl, ethyl, methylcarbonyl-, methoxymethyl, ethoxymethyl or methoxycarbonyl; most preferably hydrogen, methoxymethyl, ethoxymethyl, methyl or methoxycarbonyl; especially hydrogen or methoxymethyl; more especially hydrogen.

In a further embodiment, preferably, R¹ is C₁-C₈alkyl, C₁-C₈alkylcarbonyl-, C₃-C₆cycloalkylcarbonyl, C₁-C₈alkoxy, C₁-C₈alkoxy-C₁-C₈alkyl, C₁-C₈alkoxycarbonyl, C₁-C₈alkylcarbonyloxyC₁-C₈alkyl, C₁-C₈alkoxycarbonylsulfanyl, C₁-C₈alkylaminocarbonyloxyC₁-C₈alkyl, C₁-C₈dialkylaminocarbonyloxyC₁-C₈alkyl, C₁-C₈alkylaminocarbonylC₁-C₈alkyl, C₁-C₈dialkylaminocarbonylC₁-C₈alkyl, or C₁-C₈alkoxycarbonylC₁-C₈alkylaminoC₁-C₈alkyl, wherein each alkyl or alkoxy group may be optionally substituted with from one to three halogen atoms or with a cyano group; preferably, R¹ is C₁-C₈alkyl, C₁-C₈alkoxy-C₁-C₈alkyl, C₁-C₈alkoxycarbonyl, C₁-C₈alkylcarbonyloxyC₁-C₈alkyl, wherein each alkyl or alkoxy group may be optionally substituted with from one to three halogen atoms or with a cyano group, more preferably, R¹ is C₁-C₈cyanoalkyl, C₁-C₈alkoxy-C₁-C₈alkyl, C₁-C₈alkoxycarbonyl or C₁-C₈alkylcarbonyloxyC₁-C₈alkyl; most preferably, R¹ is methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl.

Preferably, R² is C₁-C₄alkyl or C₁-C₄haloalkyl, halogen, amino, C₁-C₄alkoxy, C₂-C₄alkenyl, C₂-C₄alkynyl, C₁-C₄haloalkoxy, C₁-C₄alkylamino, C₁-C₄alkylamino wherein the alkyl group is substituted by R^{6a}, C₁-C₄alkenylamino, —NHC(O)NR^{a}R^{b}, C₁-C₄dialkylamino, C₁-C₄dialkylamino wherein one or both of the alkyl groups are independently substituted by R^{6a}, C₁-C₄dialkenylamino, C₁-C₄haloalkylamino, C₁-C₄dihaloalkylamino, C₃-C₆cycloalkyl, C₁-C₄alkyl substituted by R^{6a}, C₂-C₄alkenyl substituted by R^{6B}, C₂-C₄alkynyl substituted by R^{6b}, C₃-C₆cycloalkyl substituted by R^{6b} or —R^{c}CO(O)R^{d}, wherein R^{a}, R^{b}, R^{c} and R^{d} are, independently, C₁-C₆ alkyl; more preferably R² is C₁-C₄alkyl or C₁-C₄haloalkyl, halogen, amino, C₂-C₄alkenyl, C₁-C₄haloalkoxy, C₁-C₄alkylamino, C₁-C₄dialkylamino, C₁-C₄haloalkylamino, C₁-C₄dihaloalkylamino, C₃-C₆cycloalkyl, C₁-C₄alkyl substituted by R^{6a}, or C₃-C₆cycloalkyl substituted by R^{6b}; especially R² is methyl, ethyl, propyl, isopropyl, CH₂F, CH₂Cl, CH₂Br, CHF₂, CF₃, CH₂CH₂CF₃, CH₂CF₃, halogen, amino, cyclopropyl, vinyl, dimethylamino, bis-2,2,2-trifluoroethylamino, bis-2,2-difluoroethylamino, methylamino, 2,2,2-trifluoroethylamino, 2,2-difluoroethylamino, amino, acetonitrile, 1-methoxymethylcyclopropan-1-yl or 2,2,2-trifluoroethoxy; more especially R² is methyl, ethyl, CH₂F, CH₂Cl, CH₂Br, CHF₂, fluorine, cyclopropyl, 1-cyanocyclopropyl, 1-methylcyclopropyl, cyclobutyl, dimethylamino, methylamino, ethylamine, isopropylamino, propylamino, isopropylmethylamino, cyclobutylmethylamino, cyclopropylmethylamino, bis-allylamino, bis-cyclopropylmethylamino, 2-methylpropanenitrile, bis-2,2,2-trifluoroethylamino, bis-2,2-difluoroethylamino methylamino, 2,2,2-trifluoroethylamino, 2,2-difluoroethylamino, 2,2,2-trifluoroethoxy, amino, methyl propanoate, methyl 2,2-dimethylpropanoate or acetonitrile; even more especially R² is methyl, ethyl, CH₂F, dimethylamino, CHF₂, fluorine, cyclopropyl or 1-cyanocyclopropyl,

Preferably R³ is C₁-C₄haloalkyl; more preferably chlorodifluoromethyl or trifluoromethyl; most preferably trifluoromethyl.

R⁴ is preferably phenyl or phenyl substituted by one to three R^{6b}; more preferably R⁴ is phenyl substituted by one to three substituents selected from F, Cl, Br or CF₃; even more preferably R⁴ is phenyl substituted by one to three R^{6b}; most preferably R⁴ is 3,5-bis-(trifluoromethyl)-phenyl, 3-chloro-5-trifluoromethyl-phenyl, 3-bromo-5-trifluoromethyl-phenyl, 3,5-dibromo-phenyl, 3,5-dichloro-phenyl, 3,4-dichloro-phenyl, 3-trifluoromethyl-phenyl, 4-bromo-3,5-dichlorophenyl, 4-fluoro-3,5-dichlorophenyl or 3,4,5-trichloro-phenyl; more especially R⁴ is 3-chloro-5-trifluoromethyl-phenyl, 3,5-dichloro-phenyl, 3,5-bis-(trifluoromethyl)-phenyl, 3,5-dichloro-4-fluoro-phenyl or 3,4,5-trichloro-phenyl.

Preferably R⁵ is independently halogen, cyano, nitro, C₁-C₈alkyl, C₃-C₈cycloalkyl, C₁-C₈haloalkyl, or C₂-C₈alkenyl, or two R⁵ on adjacent carbon atoms together form a —CH═CH—CH═CH— bridge or a —CH₂—CH₂—CH₂- bridge; more preferably R⁵ is independently halogen, cyano, C₁-C₈alkyl, C₃-Ccycloalkyl, C₁-C₈haloalkyl or C₂-C₈alkenyl; even more preferably R⁵ is halogen, cyano, C₁-C₈alkyl, C₂-C₈ alkenyl, C₃-C₈cycloalkyl or C₁-C₈haloalkyl.

In a further embodiment R⁵ is independently fluoro, chloro, bromo, trifluoromethyl, methyl or two R⁵ on adjacent carbon atoms together form a —CH═CH—CH═CH— bridge or a —CH₂—CH₂—CH₂— bridge; more preferably R⁵ is bromo, chloro, fluoro, cyclopropyl, trifluoromethyl, vinyl, ethyl or methyl; most preferably chloro, bromo, trifluoromethyl, ethyl or methyl.

Preferably R^{1a} is halogen, hydroxyl, C₁-C₈alkylthio-, C₁-C₈haloalkylthio-, C₁-C₈alkylsulfinyl-, C₁-C₈haloalkylsulfinyl-, C₁-C₈alkylsulfonyl-, C₁-C₈haloalkylsulfonyl-, C₁-C₈alkyl, C₂-C₈alkenyl, C₂-C₈alkynyl, C₁-C₈haloalkyl, C₂-C₈haloalkenyl, most preferably halogen, C₁-C₈alkylthio- or C₁-C₈alkyl, even most preferably halogen.

Preferably R^{5b} is halogen or hydrogen, most preferably hydrogen.

Preferably R^{6a} independently is cyano, halogen, C₁-C₄alkoxy, C₁-C₄haloalkoxy or C₁-C₄alkylthio; more preferably fluoro, cyano, methoxy, difluoromethoxy, trifluoromethoxy or methylthio.

Preferably R^{6b} independently is halogen, cyano, C₁-C₄alkyl, C₁-C₄haloalkyl, C₁-C₄alkoxy, or C₁-C₄haloalkoxy; more preferably bromo, chloro, fluoro, cyano, methyl, ethyl, trifluoromethyl, methoxy, difluoromethoxy, trifluoromethoxy; more preferably bromo, chloro, fluoro, cyano, methyl, ethyl, trifluoromethyl, methoxy, or trifluoromethoxy; especially bromo, chloro, fluoro, cyano, methyl, ethyl, trifluoromethyl, methoxy, or trifluoromethoxy; more especially fluoro, chloro, bromo, cyano or trifluoromethyl.

Preferably R⁷ are independently from each other halogen, cyano, C₁-C₄alkyl, C₁-C₄haloalkyl, C₁-C₄alkoxy or C₁-C₄haloalkoxy; more preferably, methyl, fluoro, chloro, bromo, trifluoromethyl, trifluoromethoxy, cyano or methoxy.

In an embodiment E1 of formula (I), independent of other embodiments, A¹ is CR⁵ and A², A³ and A⁴ are each CH, wherein R⁵ is as defined under formula (I) above.

In an embodiment E2 of formula (I), independent of other embodiments, R¹ is hydrogen, formyl, C₁-C₈alkyl, C₁-C₈alkylcarbonyl-, C₁-C₈alkoxyalkyl- or C₁-C₈alkoxycarbonyl.

In an embodiment E2a of formula (I), independent of other embodiments, R¹ is C₁-C₈alkyl, C₁-C₈alkylcarbonyl-, C₃-C₆cycloalkylcarbonyl, C₁-C₈alkoxy, C₁-C₈alkoxy-C₁-C₈alkyl, C₁-C₈alkoxycarbonyl, C₁-C₈alkylcarbonyloxyC₁-C₈alkyl, C₁-C₈alkoxycarbonylsulfanyl, C₁-C₈alkylaminocarbonyloxyC-C₈alkyl, C₁-C₈dialkylaminocarbonyloxyC₁-C₈alkyl, C₁-C₈alkylaminocarbonylC₁-C₈alkyl, C₁-C₈dialkylaminocarbonylC₁-C₈alkyl, or C₁-C₈alkoxycarbonylC₁-C₈alkylaminoC₁-C₈alkyl, wherein each alkyl or alkoxy group may be optionally substituted with from one to three halogen atoms or with a cyano group.

In an embodiment E3 of formula (I), independent of other embodiments, R² is selected from C₁-C₄alkyl, C₁-C₄haloalkyl, halogen, amino, C₂-C₄alkenyl, C₁-C₄alkylamino, C₁-C₄dialkylamino, C₁-C₄haloalkylamino, C₁-C₄dihaloalkylamino, C₃-C₆cycloalkyl, C₁-C₄alkyl substituted by R^{6a} or C₃-C₆cycloalkyl substituted by R^{6b}.

In an embodiment E4 of formula (I), independent of other embodiments, R³ is C₁-C₄haloalkyl.

In an embodiment E5 of formula (I), independent of other embodiments, R⁴ is phenyl or phenyl substituted by one to three R^{6b}, wherein R^{6b} is as defined under formula (I) above.

Embodiment E6 of formula (I) provides compounds of formula (I) wherein A¹ is CR⁵ and A², A³ and A⁴ are each CH; R¹ is hydrogen, methyl, ethyl, methoxymethyl, ethoxymethyl, methylcarbonyl-, or methoxycarbonyl; R² is selected from C₁-C₄alkyl, C₁-C₄haloalkyl, halogen, amino, C₂-C₄alkenyl, C₁-C₄alkylamino, C₁-C₄dialkylamino, C₁-C₄haloalkylamino, C₁-C₄dihaloalkylamino, C₃-C₆cycloalkyl, C₁-C₄alkyl substituted by R^{6a} or C₃-C₆cycloalkyl substituted by R^{6b}, R³ is C₁-C₄haloalkyl; R⁴ is phenyl or phenyl substituted by one to three R^{6b}; wherein R⁵ is halogen or C₁-C₈alkyl, C₃-C₈cycloalkyl, C₁-C₈haloalkyl or C₂-C₈alkenyl; R^{6a} is independently cyano, C₁-C₄alkoxy or C₁-C₄haloalkoxy; and R^{6b} is independently halogen, cyano, C₁-C₄alkyl, or C₁-C₄haloalkyl, C₁-C₄alkylthio, C₁-C₄alkoxy or C₁-C₄haloalkoxy.

Embodiment E7 of formula (I) provides compounds of formula (I) wherein A¹ is CR⁵ and A², A³ and A⁴ are each CH; R¹ is hydrogen, methyl, ethyl, methoxymethyl, ethoxymethyl, methylcarbonyl-, or methoxycarbonyl; R² is selected from C₁-C₄alkyl, C₁-C₄haloalkyl, halogen, amino, C₂-C₄alkenyl, C₁-C₄alkylamino, C₁-C₄dialkylamino, C₁-C₄haloalkylamino, C₁-C₄dihaloalkylamino, C₃-C₆cycloalkyl, C₁-C₄alkyl substituted by R^{6a}, or C₃-C₆cycloalkyl substituted by R^{6b}; R³ is C₁-C₄haloalkyl; R⁴ is phenyl or phenyl substituted by one to three R^{6b}; wherein R⁵ is halogen, cyano, C₁-C₄alkyl, C₃-C₆cycloalkyl, C₁-C₄haloalkyl, C₂-C₄alkenyl; R^{6a} is independently cyano, methoxy, difluoromethoxy or trifluoromethoxy; and R^{6b} is independently bromo, chloro, fluoro, cyano, methyl, ethyl, trifluoromethyl, difluoromethoxy or trifluoromethoxy.

Embodiment E8 of formula (I) provides compounds of formula (I) wherein A¹ is CR⁵ and A², A³ and A⁴ are each CH; R¹ is hydrogen; R² is selected from C₁-C₄alkyl, C₁-C₄haloalkyl, halogen, amino, C₂-C₄alkenyl, C₁-C₄alkylamino, C₁-C₄dialkylamino, C₁-C₄haloalkylamino, C₁-C₄dihaloalkylamino, C₃-C₆cycloalkyl, C₁-C₄alkyl substituted by R^{6a}, or C₃-C₆cycloalkyl substituted by R^{6b}; R³ is chlorodifluoromethyl or trifluoro-methyl; R⁴ is 3,5-bis-(trifluoromethyl)-phenyl, 3-chloro-5-trifluoromethyl-phenyl, 3-bromo-5-trifluoromethyl-phenyl, 3,5-dibromo-phenyl, 3,5-dichloro-phenyl, 3,4-dichloro-phenyl, 3-trifluoromethyl-phenyl, 4-bromo-3,5-dichlorophenyl, 3,5-dichloro-4-fluoro-phenyl or 3,4,5-trichloro-phenyl; and wherein R⁵ is bromo, chloro, fluoro, cyano, methyl, ethyl, trifluoromethyl, cyclopropyl, or vinyl; R^{6a} is independently cyano, methoxy, difluoromethoxy or trifluoromethoxy; and R^{6b} is independently bromo, chloro, fluoro, cyano, methyl, ethyl, trifluoromethyl, difluoromethoxy or trifluoromethoxy.

Embodiment E9 of formula (I) provides compounds of formula (I) wherein A¹ is CR⁵ and A², A³ and A⁴ are each CH; R¹ is hydrogen; R² is selected from C₁-C₄alkyl, C₁-C₄haloalkyl, halogen, amino, C₂-C₄alkenyl, C₁-C₄alkylamino, C₁-C₄dialkylamino, C₁-C₄haloalkylamino, C₁-C₄dihaloalkylamino, C₃-C₆cycloalkyl, C₁-C₄alkyl substituted by R^{6a}, or C₃-C₆cycloalkyl substituted by R^{6b}; R³ is chlorodifluoromethyl or trifluoro-methyl; R⁴ is 3,5-bis-(trifluoromethyl)-phenyl, 3-chloro-5-trifluoromethyl-phenyl, 3-bromo-5-trifluoromethyl-phenyl, 3,5-dibromo-phenyl, 3,5-dichloro-phenyl, 3,4-dichloro-phenyl, 3-trifluoromethyl-phenyl, 4-bromo-3,5-dichlorophenyl, 3,5-dichloro-4-fluoro-phenyl or 3,4,5-trichloro-phenyl; wherein R⁵ is bromo, chloro, fluoro, cyano, methyl, ethyl, trifluoromethyl, cyclopropyl, or vinyl; R^{6a} is independently fluoro, cyano, methoxy, difluoromethoxy or trifluoromethoxy; and R^{6b} is independently bromo, chloro, fluoro, cyano, methyl, ethyl, trifluoromethyl, difluoromethoxy or trifluoromethoxy.

Embodiment E10 of formula (I) provides compounds of formula (I) wherein A¹ is CR⁵ and A², A³ and A⁴ are each CH; R¹ is hydrogen; R² is selected from methyl, ethyl, CH₂F, CH₂Cl, CH₂Br, CHF₂, fluorine, cyclopropyl, 1-cyanocyclopropyl, 1-methylcyclopropyl, cyclobutyl, dimethylamino, methylamino, ethylamine, isopropylamino, propylamino, isopropylmethylamino, cyclobutylmethylamino, cyclopropylmethylamino, bis-allylamino, bis-cyclopropylmethylamino, 2-methylpropanenitrile, bis-2,2,2-trifluoroethylamino, bis-2,2-difluoroethylamino methylamino, 2,2,2-trifluoroethylamino, 2,2-difluoroethylamino, 2,2,2-trifluoroethoxy, amino, methyl propanoate, methyl 2,2-dimethylpropanoate or acetonitrile; R⁴ is 3,5-bis-(trifluoromethyl)-phenyl, 3-chloro-5-trifluoromethyl-phenyl, 3-bromo-5-trifluoromethyl-phenyl, 3,5-dibromo-phenyl, 3,5-dichloro-phenyl, 3,5-dichloro-4-fluoro-phenyl, 3,4-dichloro-phenyl, 3-trifluoromethyl-phenyl, 4-bromo-3,5-dichlorophenyl, 4-fluoro-3,5-dichlorophenyl or 3,4,5-trichloro-phenyl; wherein R⁵ is bromo, chloro, fluoro, cyano, methyl, ethyl, trifluoromethyl, cyclopropyl, or vinyl.

Embodiment E11 of formula (I) provides compounds of formula (I) wherein A², A³ and A⁴ are each CH; R¹ is hydrogen; R³ is chlorodifluoromethyl or trifluoromethyl; A¹ is CR⁵, wherein R⁵ is chloro, bromo, trifluoromethyl, or methyl; R² is selected from methyl, ethyl, CH₂F, dimethylamino, CHF₂, fluorine, cyclopropyl or 1-cyanocyclopropyl; and R⁴ is 3-chloro-5-trifluoromethyl-phenyl, 3-bromo-5-trifluoromethyl-phenyl, 3-trifluoromethyl-phenyl, 3,5-dibromo-phenyl, 3,5-dichloro-phenyl, 3,5-bis-(trifluoromethyl)-phenyl, 4-bromo-3,5-dichlorophenyl, 3,4,5-trichloro-phenyl, 3,5-dichloro-4-fluoro-phenyl, pyridyl, or 3-5-substituted pyridyl wherein the substituents are selected from bromo, chloro, fluoro and trifluoromethyl.

Embodiment E12 of formula (I) provides compounds of formula (I) wherein A², A³ and A⁴ are each CH; R¹ is hydrogen; R³ is trifluoromethyl; A¹ is CR⁵, wherein R⁵ is chloro or methyl; R² is selected from methyl, ethyl, CH₂F, dimethylamino, CHF₂, fluorine, cyclopropyl or 1-cyanocyclopropyl; and R⁴ is 3,5-bis-(trifluoromethyl)-phenyl, 3-chloro-5-trifluoromethyl-phenyl, 3-bromo-5-trifluoromethyl-phenyl, 3,5-dibromo-phenyl, 3,5-dichloro-phenyl, 3,4-dichloro-phenyl, 3-trifluoromethyl-phenyl, 4-bromo-3,5-dichlorophenyl, 4-fluoro-3,5-dichlorophenyl or 3,4,5-trichloro-phenyl, 3,5-dichloro-4-fluoro-phenyl; especially R⁴ is 3,5-dibromo-phenyl, 3,5-dichloro-phenyl, 3,5-bis-(trifluoromethyl)-phenyl, 3-chloro-5-trifluoromethyl-phenyl, 3-bromo-5-trifluoromethyl-phenyl, 4-bromo-3,5-dichlorophenyl, 3,4,5-trichloro-phenyl, 3,5-dichloro-4-fluoro-phenyl, pyridyl, or 3-5-substituted pyridyl wherein the substituents are selected from the group consisting of bromo, chloro, fluoro and trifluoromethyl.

Embodiment E13 of formula (I) provides compounds of formula (I) wherein A², A³ and A⁴ are each CH; R¹ is hydrogen; R³ is chlorodifluoromethyl or trifluoromethyl; A¹ is CR⁵, wherein R⁵ is chloro, bromo, trifluoromethyl, fluoro, or methyl; R² is selected from methyl, ethyl, CH₂F, dimethylamino, CHF₂, fluorine, cyclopropyl or 1-cyanocyclopropyl; and R⁴ is phenyl, pyridyl, mono-, di- or tri-substituted phenyl where the substituents are independently selected from the group consisting of chloro, bromo, fluoro and trifluoromethyl, or mono- or di-substituted pyridyl where the substituents are independently selected from chloro, bromo, fluoro and trifluoromethyl.

Embodiment E14 of formula (I) provides compounds of formula (I) wherein A², A³ and A⁴ are each CH; R¹ is hydrogen; R³ is trifluoromethyl; A¹ is CR⁵, wherein R⁵ is chloro or methyl; R² is independently selected from methyl, ethyl, CH₂F, dimethylamino, CHF₂, fluorine, cyclopropyl or 1-cyanocyclopropyl; and R⁴ is 3,5-dichloro-4-fluoro-phenyl, 3,5-dichloro-phenyl, 3,4,5-trichloro-phenyl, 3-bromo-5-trifluoromethyl-phenyl, 3-trifluoromethyl-phenyl or 3-chloro-5-trifluoromethyl-phenyl.

Embodiment E15 of formula (I) provides compounds of formula (I) wherein A², A³ and A⁴ are each CH; R¹ is hydrogen; R³ is trifluoromethyl; A¹ is CR⁵, wherein R⁵ is chloro; R² is selected from methyl, ethyl, CH₂F, dimethylamino, CHF₂, fluorine, cyclopropyl or 1-cyanocyclopropyl; and R⁴ is 3,5-bis-(trifluoromethyl)-phenyl, 3-chloro-5-trifluoromethyl-phenyl, 3-bromo-5-trifluoromethyl-phenyl, 3,5-dibromo-phenyl, 3,5-dichloro-phenyl, 3,4-dichloro-phenyl, 3-trifluoromethyl-phenyl, 4-bromo-3,5-dichlorophenyl, 4-fluoro-3,5-dichlorophenyl or 3,4,5-trichloro-phenyl, 3,5-dichloro-4-fluoro-phenyl; especially R⁴ is 3,5-dibromo-phenyl, 3,5-dichloro-phenyl, 3,5-bis-(trifluoromethyl)-phenyl, 3-chloro-5-trifluoromethyl-phenyl, 3-bromo-5-trifluoromethyl-phenyl, 4-bromo-3,5-dichlorophenyl, 3,4,5-trichloro-phenyl, 3,5-dichloro-4-fluoro-phenyl, pyridyl, or 3-5-substituted pyridyl wherein the substituents are selected from the group consisting of bromo, chloro, fluoro and trifluoromethyl.

Embodiment E16 of formula (I) provides compounds of formula (I) wherein A², A³ and A⁴ are each CH; R¹ is hydrogen; R³ is chlorodifluoromethyl or trifluoromethyl; A¹ is CR⁵, wherein R⁵ is chloro; R² is selected from methyl, ethyl, CH₂F, dimethylamino, CHF₂ or fluorine, and R⁴ is phenyl, pyridyl, mono-, di- or tri-substituted phenyl where the substituents are independently selected from the group consisting of chloro, bromo, fluoro and trifluoromethyl, or mono- or di-substituted pyridyl where the substituents are independently selected from chloro, bromo, fluoro and trifluoromethyl.

Embodiment E17 of formula (I) provides compounds of formula (I) wherein A², A³ and A⁴ are each CH; R¹ is hydrogen; R³ is trifluoromethyl; A¹ is CR⁵, wherein R⁵ is chloro; R² is independently selected from methyl, ethyl, CH₂F, dimethylamino, CHF₂ or fluorine; and R⁴ is 3,5-dichloro-4-fluoro-phenyl, 3,5-dichloro-phenyl, 3,4,5-trichloro-phenyl, 3-bromo-5-trifluoromethyl-phenyl, 3-trifluoromethyl-phenyl or 3-chloro-5-trifluoromethyl-phenyl.

Embodiment E18 of formula (I) provides compounds of formula (I) wherein A², A³ and A⁴ are each CH; R¹ is hydrogen; R³ is trifluoromethyl; A¹ is CR⁵, wherein R⁵ is bromo; R² is selected from methyl, ethyl, CH₂F, dimethylamino, CHF₂, fluorine, cyclopropyl or 1-cyanocyclopropyl; and R⁴ is 3,5-bis-(trifluoromethyl)-phenyl, 3-chloro-5-trifluoromethyl-phenyl, 3-bromo-5-trifluoromethyl-phenyl, 3,5-dibromo-phenyl, 3,5-dichloro-phenyl, 3,4-dichloro-phenyl, 3-trifluoromethyl-phenyl, 4-bromo-3,5-dichlorophenyl, 4-fluoro-3,5-dichlorophenyl or 3,4,5-trichloro-phenyl, 3,5-dichloro-4-fluoro-phenyl; especially R⁴ is 3,5-dibromo-phenyl, 3,5-dichloro-phenyl, 3,5-bis-(trifluoromethyl)-phenyl, 3-chloro-5-trifluoromethyl-phenyl, 3-bromo-5-trifluoromethyl-phenyl, 4-bromo-3,5-dichlorophenyl, 3,4,5-trichloro-phenyl, 3,5-dichloro-4-fluoro-phenyl, pyridyl, or 3-5-substituted pyridyl wherein the substituents are selected from the group consisting of bromo, chloro, fluoro and trifluoromethyl.

Embodiment E19 of formula (I) provides compounds of formula (I) wherein A², A³ and A⁴ are each CH; R¹ is hydrogen; R³ is chlorodifluoromethyl or trifluoromethyl; A¹ is CR⁵, wherein R⁵ is bromo; R² is selected from methyl, ethyl, CH₂F, dimethylamino, CHF₂, fluorine, cyclopropyl or 1-cyanocyclopropyl, and R⁴ is phenyl, pyridyl, mono-, di- or tri-substituted phenyl where the substituents are independently selected from the group consisting of chloro, bromo, fluoro and trifluoromethyl, or mono- or di-substituted pyridyl where the substituents are independently selected from chloro, bromo, fluoro and trifluoromethyl.

Embodiment E20 of formula (I) provides compounds of formula (I) wherein A², A³ and A⁴ are each CH; R¹ is hydrogen; R³ is trifluoromethyl; A¹ is CR⁵, wherein R⁵ is bromo; R² is independently selected from methyl, ethyl, CH₂F, dimethylamino, CHF₂ or fluorine; and R⁴ is 3,5-dichloro-4-fluoro-phenyl, 3,5-dichloro-phenyl, 3,4,5-trichloro-phenyl, 3-bromo-5-trifluoromethyl-phenyl, 3-trifluoromethyl-phenyl or 3-chloro-5-trifluoromethyl-phenyl.

Embodiment E21 of formula (I) provides compounds of formula (I) wherein A², A³ and A⁴ are each CH; R¹ is hydrogen; R³ is trifluoromethyl; A¹ is CR⁵, wherein R⁵ is trifluoromethyl; R² is selected from methyl, ethyl, CH₂F, dimethylamino, CHF₂, fluorine, cyclopropyl or 1-cyanocyclopropyl; and R⁴ is 3,5-bis-(trifluoromethyl)-phenyl, 3-chloro-5-trifluoromethyl-phenyl, 3-bromo-5-trifluoromethyl-phenyl, 3,5-dibromo-phenyl, 3,5-dichloro-phenyl, 3,4-dichloro-phenyl, 3-trifluoromethyl-phenyl, 4-bromo-3,5-dichlorophenyl, 4-fluoro-3,5-dichlorophenyl or 3,4,5-trichloro-phenyl, 3,5-dichloro-4-fluoro-phenyl; especially R⁴ is 3,5-dibromo-phenyl, 3,5-dichloro-phenyl, 3,5-bis-(trifluoromethyl)-phenyl, 3-chloro-5-trifluoromethyl-phenyl, 3-bromo-5-trifluoromethyl-phenyl, 4-bromo-3,5-dichlorophenyl, 3,4,5-trichloro-phenyl, 3,5-dichloro-4-fluoro-phenyl, pyridyl, or 3-5-substituted pyridyl wherein the substituents are selected from the group consisting of bromo, chloro, fluoro and trifluoromethyl.

Embodiment E22 of formula (I) provides compounds of formula (I) wherein A², A³ and A⁴ are each CH; R¹ is hydrogen; R³ is chlorodifluoromethyl or trifluoromethyl; A¹ is CR⁵, wherein R⁵ is trifluoromethyl; R² is selected from methyl, ethyl, CH₂F, dimethylamino, CHF₂, fluorine, cyclopropyl or 1-cyanocyclopropyl, and R⁴ is phenyl, pyridyl, mono-, di- or tri-substituted phenyl where the substituents are independently selected from the group consisting of chloro, bromo, fluoro and trifluoromethyl, or mono- or di-substituted pyridyl where the substituents are independently selected from chloro, bromo, fluoro and trifluoromethyl.

Embodiment E23 of formula (I) provides compounds of formula (I) wherein A², A³ and A⁴ are each CH; R¹ is hydrogen; R³ is trifluoromethyl; A¹ is CR⁵, wherein R⁵ is trifluoromethyl; R² is independently selected from methyl, ethyl, CH₂F, dimethylamino, CHF₂ or fluorine; and R⁴ is 3,5-dichloro-4-fluoro-phenyl, 3,5-dichloro-phenyl, 3,4,5-trichloro-phenyl, 3-bromo-5-trifluoromethyl-phenyl, 3-trifluoromethyl-phenyl or 3-chloro-5-trifluoromethyl-phenyl.

Embodiment E24 of formula (I) provides compounds of formula (I) wherein A², A³ and A⁴ are each CH; R¹ is hydrogen; R³ is trifluoromethyl; A¹ is CR⁵, wherein R⁵ is methyl; R² is selected from methyl, ethyl, CH₂F, dimethylamino, CHF₂, fluorine, cyclopropyl or 1-cyanocyclopropyl; and R⁴ is 3,5-bis-(trifluoromethyl)-phenyl, 3-chloro-5-trifluoromethyl-phenyl, 3-bromo-5-trifluoromethyl-phenyl, 3,5-dibromo-phenyl, 3,5-dichloro-phenyl, 3,4-dichloro-phenyl, 3-trifluoromethyl-phenyl, 4-bromo-3,5-dichlorophenyl, 4-fluoro-3,5-dichlorophenyl or 3,4,5-trichloro-phenyl, 3,5-dichloro-4-fluoro-phenyl; especially R⁴ is 3,5-dibromo-phenyl, 3,5-dichloro-phenyl, 3,5-bis-(trifluoromethyl)-phenyl, 3-chloro-5-trifluoromethyl-phenyl, 3-bromo-5-trifluoromethyl-phenyl, 4-bromo-3,5-dichlorophenyl, 3,4,5-trichloro-phenyl, 3,5-dichloro-4-fluoro-phenyl, pyridyl, or 3-5-substituted pyridyl wherein the substituents are selected from the group consisting of bromo, chloro, fluoro and trifluoromethyl.

Embodiment E25 of formula (I) provides compounds of formula (I) wherein A², A³ and A⁴ are each CH; R¹ is hydrogen; R³ is chlorodifluoromethyl or trifluoromethyl; A¹ is CR⁵, wherein R⁵ is methyl; R² is selected from methyl, ethyl, CH₂F, dimethylamino, CHF₂, fluorine, cyclopropyl or 1-cyanocyclopropyl, and R⁴ is phenyl, pyridyl, mono-, di- or tri-substituted phenyl where the substituents are independently selected from the group consisting of chloro, bromo, fluoro and trifluoromethyl, or mono- or di-substituted pyridyl where the substituents are independently selected from chloro, bromo, fluoro and trifluoromethyl.

Embodiment E26 of formula (I) provides compounds of formula (I) wherein A², A³ and A⁴ are each CH; R¹ is hydrogen; R³ is trifluoromethyl; A¹ is CR⁵, wherein R⁵ is methyl; R² is independently selected from methyl, ethyl, CH₂F, dimethylamino, CHF₂ or fluorine; and R⁴ is 3,5-dichloro-4-fluoro-phenyl, 3,5-dichloro-phenyl, 3,4,5-trichloro-phenyl, 3-bromo-5-trifluoromethyl-phenyl, 3-trifluoromethyl-phenyl or 3-chloro-5-trifluoromethyl-phenyl.

Embodiment E27 of formula (I) provides compounds of formula (I) wherein A¹ is CR⁵ and A², A³ and A⁴ are each CH; R¹ is methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl; R² is selected C₁-C₄alkyl, C₁-C₄haloalkyl, halogen, amino, C₂-C₄alkenyl, C₁-C₄alkylamino, C₁-C₄dialkylamino, C₁-C₄haloalkylamino, C₁-C₄dihaloalkylamino, C₃-C₆cycloalkyl, C₁-C₄alkyl substituted by R^{6a}, or C₃-C₆cycloalkyl substituted by R^{6b}; R⁴ is phenyl or phenyl substituted by one to three R^{6b}; wherein R⁵ is halogen or C₁-C₈alkyl, C₃-Ccycloalkyl, C₁-C₈haloalkyl, or C₂-C₈alkenyl; R^{6a} is independently halogen, cyano, C₁-C₄alkoxy, or C₁-C₄haloalkoxy; and R^{6b} is independently halogen, cyano, C₁-C₄alkyl, or C₁-C₄haloalkyl, C₁-C₄alkylthio, C₁-C₄alkoxy, or C₁-C₄haloalkoxy.

Embodiment E28 of formula (I) provides compounds of formula (I) wherein A¹ is CR⁵ and A², A³ and A⁴ are each CH; R¹ is methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl; R² is selected C₁-C₄alkyl, C₁-C₄haloalkyl, halogen, amino, C₂-C₄alkenyl, C₁-C₄alkylamino, C₁-C₄dialkylamino, C₁-C₄haloalkylamino, C₁-C₄dihaloalkylamino, C₃-C₆cycloalkyl, C₁-C₄alkyl substituted by R^{6a}, or C₃-C₆cycloalkyl substituted by R^{6b}; R³ is C₁-C₄haloalkyl; R⁴ is phenyl or phenyl substituted by one to three R^{6b}; wherein R⁵ is halogen, cyano, C₁-C₄alkyl, C₃-C₆cycloalkyl, C₁-C₄haloalkyl, C₂-C₄alkenyl; R^{6a} is independently halogen, cyano, methoxy, difluoromethoxy or trifluoromethoxy; and R^{6b} is independently bromo, chloro, fluoro, cyano, methyl, ethyl, trifluoromethyl, methoxy, difluoromethoxy, trifluoromethoxy.

Embodiment E29 of formula (I) provides compounds of formula (I) wherein A¹ is CR⁵ and A², A³ and A⁴ are each CH; R¹ is methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl; R² is selected from C₁-C₄alkyl, C₁-C₄haloalkyl, halogen, amino, C₂-C₄alkenyl, C₁-C₄alkylamino, C₁-C₄dialkylamino, C₁-C₄haloalkylamino, C₁-C₄dihaloalkylamino, C₃-C₆cycloalkyl, C₁-C₄alkyl substituted by R^{6a}, or C₃-C₆cycloalkyl substituted by R^{6b}; R³ is chlorodifluoromethyl or trifluoromethyl; R⁴ is 3,5-bis-(trifluoromethyl)-phenyl, 3-chloro-5-trifluoromethyl-phenyl, 3-bromo-5-trifluoromethyl-phenyl, 3,5-dibromo-phenyl, 3,5-dichloro-phenyl, 3,4-dichloro-phenyl, 3-trifluoromethyl-phenyl, 4-bromo-3,5-dichlorophenyl, 3,5-dichloro-4-fluoro-phenyl or 3,4,5-trichloro-phenyl; and wherein R⁵ is bromo, chloro, fluoro, cyano, methyl, ethyl, trifluoromethyl, cyclopropyl, or vinyl; R^{6a} is independently halogen, cyano, methoxy, difluoromethoxy or trifluoromethoxy;

Embodiment E30 of formula (I) provides compounds of formula (I) wherein A¹ is CR⁵ and A², A³ and A⁴ are each CH; R¹ is methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl; R² is selected from C₁-C₄alkyl, C₁-C₄haloalkyl, halogen, amino, C₂-C₄alkenyl, C₁-C₄alkylamino, C₁-C₄dialkylamino, C₁-C₄haloalkylamino, C₁-C₄dihaloalkylamino, C₃-C₆cycloalkyl, C₁-C₄alkyl substituted by R^{6a}, or C₃-C₆cycloalkyl substituted by R^{6b}; R³ is chlorodifluoromethyl or trifluoromethyl; R⁴ is 3,5-bis-(trifluoromethyl)-phenyl, 3-chloro-5-trifluoromethyl-phenyl, 3-bromo-5-trifluoromethyl-phenyl, 3,5-dibromo-phenyl, 3,5-dichloro-phenyl, 3,4-dichloro-phenyl, 3-trifluoromethyl-phenyl, 4-bromo-3,5-dichlorophenyl, 3,5-dichloro-4-fluoro-phenyl or 3,4,5-trichloro-phenyl; wherein R⁵ is bromo, chloro, fluoro, cyano, methyl, ethyl, trifluoromethyl, cyclopropyl, or vinyl; R^{6a} is independently fluoro, cyano, methoxy, difluoromethoxy or trifluoromethoxy;

Embodiment E31 of formula (I) provides compounds of formula (I) wherein A¹ is CR⁵ and A², A³ and A⁴ are each CH; R¹ is methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl; R² is selected from methyl, ethyl, CH₂F, dimethylamino, CHF₂, fluorine, cyclopropyl or 1-cyanocyclopropyl; R⁴ is 3,5-bis-(trifluoromethyl)-phenyl, 3-chloro-5-trifluoromethyl-phenyl, 3-bromo-5-trifluoromethyl-phenyl, 3,5-dibromo-phenyl, 3,5-dichloro-phenyl, 3,5-dichloro-4-fluoro-phenyl, 3,4-dichloro-phenyl, 3-trifluoromethyl-phenyl, 4-bromo-3,5-dichlorophenyl, 4-fluoro-3,5-dichlorophenyl or 3,4,5-trichloro-phenyl; wherein R⁵ is bromo, chloro, fluoro, cyano, methyl, ethyl, trifluoromethyl, cyclopropyl, or vinyl;

Embodiment E32 of formula (I) provides compounds of formula (I) wherein A², A¹ and A⁴ are each CH; R¹ is methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl; R³ is chlorodifluoromethyl or trifluoromethyl; A¹ is CR⁵, wherein R⁵ is chloro, bromo, trifluoromethyl, or methyl; R² is selected from methyl, ethyl, CH₂F, dimethylamino, CHF₂, fluorine, cyclopropyl or 1-cyanocyclopropyl; and R⁴ is 3-chloro-5-trifluoromethyl-phenyl, 3-bromo-5-trifluoromethyl-phenyl, 3-trifluoromethyl-phenyl, 3,5-dibromo-phenyl, 3,5-dichloro-phenyl, 3,5-bis-(trifluoromethyl)-phenyl, 4-bromo-3,5-dichlorophenyl, 3,4,5-trichloro-phenyl, 3,5-dichloro-4-fluoro-phenyl, pyridyl, or 3-5-substituted pyridyl wherein the substituents are selected from bromo, chloro, fluoro and trifluoromethyl.

Embodiment E33 of formula (I) provides compounds of formula (I) wherein A², A³ and A⁴ are each CH; R¹ is methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl; R³ is trifluoromethyl; A¹ is CR⁵, wherein R⁵ is chloro or methyl; R² is selected from methyl, ethyl, CH₂F, dimethylamino, CHF₂, fluorine, cyclopropyl or 1-cyanocyclopropyl; and R⁴ is 3,5-bis-(trifluoromethyl)-phenyl, 3-chloro-5-trifluoromethyl-phenyl, 3-bromo-5-trifluoromethyl-phenyl, 3,5-dibromo-phenyl, 3,5-dichloro-phenyl, 3,4-dichloro-phenyl, 3-trifluoromethyl-phenyl, 4-bromo-3,5-dichlorophenyl, 4-fluoro-3,5-dichlorophenyl or 3,4,5-trichloro-phenyl, 3,5-dichloro-4-fluoro-phenyl; especially R⁴ is 3,5-dibromo-phenyl, 3,5-dichloro-phenyl, 3,5-bis-(trifluoromethyl)-phenyl, 3-chloro-5-trifluoromethyl-phenyl, 3-bromo-5-trifluoromethyl-phenyl, 4-bromo-3,5-dichlorophenyl, 3,4,5-trichloro-phenyl, 3,5-dichloro-4-fluoro-phenyl, pyridyl, or 3-5-substituted pyridyl wherein the substituents are selected from the group consisting of bromo, chloro, fluoro and trifluoromethyl.

Embodiment E34 of formula (I) provides compounds of formula (I) wherein A², A³ and A⁴ are each CH; R¹ is methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl; R³ is chlorodifluoromethyl or trifluoromethyl; A¹ is CR⁵, wherein R⁵ is chloro, bromo, trifluoromethyl, fluoro, or methyl; R² is selected from methyl, ethyl, CH₂F, dimethylamino, CHF₂, fluorine, cyclopropyl or 1-cyanocyclopropyl, and R⁴ is phenyl, pyridyl, mono-, di- or tri-substituted phenyl where the substituents are independently selected from the group consisting of chloro, bromo, fluoro and trifluoromethyl, or mono- or di-substituted pyridyl where the substituents are independently selected from chloro, bromo, fluoro and trifluoromethyl.

Embodiment E35 of formula (I) provides compounds of formula (I) wherein A², A³ and A⁴ are each CH; R¹ is methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl; R³ is trifluoromethyl; A¹ is CR⁵, wherein R⁵ is chloro or methyl; R² is independently selected from methyl, ethyl, CH₂F, dimethylamino, CHF₂, fluorine, cyclopropyl or 1-cyanocyclopropyl; and R⁴ is 3,5-dichloro-4-fluoro-phenyl, 3,5-dichloro-phenyl, 3,4,5-trichloro-phenyl, 3-bromo-5-trifluoromethyl-phenyl, 3-trifluoromethyl-phenyl or 3-chloro-5-trifluoromethyl-phenyl.

Embodiment E36 of formula (I) provides compounds of formula (I) wherein A², A³ and A⁴ are each CH; R¹ is methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl; R³ is trifluoromethyl; A¹ is CR⁵, wherein R⁵ is chloro; R² is selected from methyl, ethyl, CH₂F, dimethylamino, CHF₂, fluorine, cyclopropyl or 1-cyanocyclopropyl; and R⁴ is 3,5-bis-(trifluoromethyl)-phenyl, 3-chloro-5-trifluoromethyl-phenyl, 3-bromo-5-trifluoromethyl-phenyl, 3,5-dibromo-phenyl, 3,5-dichloro-phenyl, 3,4-dichloro-phenyl, 3-trifluoromethyl-phenyl, 4-bromo-3,5-dichlorophenyl, 4-fluoro-3,5-dichlorophenyl or 3,4,5-trichloro-phenyl, 3,5-dichloro-4-fluoro-phenyl; especially R⁴ is 3,5-dibromo-phenyl, 3,5-dichloro-phenyl, 3,5-bis-(trifluoromethyl)-phenyl, 3-chloro-5-trifluoromethyl-phenyl, 3-bromo-5-trifluoromethyl-phenyl, 4-bromo-3,5-dichlorophenyl, 3,4,5-trichloro-phenyl, 3,5-dichloro-4-fluoro-phenyl, pyridyl, or 3-5-substituted pyridyl wherein the substituents are selected from the group consisting of bromo, chloro, fluoro and trifluoromethyl.

Embodiment E37 of formula (I) provides compounds of formula (I) wherein A², A³ and A⁴ are each CH; R¹ is methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl; R³ is chlorodifluoromethyl or trifluoromethyl; A¹ is CR⁵, wherein R⁵ is chloro; R² is selected from methyl, ethyl, CH₂F, dimethylamino, CHF₂, fluorine, cyclopropyl or 1-cyanocyclopropyl, and R⁴ is phenyl, pyridyl, mono-, di- or tri-substituted phenyl where the substituents are independently selected from the group consisting of chloro, bromo, fluoro and trifluoromethyl, or mono- or di-substituted pyridyl where the substituents are independently selected from chloro, bromo, fluoro and trifluoromethyl.

Embodiment E38 of formula (I) provides compounds of formula (I) wherein A², A³ and A⁴ are each CH; R¹ is methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl; R³ is trifluoromethyl; A¹ is CR⁵, wherein R⁵ is chloro; R² is independently selected from methyl, ethyl, CH₂F, dimethylamino, CHF₂, fluorine, cyclopropyl or 1-cyanocyclopropyl; and R⁴ is 3,5-dichloro-4-fluoro-phenyl, 3,5-dichloro-phenyl, 3,4,5-trichloro-phenyl, 3-bromo-5-trifluoromethyl-phenyl, 3-trifluoromethyl-phenyl or 3-chloro-5-trifluoromethyl-phenyl.

Embodiment E39 of formula (I) provides compounds of formula (I) wherein A², A³ and A⁴ are each CH; R¹ is methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl; R³ is trifluoromethyl; A¹ is CR⁵, wherein R⁵ is bromo; R² is selected from methyl, ethyl, CH₂F, dimethylamino, CHF₂, fluorine, cyclopropyl or 1-cyanocyclopropyl; and R⁴ is 3,5-bis-(trifluoromethyl)-phenyl, 3-chloro-5-trifluoromethyl-phenyl, 3-bromo-5-trifluoromethyl-phenyl, 3,5-dibromo-phenyl, 3,5-dichloro-phenyl, 3,4-dichloro-phenyl, 3-trifluoromethyl-phenyl, 4-bromo-3,5-dichlorophenyl, 4-fluoro-3,5-dichlorophenyl or 3,4,5-trichloro-phenyl, 3,5-dichloro-4-fluoro-phenyl; especially R⁴ is 3,5-dibromo-phenyl, 3,5-dichloro-phenyl, 3,5-bis-(trifluoromethyl)-phenyl, 3-chloro-5-trifluoromethyl-phenyl, 3-bromo-5-trifluoromethyl-phenyl, 4-bromo-3,5-dichlorophenyl, 3,4,5-trichloro-phenyl, 3,5-dichloro-4-fluoro-phenyl, pyridyl, or 3-5-substituted pyridyl wherein the substituents are selected from the group consisting of bromo, chloro, fluoro and trifluoromethyl.

Embodiment E40 of formula (I) provides compounds of formula (I) wherein A², A³ and A⁴ are each CH; R¹ is methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl; R³ is chlorodifluoromethyl or trifluoromethyl; A¹ is CR⁵, wherein R⁵ is bromo; R² is selected from methyl, ethyl, CH₂F, dimethylamino, CHF₂, fluorine, cyclopropyl or 1-cyanocyclopropyl, and R⁴ is phenyl, pyridyl, mono-, di- or tri-substituted phenyl where the substituents are independently selected from the group consisting of chloro, bromo, fluoro and trifluoromethyl, or mono- or di-substituted pyridyl where the substituents are independently selected from chloro, bromo, fluoro and trifluoromethyl.

Embodiment E41 of formula (I) provides compounds of formula (I) wherein A², A³ and A⁴ are each CH; R¹ is methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl; R³ is trifluoromethyl; A¹ is CR⁵, wherein R⁵ is bromo; R² is independently selected from methyl, ethyl, CH₂F, dimethylamino, CHF₂, fluorine, cyclopropyl or 1-cyanocyclopropyl; and R⁴ is 3,5-dichloro-4-fluoro-phenyl, 3,5-dichloro-phenyl, 3,4,5-trichloro-phenyl, 3-bromo-5-trifluoromethyl-phenyl, 3-trifluoromethyl-phenyl or 3-chloro-5-trifluoromethyl-phenyl.

Embodiment E42 of formula (I) provides compounds of formula (I) wherein A², A³ and A⁴ are each CH; R¹ is methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl; R³ is trifluoromethyl; A¹ is CR⁵, wherein R⁵ is trifluoromethyl; R² is selected from methyl, ethyl, CH₂F, dimethylamino, CHF₂, fluorine, cyclopropyl or 1-cyanocyclopropyl; and R⁴ is 3,5-bis-(trifluoromethyl)-phenyl, 3-chloro-5-trifluoromethyl-phenyl, 3-bromo-5-trifluoromethyl-phenyl, 3,5-dibromo-phenyl, 3,5-dichloro-phenyl, 3,4-dichloro-phenyl, 3-trifluoromethyl-phenyl, 4-bromo-3,5-dichlorophenyl, 4-fluoro-3,5-dichlorophenyl or 3,4,5-trichloro-phenyl, 3,5-dichloro-4-fluoro-phenyl; especially R⁴ is 3,5-dibromo-phenyl, 3,5-dichloro-phenyl, 3,5-bis-(trifluoromethyl)-phenyl, 3-chloro-5-trifluoromethyl-phenyl, 3-bromo-5-trifluoromethyl-phenyl, 4-bromo-3,5-dichlorophenyl, 3,4,5-trichloro-phenyl, 3,5-dichloro-4-fluoro-phenyl, pyridyl, or 3-5-substituted pyridyl wherein the substituents are selected from the group consisting of bromo, chloro, fluoro and trifluoromethyl.

Embodiment E43 of formula (I) provides compounds of formula (I) wherein A², A³ and A⁴ are each CH; R¹ is methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl; R³ is chlorodifluoromethyl or trifluoromethyl; A¹ is CR⁵, wherein R⁵ is trifluoromethyl; R² is selected from methyl, ethyl, CH₂F, dimethylamino, CHF₂, fluorine, cyclopropyl or 1-cyanocyclopropyl, and R⁴ is phenyl, pyridyl, mono-, di- or tri-substituted phenyl where the substituents are independently selected from the group consisting of chloro, bromo, fluoro and trifluoromethyl, or mono- or di-substituted pyridyl where the substituents are independently selected from chloro, bromo, fluoro and trifluoromethyl.

Embodiment E44 of formula (I) provides compounds of formula (I) wherein A², A³ and A⁴ are each CH; R¹ is methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl; R³ is trifluoromethyl; A¹ is CR⁵, wherein R⁵ is trifluoromethyl; R² is independently selected from methyl, ethyl, CH₂F, dimethylamino, CHF₂, fluorine, cyclopropyl or 1-cyanocyclopropyl; and R⁴ is 3,5-dichloro-4-fluoro-phenyl, 3,5-dichloro-phenyl, 3,4,5-trichloro-phenyl, 3-bromo-5-trifluoromethyl-phenyl, 3-trifluoromethyl-phenyl or 3-chloro-5-trifluoromethyl-phenyl.

Embodiment E45 of formula (I) provides compounds of formula (I) wherein A², A³ and A⁴ are each CH; R¹ is methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl; R³ is trifluoromethyl; A¹ is CR⁵, wherein R⁵ is methyl; R² is selected from methyl, ethyl, CH₂F, dimethylamino, CHF₂, fluorine, cyclopropyl or 1-cyanocyclopropyl; and R⁴ is 3,5-bis-(trifluoromethyl)-phenyl, 3-chloro-5-trifluoromethyl-phenyl, 3-bromo-5-trifluoromethyl-phenyl, 3,5-dibromo-phenyl, 3,5-dichloro-phenyl, 3,4-dichloro-phenyl, 3-trifluoromethyl-phenyl, 4-bromo-3,5-dichlorophenyl, 4-fluoro-3,5-dichlorophenyl or 3,4,5-trichloro-phenyl, 3,5-dichloro-4-fluoro-phenyl; especially R⁴ is 3,5-dibromo-phenyl, 3,5-dichloro-phenyl, 3,5-bis-(trifluoromethyl)-phenyl, 3-chloro-5-trifluoromethyl-phenyl, 3-bromo-5-trifluoromethyl-phenyl, 4-bromo-3,5-dichlorophenyl, 3,4,5-trichloro-phenyl, 3,5-dichloro-4-fluoro-phenyl, pyridyl, or 3-5-substituted pyridyl wherein the substituents are selected from the group consisting of bromo, chloro, fluoro and trifluoromethyl.

Embodiment E46 of formula (I) provides compounds of formula (I) wherein A², A³ and A⁴ are each CH; R¹ is methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl; R³ is chlorodifluoromethyl or trifluoromethyl; A¹ is CR⁵, wherein R⁵ is methyl; R² is selected from methyl, ethyl, CH₂F, dimethylamino, CHF₂, fluorine, cyclopropyl or 1-cyanocyclopropyl, and R⁴ is phenyl, pyridyl, mono-, di- or tri-substituted phenyl where the substituents are independently selected from the group consisting of chloro, bromo, fluoro and trifluoromethyl, or mono- or di-substituted pyridyl where the substituents are independently selected from chloro, bromo, fluoro and trifluoromethyl.

Embodiment E47 of formula (I) provides compounds of formula (I) wherein A², A³ and A⁴ are each CH; R¹ is methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl; R³ is trifluoromethyl; A¹ is CR⁵, wherein R⁵ is methyl; R² is independently selected from methyl, ethyl, CH₂F, dimethylamino, CHF₂, fluorine, cyclopropyl or 1-cyanocyclopropyl; and R⁴ is 3,5-dichloro-4-fluoro-phenyl, 3,5-dichloro-phenyl, 3,4,5-trichloro-phenyl, 3-bromo-5-trifluoromethyl-phenyl, 3-trifluoromethyl-phenyl or 3-chloro-5-trifluoromethyl-phenyl.

In one embodiment the invention provides compounds of formula (IA)

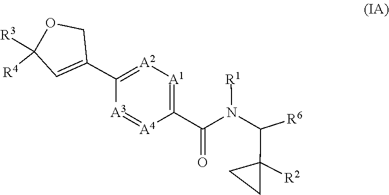

wherein A¹, A², A³, A⁴, R¹, R², R³ and R⁴ and their preferred values are as defined for a compound of formula (I); or a salt or N-oxide thereof. Further preferred in this embodiment are the embodiments E1 to 47.

In a further embodiment the invention provides compounds of formula (IB)

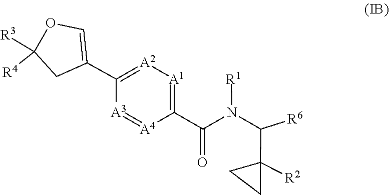

wherein A¹, A², A³, A⁴, R¹, R², R³ and R⁴ and their preferred values are as defined for a compound of formula (I); or a salt or N-oxide thereof. Further preferred in this embodiment are the embodiments E1 to 47.

In a further embodiment the invention provides compounds of formula (IC)

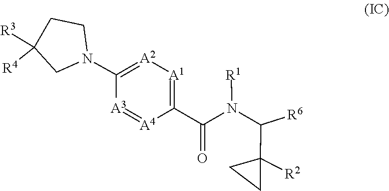

wherein A¹, A², A³, A⁴, R¹, R², R³ and R⁴ and their preferred values are as defined for a compound of formula (I); or a salt or N-oxide thereof. Further preferred in this embodiment are the embodiments E1 to E47.

In a further embodiment the invention provides compounds of formula (ID)

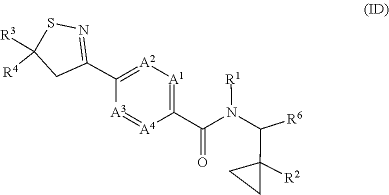

wherein A¹, A², A³, A⁴, R¹, R², R³ and R⁴ and their preferred values are as defined for a compound of formula (I); or a salt or N-oxide thereof. Further preferred in this embodiment are the embodiments E1 to E47.

In a further embodiment the invention provides compounds of formula (IE)

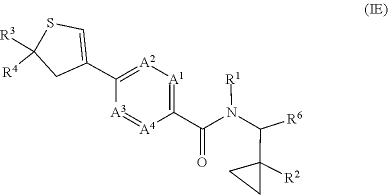

wherein A¹, A², A³, A⁴, R¹, R², R³ and R⁴ and their preferred values are as defined for a compound of formula (I); or a salt or N-oxide thereof. Further preferred in this embodiment are the embodiments E1 to E47.

In a further embodiment the invention provides compounds of formula (IF)

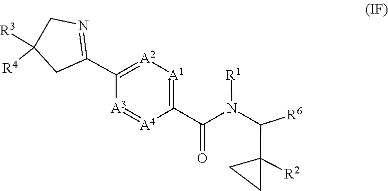

wherein A¹, A², A³, A⁴, R¹, R², R³ and R⁴ and their preferred values are as defined for a compound of formula (I); or a salt or N-oxide thereof. Further preferred in this embodiment are the embodiments E1 to E47.

In a further embodiment the invention provides compounds of formula (IG)

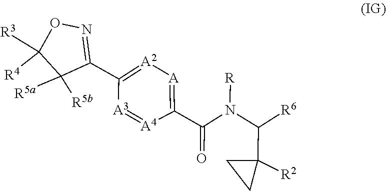

wherein A¹, A², A³, A⁴, R¹, R², R³, R⁴, R^{5a} and R^{5b} and their preferred values are as defined for a compound of formula (I); or a salt or N-oxide thereof. Further preferred in this embodiment are the embodiments E1 to E47. Preferably R^{5a} is halogen, hydroxyl, C₁-C₈alkylthio-, C₁-C₈haloalkylthio-, C₁-C₈alkylsulfinyl-, C₁-C₈haloalkylsulfinyl-, C₁-C₈alkylsulfonyl-, C₁-C₈haloalkylsulfonyl-, C₁-C₈alkyl, C₂-C₈alkenyl, C₂-C₈alkynyl, C₁-C₈haloalkyl, C₂-C₈haloalkenyl, most preferably halogen, C₁-C₈alkylthio- or C₁-C₈alkyl. Preferably R^{5b} is halogen or hydrogen.

In a further embodiment the invention provides compounds of formula (IH)

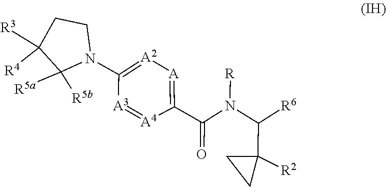

wherein A¹, A², A³, A⁴, R¹, R², R³ and R⁴ and their preferred values are as defined for a compound of formula (I); or a salt or N-oxide thereof. Further preferred in this embodiment are the embodiments E1 to E47.

In a further embodiment the invention provides compounds of formula (IJ)

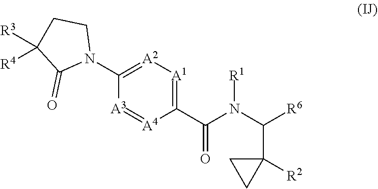

wherein A¹, A², A³, A⁴, R¹, R², R³ and R⁴ and their preferred values are as defined for a compound of formula (I); or a salt or N-oxide thereof. Further preferred in this embodiment are the embodiments E1 to E47.

The present invention also provides intermediates useful for the preparation of compounds of formula (I). Certain intermediates are novel and as such form a further aspect of the invention. One group of novel intermediates are compounds of formula (Int-1)

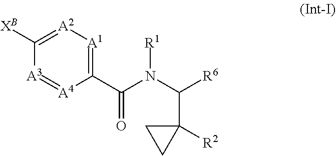

wherein A¹, A², A³, A⁴, R¹ and R² are as defined for a compound of formula (I) and X^{B} is a halogen, such as bromo, or X^{B} is cyano, formyl, CH═N—OH or acetyl; or a salt or N-oxide thereof. The preferences for A¹, A², A³, A⁴, R¹ and R² are the same as the preferences set out for the corresponding substituents of a compound of formula (I).

Another group of novel intermediates are compounds of formula (Int-II)

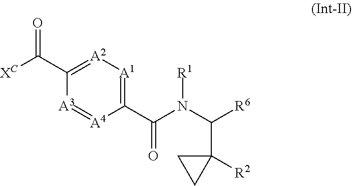

wherein A¹, A², A³, A⁴, R¹ and R² are as defined for a compound of formula (I); X^{C} is CH₂-halogen, wherein halogen is preferably bromo or chloro, CH═C(R³)R⁴ or CH₂C(OH)(R³)R⁴ wherein R³ and R⁴ are as defined for a compound of formula (I); or a salt or N-oxide thereof. The preferences for A¹, A², A³, A⁴, R¹ and R² are the same as the preferences set out for the corresponding substituents of a compound of formula (I).

Another group of novel intermediates are compounds of formula (Int-III)

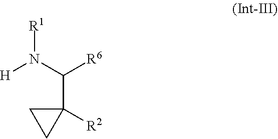

wherein R¹ and R² are as defined for a compound of formula (I); or a salt or N-oxide thereof. The preferences for R¹ and R² are the same as the preferences set out for the corresponding substituents of a compound of formula (I).

Another group of novel intermediates are compounds of formula (Int-V)

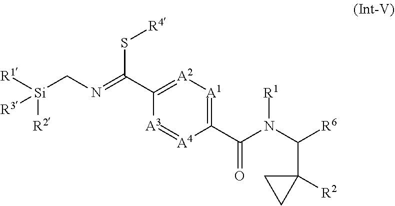

wherein R^{1′}, R^{2′} and R^{3′} are independently of each other optionally substituted alkyl or optionally substituted phenyl, R^{4′} is optionally substituted alkyl, and A¹, A², A³, A⁴, R¹ and R² are as defined for a compound of formula (I); or a salt or N-oxide thereof. The preferences for A¹, A², A³, A⁴, R¹ and R² are the same as the preferences set out for the corresponding substituents of a compound of formula (I). For example, the preferences for A¹, A², A³, A⁴, R¹ and R² may be the same as for formula (IA), (IB), (IC), (ID), (IE), (IF), (IG), (IH), (IJ). Preferably R^{1′}, R^{2′} and R^{3′} are each independently C₁-C₈alkyl or phenyl. Preferably R^{4′} is C₁-C₈ alkyl.

Another group of novel intermediates are compounds of formula (Int-VI)

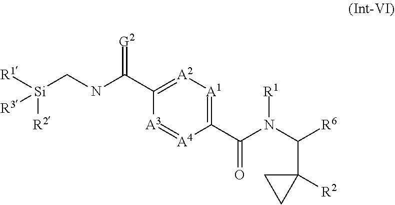

wherein G² is O or S, R^{1′}, R^{2′} and R^{3′} are independently of each other optionally substituted alkyl or optionally substituted phenyl, and A¹, A², A³, A⁴, R¹ and R² are as defined for a compound of formula (I); or a salt or N-oxide thereof. The preferences for A¹, A², A³, A⁴, R¹ and R² are the same as the preferences set out for the corresponding substituents of a compound of formula (I). For example, the preferences for A¹, A², A³, A⁴, R¹ and R² may be the same as for formula (IA), (IB), (IC), (ID), (IE), (IF), (IG), (IH), (IJ), (IK), (IL) and (IM). Preferably R^{1′}, R^{2′} and R^{3′} are each independently C₁-C₈alkyl or phenyl. Preferably R^{4′} is C₁-C₈ alkyl.

Another group of novel intermediates are compounds of formula (Int-VII)

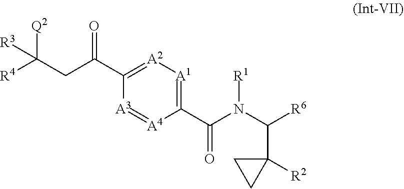

wherein Q² is CH₂—NO₂, CN or group Qa

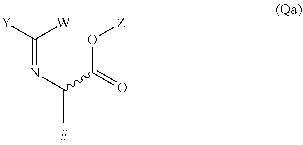

W is hydrogen or optionally substituted aryl, Y is optionally substituted aryl, and Z is optionally substituted alkyl or optionally substituted arylalkylene, and A¹, A², A³, A⁴, R¹, R², R³ and R⁴ are as defined for a compound of formula (I); or a salt or N-oxide thereof. The preferences for A¹, A², A³, A⁴, R¹, R², R³ and R⁴ are the same as the preferences set out for the corresponding substituents of a compound of formula (I). For example, the preferences for A¹, A², A³, A⁴, R¹, R², R³ and R⁴ may be the same as for formula (IA), (IB), (IC), (ID), (IE), (IF), (IG), (IH), (IJ), (IK), (IL) and (IM). Preferably W is hydrogen or phenyl. Preferably Y is phenyl. Preferably Z is C₁-C₈alkyl, or phenyl-C₁-C₈alkyl.

Another group of novel intermediates are compounds of formula (Int-VIII)

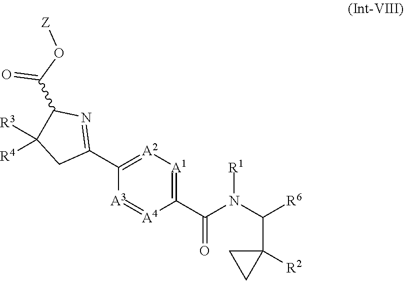

wherein Z is optionally substituted alkyl or optionally substituted arylalkylene, and A¹, A², A³, A⁴, R¹, R², R³ and R⁴ are as defined for a compound of formula (I); or a salt or N-oxide thereof. The preferences for A¹, A², A³, A⁴, R¹, R², R³ and R⁴ are the same as the preferences set out for the corresponding substituents of a compound of formula (I). For example, the preferences for A¹, A², A³, A⁴, R¹, R², R³ and R⁴ may be the same as for formula (IA), (IB), (IC), (ID), (IE), (IF), (IG), (IH), (IJ), (IK), (IL) and (IM). Preferably Z is C₁-C₈alkyl, or phenyl-C₁-C₈alkyl.

Another group of novel intermediates are compounds of formula (Int-IX)

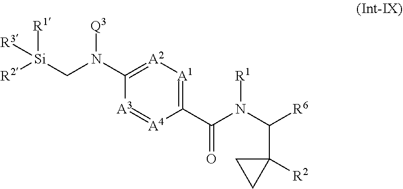

wherein Q³ is CH₂—OR^{4′} or CH₂—CN, R^{1′}, R^{2′} and R^{3′} are independently of each other optionally substituted alkyl or optionally substituted phenyl, R^{4′} is optionally substituted alkyl, and A¹, A², A³, A⁴, R¹ and R² are as defined for a compound of formula (I); or a salt or N-oxide thereof. The preferences for A¹, A², A³, A⁴, R¹ and R² are the same as the preferences set out for the corresponding substituents of a compound of formula (I). For example, the preferences for A¹, A², A³, A⁴, R¹ and R² may be the same as for formula (IA), (IB), (IC), (ID), (IE), (IF), (IG), (IH), (IJ), (IK), (IL) and (IM). Preferably R^{1′}, R^{2′} and R^{3′} are each independently C₁-C₈alkyl or phenyl. Preferably R^{4′} is C₁-C₈ alkyl.

Another group of novel intermediates are compounds of formula (Int-X)

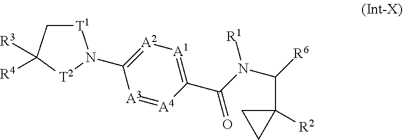

wherein T¹ and T² are independently CH₂ or C═O or CHOH, providing that at least one of T¹ and T² is C═O or CHOH, and A¹, A², A³, A⁴, R¹, R², R³ and R⁴ are as defined for a compound of formula (I); or a salt or N-oxide thereof. The preferences for A¹, A², A³, A⁴, R¹, R², R³ and R⁴ are the same as the preferences set out for the corresponding substituents of a compound of formula (I). For example, the preferences for A¹, A², A³, A⁴, R¹, R², R³ and R⁴ may be the same as for formula (IA), (IB), (IC), (ID), (IE), (IF), (IG), (IH), (IJ), (IK), (IL) and (IM).

Compounds of formula (I) include at least one chiral center and may exist as compounds of formula (I*) or compounds of formula (I**):

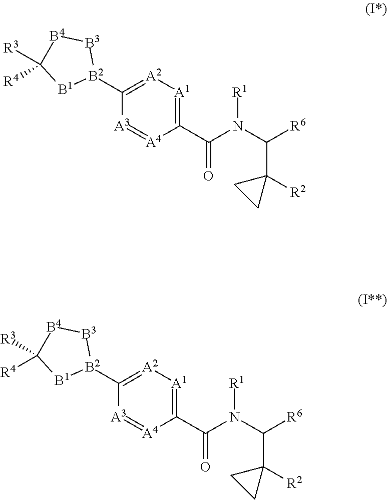

Generally compounds of formula (I**) are more biologically active than compounds of formula (I*). The invention includes mixtures of compounds (I*) and (I**) in any ratio e.g. in a molar ratio of 1:99 to 99:1, e.g. 10:1 to 1:10, e.g. a substantially 50:50 molar ratio. In an enantiomerically (or epimerically) enriched mixture of formula (I**), the molar proportion of compound (I**) compared to the total amount of both enantiomers (or epimers) is for example greater than 50%, e.g. at least 55, 60, 65, 70, 75, 80, 85, 90, 95, 96, 97, 98, or at least 99%. Likewise, in enantiomerically (or epimerically) enriched mixture of formula (I*), the molar proportion of the compound of formula (I*) compared to the total amount of both enantiomers (or epimers) is for example greater than 50%, e.g. at least 55, 60, 65, 70, 75, 80, 85, 90, 95, 96, 97, 98, or at least 99%. Enantiomerically (or epimerically) enriched mixtures of formula (I**) are preferred.

The compounds of the invention may be made by a variety of methods as shown in Schemes 1 to 20.

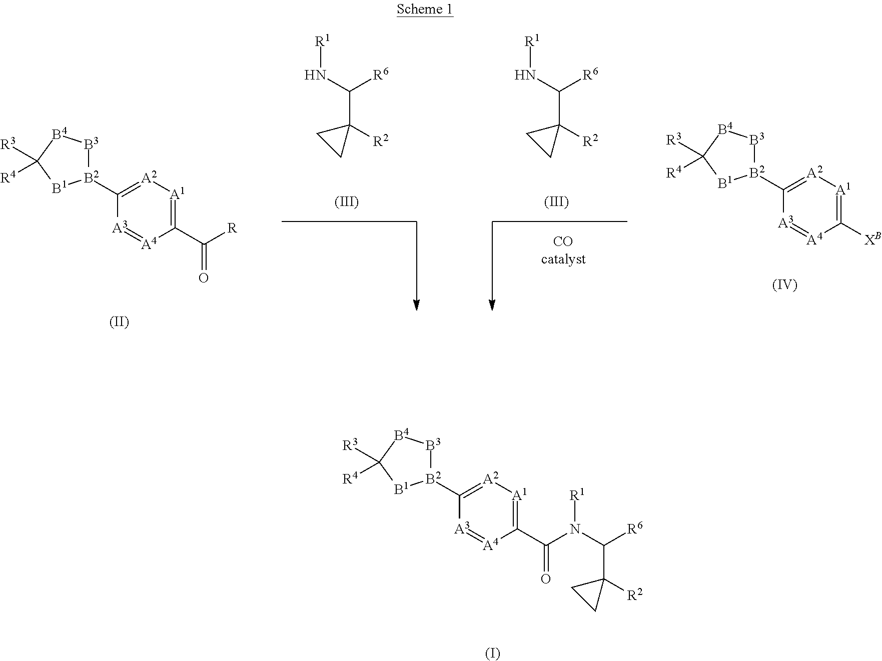

1) Compounds of formula (I) can be prepared by reacting a compound of formula (II) wherein R is OH, C₁-C₆alkoxy or Cl, F or Br, with an amine of formula (III), as shown in Scheme 1. When R is OH such reactions are usually carried out in the presence of a coupling reagent, such as N,N′-dicyclohexylcarbo-diimide (“DCC”), 1-ethyl-3-(3-dimethylamino-propyl)carbodiimide hydrochloride (“EDC”) or bis(2-oxo-3-oxazolidinyl)phosphonic chloride (“BOP-Cl”), in the presence of a base, and optionally in the presence of a nucleophilic catalyst, such as hydroxybenzotriazole (“HOBT”). When R is Cl, such reactions are usually carried out in the presence of a base, and optionally in the presence of a nucleophilic catalyst.

Alternatively, it is possible to conduct the reaction in a biphasic system comprising an organic solvent, preferably ethyl acetate, and an aqueous solvent, preferably a solution of sodium hydrogen carbonate. When R is C₁-C₆alkoxy it is sometimes possible to convert the ester directly to the amide by heating the ester and amine together in a thermal process. Suitable bases include pyridine, triethylamine, 4-(dimethylamino)-pyridine (“DMAP”) or diisopropylethylamine (Hunig's base). Preferred solvents are N,N-dimethylacetamide, tetrahydrofuran, dioxane, 1,2-dimethoxyethane, ethyl acetate and toluene. The reaction is carried out at a temperature of from 0° C. to 100° C., preferably from 15° C. to 30° C., in particular at ambient temperature. Amines of formula (III) can be prepared using method 11 described below.

2) Acid halides of formula (II), wherein R is Cl, F or Br, may be made from carboxylic acids of formula (II), wherein R is OH, under standard conditions, as described for example in WO09080250.

3) Carboxylic acids of formula (II), wherein R is OH, may be formed from esters of formula (II), wherein R is C₁-C₆alkoxy as described for example in WO09080250.

4) Compounds of formula (I) can be prepared by reacting a compound of formula (IV) wherein X^{B} is a leaving group, for example a halogen, such as bromo, with carbon monoxide and an amine of formula (III), in the presence of a catalyst, such as palladium(II) acetate or bis(triphenylphosphine)palladium(II) dichloride, optionally in the presence of a ligand, such as triphenylphosphine, and a base, such as sodium carbonate, pyridine, triethylamine, 4-(dimethylamino)-pyridine (“DMAP”) or diisopropylethylamine (Hunig's base), in a solvent, such as water, N,N-dimethylformamide or tetrahydrofuran. The reaction is carried out at a temperature of from 50° C. to 200° C., preferably from 100° C. to 150° C. The reaction is carried out at a pressure of from 50 to 200 bar, preferably from 100 to 150 bar.

5) Compounds of formula (IV) wherein X^{B} is a leaving group, for example a halogen, such as bromo, can be made by a various of methods, for example as described in WO09080250.

In all the following schemes, A¹, A², A³ and A⁴ are as described for compounds of formula (I), and P can be a leaving group, for example a halogen, such as bromo, iodo, chloro or described by one of the three groups A and B:

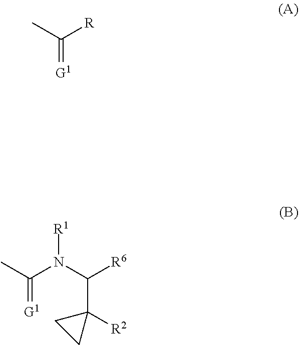

wherein G¹ is oxygen and R is OH, C₁-C₁₂alkoxy or Cl, F or Br, and R¹ and R² are as described for compounds of formula (I).

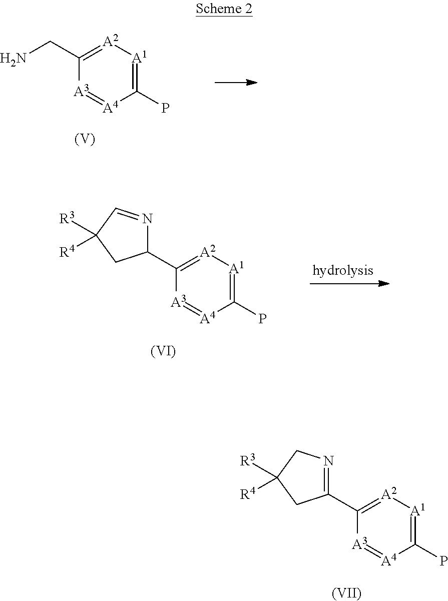

7) Compounds of formula (VII) can be prepared by various methods from an intermediate of formula (VI) as shown in Scheme 2 according to similar methods to those described in WO10149506. An intermediate of formula (VI) can be prepared for example from an intermediate of formula (V) as described in the same reference.

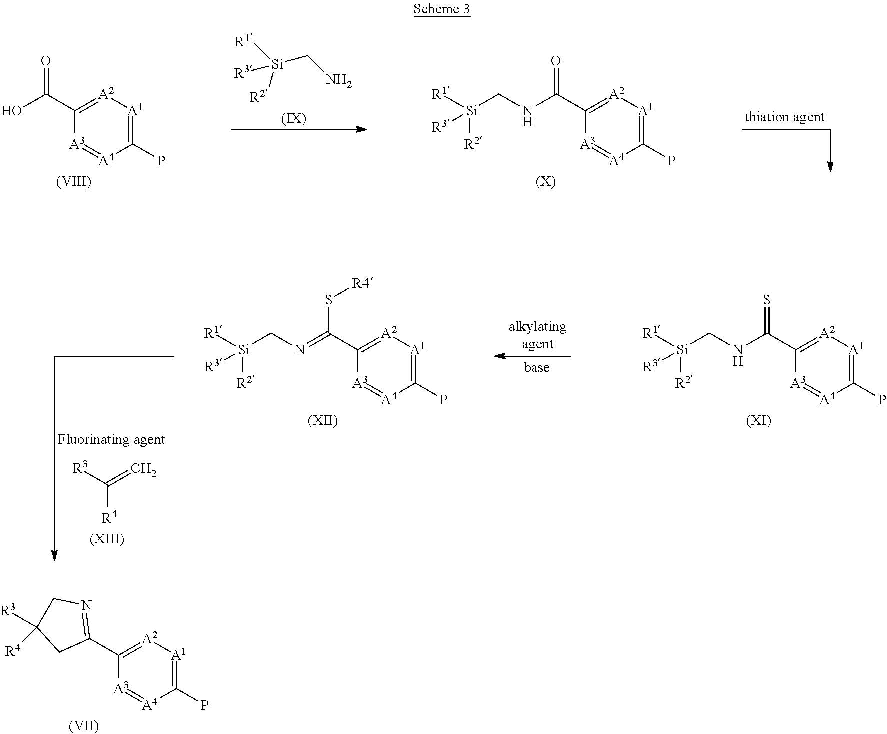

8) Alternatively, compounds of formula (VII) can be prepared by various methods from an intermediate of formula (XII) as shown in Scheme 3 according to similar methods to those described in WO10149506. The intermediates of formula (XII) can be prepared for example as described in the same reference.

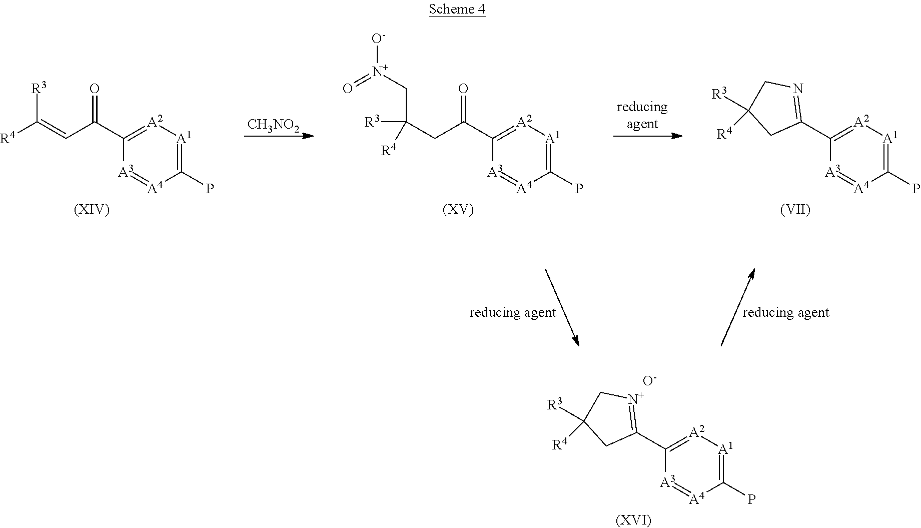

9) Alternatively, compounds of formula (VII) can be prepared by various methods from an intermediate of formula (XV) or (XVI) as shown in Scheme 4 according to similar methods to those described in WO10149506. The intermediates of formula (XV) can be prepared for example as described in the same reference.

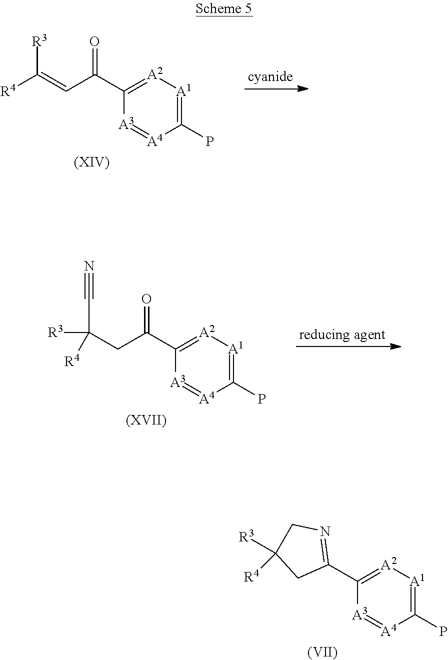

10) Alternatively, compounds of formula (VII) can be prepared by various methods from an intermediate of formula (XVII) as shown in Scheme 5 according to similar methods to those described in WO10149506. The intermediates of formula (XVII) can be prepared for example as described in the same reference.

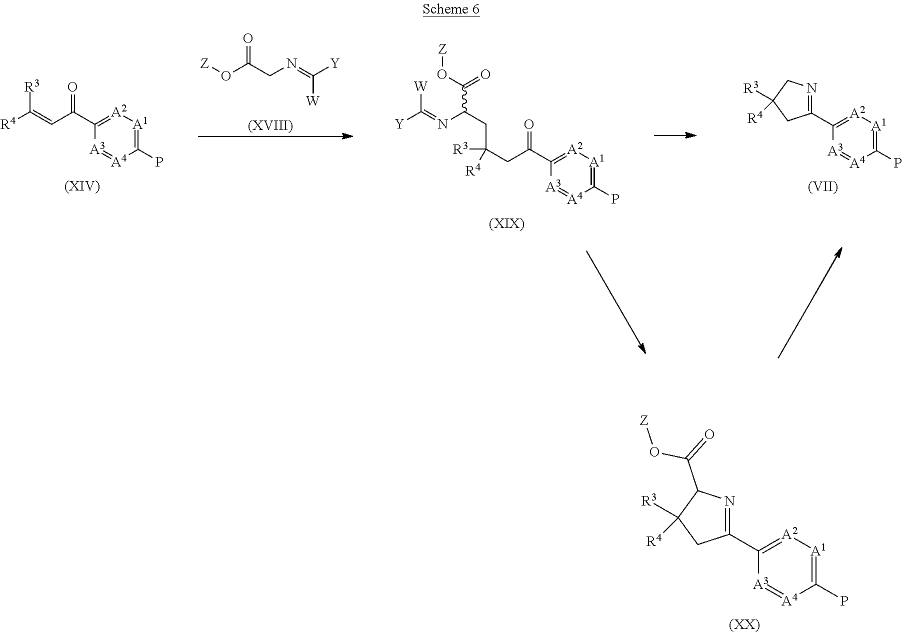

11) Compounds of formula (XIX) wherein R³ and R⁴ are as defined for the compound of formula I, and wherein W is hydrogen or optionally substituted aryl, Y is optionally substituted aryl, and Z is optionally substituted alkyl or optionally substituted arylalkylene, can be prepared by reacting a compound of formula (XIV) with a glycine Schiff base of formula (XVIII), in the presence of base. In most cases it is advantageous to conduct the reaction using a solvent at a dilution of 0.1 M to 1 M, preferably 0.3 M to 0.5 M. Suitable organic solvents could be used, for example toluene, 1,2-dichloroethane, dichloromethane, tetrahydrofuran, methanol or ethyl acetate. The reaction temperature is usually between 0° C. to 100° C., preferably between 40 and 100° C. When a solvent is used the reactants are usually at a dilution of e.g. between 0.1 M to 1 M. The reaction time is usually between 0 and 96 hours, preferably between 0 and 12 hours. Suitable bases include amines, such as triethylamine, 2,5-dimethylpiperazine, tetramethylpiperidine, 4-dimethylamino pyridine, potassium carbonate, metal alkoxides, such as sodium t-butoxide or metal fluorides, such as cesium fluoride.

12) Compounds of formula (XX) can be prepared by deprotecting and cyclizing compounds of formula (XIX). Suitable conditions for this transformation include acidic conditions, for instance the presence of strong acids such as trifluoroacetic acid, sulfonic acid or hydrochloric acid. Suitable solvents can be used, for example acetone, dimethylsulfoxide, dimethylformamide, toluene, xylenes, 1,2-dichloroethane, dichloromethane, tetrahydrofuran, methanol ethanol, tert-butanol, water or ethyl acetate at a temperature from 0° C. to 140° C., preferably between 0° C. and 80° C., and at dilution of e.g. between 0.1 M to 1 M. The reaction time is usually between 1 and 24 hours, preferably between 1 and 6 hours.

13) Alternatively, compounds of formula (VII) can be prepared by decarboxylating compounds of formula (XX). Suitable conditions for this transformation involve heating the compounds in a suitable media, which depending on the group Z may include some standard additives known by a person skilled in the art. Suitable solvents can be used, for example acetone, dimethylsulfoxide, dimethylformamide, toluene, xylenes, 1,2-dichloroethane, dichloromethane, tetrahydrofuran, methanol, ethanol, tert-butanol, water or ethyl acetate. The temperature is usually between 0° C. and 200° C., preferably between 50 and 180° C. Where a solvent is used, the reactants are usually at dilution of e.g. between 0.1 M to 1 M. The reaction time is usually between 0 and 96 hours, preferably between 0 and 24 hours. The reaction can also be performed under microwave conditions, preferably between 40 and 180° C., In some cases, however, it is necessary or useful to add an additive, such as a metal halide, for instance sodium chloride or potassium iodide, or a metal cyanide, such as sodium cyanide to the reaction media, or a base (e.g. when group Z is alkyl). In the case where Z is aryl-methylene (e.g. benzyl), suitable deprotection conditions include hydrogenation conditions. The most useful solvents are alcohols such as methanol or ethanol and in most cases it is advantageous to conduct the reaction at dilution between 0.1 M to 1 M, preferably 0.3 M to 0.5 M. The amount of catalyst, such as palladium on charcoal added is usually between 0.1 molar equivalent and 0.50 molar equivalents and the reaction time in most cases is between 1 hour and 6 hours. 14) Compounds of formula (VII) can be prepared by deprotecting, decarboxylating and cyclizing compounds of formula (XIX) according to a one-pot stepwise procedure without isolating the intermediates. Suitable conditions for this transformation include acidic conditions, for instance the presence of strong acids such as trifluoroacetic acid or hydrochloric acid, or basic conditions, depending on the group Z. Suitable solvents could be used, for example acetone, dimethylsulfoxide, dimethylformamide, toluene, xylenes, 1,2-dichloroethane, dichloromethane, tetrahydrofuran, methanol, ethanol, tert-butanol, water or ethyl acetate. The temperature is usually between 0° C. and 200° C., preferably between 50 and 180° C. Where a solvent is used the reactants are usually at dilution of e.g. between 0.1 M to 1 M. The reaction time is usually between land 96 hours, preferably between 1 and 12 hours. The reaction can also be performed under microwave conditions, preferably between 40 and 180° C. In some cases, however, it is necessary or useful to add an additive, such as a metal halide, for instance sodium chloride or potassium iodide, or a metal cyanide, such as sodium cyanide to the reaction media.

Enantiomerically enriched mixtures of compounds of formula (VII) may be prepared, for example, according to schemes 4, 5 or 6 by formation of intermediate XV, XVII or XIX via an asymmetric Michael addition, see for example J. Org. Chem. 2008, 73, 3475-3480 and references cited therein” and J. Am. Chem. Soc. 2008, 130, 6072-6073.

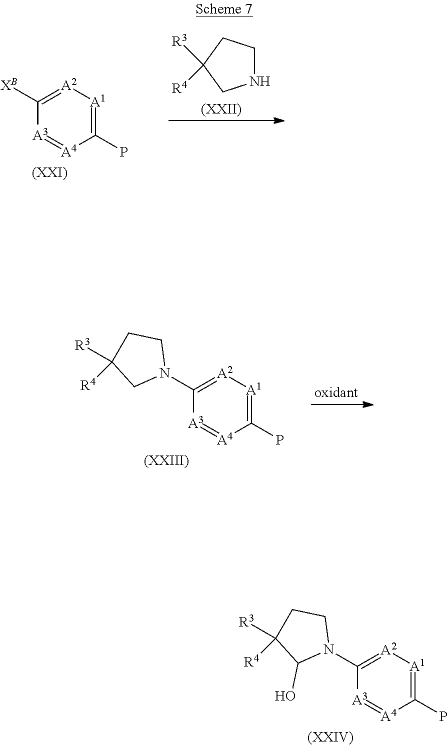

15) Compounds of formula (XXIII) can be prepared by reaction of compound of formula (XXI) wherein X^{B} is a halogen, such as bromine or chlorine, with an amine compound of formula (XXII) as shown in Scheme 7 in the absence or the presence of a catalyst, such as palladium(II) acetate or bis-(triphenylphosphine)palladium(II) dichloride, optionally in the presence of a ligand, such as triphenylphosphine, and a base, such as sodium carbonate, pyridine, triethylamine, 4-(dimethylamino)-pyridine (“DMAP”) or diisopropylethylamine (Hunig's base), in a solvent, such as water, N,N-dimethylformamide or tetrahydrofuran. The reaction is carried out at a temperature of from 50° C. to 200° C., preferably from 100° C. to 150° C. The reaction is carried out at a pressure of from 50 to 200 bar, preferably from 100 to 150 bar.

Compounds of formula (XXII) can be prepared according to a method developed in the literature: Tetrahedron (1996), 52, (1), 59-70 and Tetrahedron Letters (1993), 34, (20), 3279-82.

16) Compounds of formula (XXI) wherein X^{B} is a leaving group, for example a halogen, such as bromo, can be prepared as described in WO09080250.

17) Compounds of formula (XXIV) can be prepared by reaction of compound of formula (XXIII) with an oxidant, as described in WO12035011.

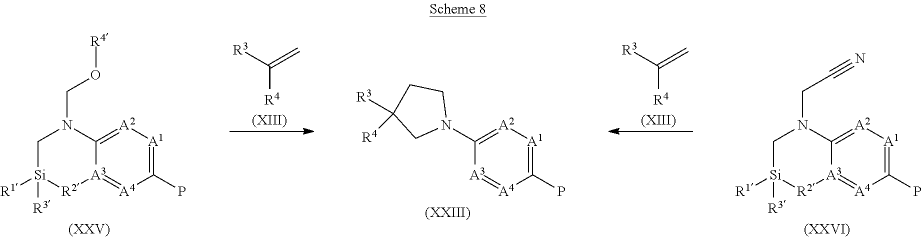

18) Compounds of formula (XXIII) can be prepared by reaction of compound of formula (XXVI) wherein R^{1′}, R^{2′} and R^{3′} are independently of each other optionally substituted alkyl or optionally substituted phenyl groups, with styrene of formula (XIII) according to a method developed in the literature: Journal of Medicinal Chemistry (1990), 33(2), 849-54.

19) Compounds of formula (XXIII) can be prepared by reaction of compound of formula (XXV) wherein R^{4′} is optionally substituted alkyl group, R^{1′}, R^{2′} and R^{3′} are independently of each other optionally substituted alkyl or optionally substituted phenyl groups, with styrene of formula (XIII) according to a method developed in the literature: Tetrahedron (1996), 52, (1), 59-70 and Tetrahedron Letters (1993), 34, (20), 3279-82.

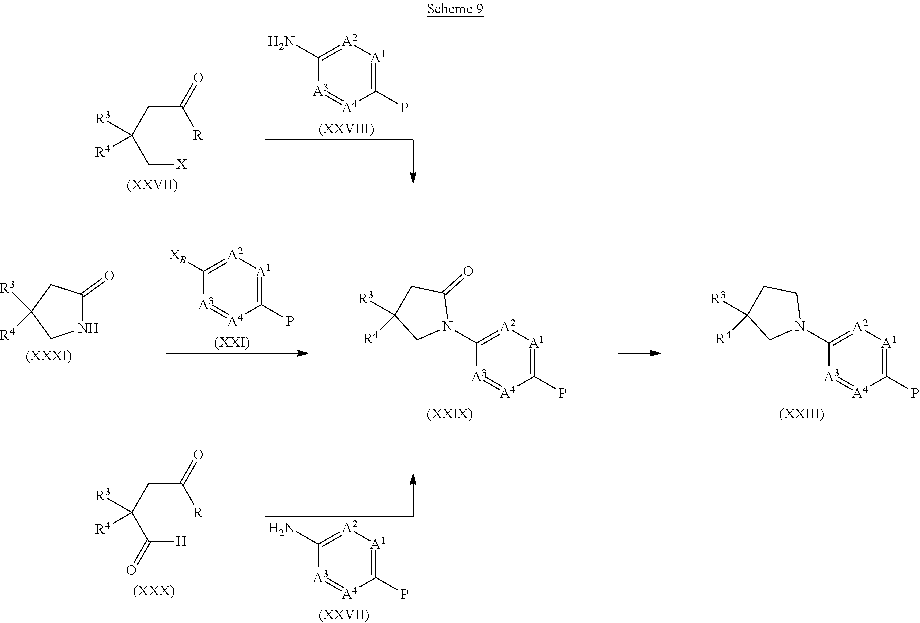

20) Compounds of formula (XXIII) can be prepared by reduction of compounds of formula (XXIX) with a metal hydride, for instance according to a method developed in the literature: Journal of Pharmaceutical Sciences (1978), 67(7), 953-6.

21) Compounds of formula (XXIX) can be prepared by reaction of compound of formula (XXXI) with a compound of formula (XXI) as described in 15).

Compounds of formula (XXXI) can be prepared by many methods as described in the literature (Bioorganic & Medicinal Chemistry Letters (2010), 20(1), 362-365).

22) Compounds of formula (XXIX) can be prepared by reaction of compound of formula (XXVII) wherein R is OH, C₁-C₆alkoxy or Cl, F or Br, with a compound of formula (XXVIII) under standard reductive amination conditions.

23) Compounds of formula (XXVII) can be prepared by many methods as described in the literature (US patent US 2005148792).

24) Compounds of formula (XXIX) can be prepared by reaction of compound of formula (XXVII) wherein R is OH, C₁-C₆alkoxy or Cl, F or Br, and X is a leaving group, such as a mesylate, a tosylate or an halogen with a compound of formula (XXVIII) under standard substitution reaction conditions.

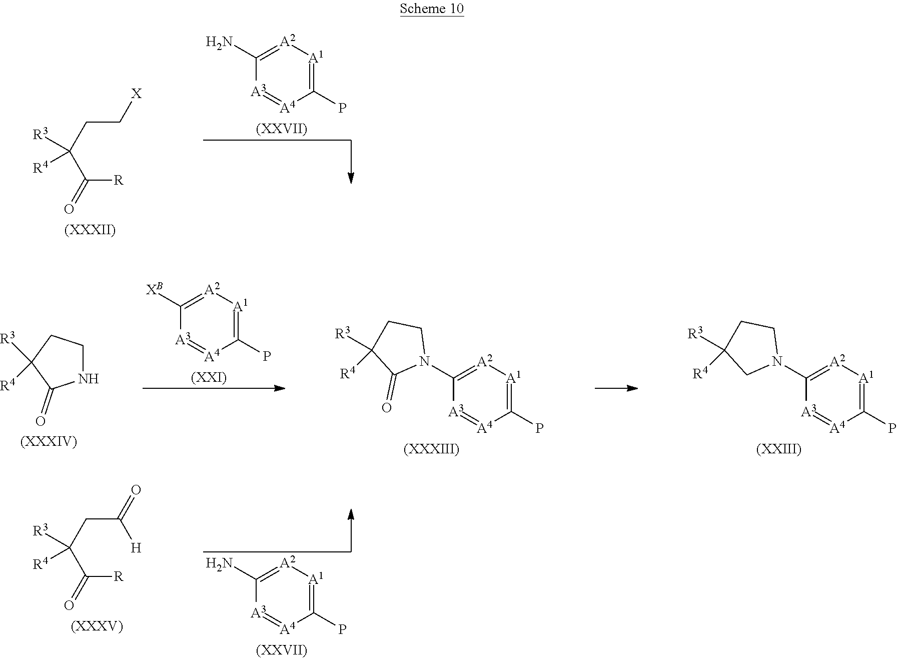

25) Compounds of formula (XXIII) can be prepared by reduction of compounds of formula (XXXIII) with a metal hydride, for instance according to a method developed in the literature: Tetrahedron: Asymmetry (1999), 10(20), 3877-3881

26) Compounds of formula (XXXIII) can be prepared by reaction of compound of formula (XXXIV) with a compound of formula (XXI) as described in 15).

27) Compounds of formula (XXXIV) can be prepared by many methods as described in the literature (Tetrahedron: Asymmetry (1999), 10(20), 3877-3881).

28) Compounds of formula (XXXIII) can be prepared by reaction of compound of formula (XXXV) wherein R is OH, C₁-C₆alkoxy or Cl, F or Br, with a compound of formula (XXVII) under standard reductive amination conditions.

29) Compounds of formula (XXXV) can be prepared by many methods as described in the literature.

30) Compounds of formula (XXXIV) can be prepared by reaction of compound of formula (XXXII) wherein R is OH, C₁-C₆alkoxy or Cl, F or Br, and X is a leaving group, such as a mesylate, a tosylate or an halogen with a compound of formula (XXVII) under standard substitution reaction conditions.

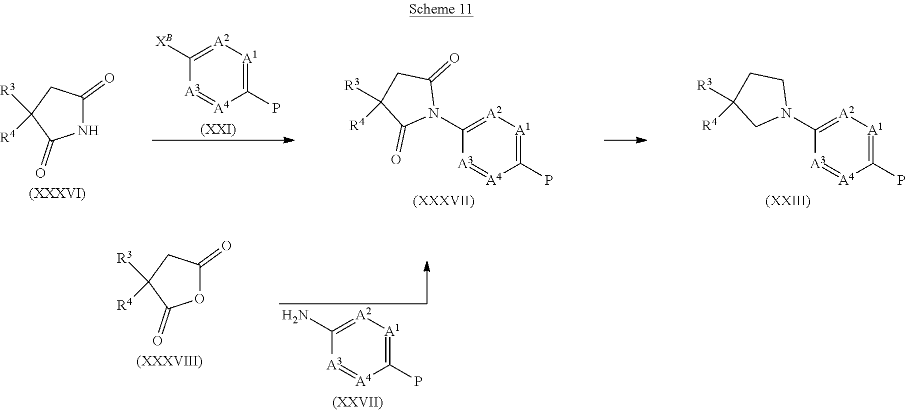

31) Compounds of formula (XXIII) can be prepared by reduction of compounds of formula (XXXVII) with a metal hydride, for instance according to a method developed in the literature (ARKIVOC, 2003, 5, And U.S. Pat. No. 4,524,206).

32) Compounds of formula (XXXVII) can be prepared by reaction of compound of formula (XXXVI) with a compound of formula (XXI) as described in 15).

33) Compounds of formula (XXXVII) can be prepared by reaction of compound of formula (XXXVIII) with a compound of formula (XXVII) under standard substitution reaction conditions.

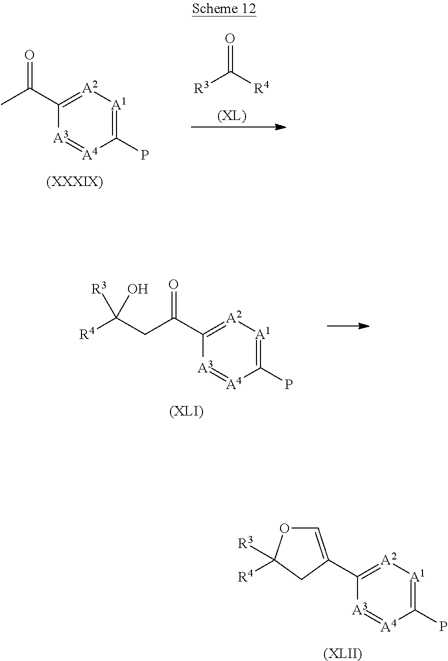

34) Compounds of formula (XLII) can be prepared by reacting a compound of formula (XLI) with trimethylsilyldiazomethane, in the presence of an organometallic reagent, such as methyl lithium, in a suitable solvent, such as tetrahydrofuran, diethyl ether, N, N-dimethylformamide or dimethoxyethane. The reaction is carried out at a temperature of from −78° C. to 100° C., preferably from −78° C. to ambient temperature. Compounds of formula (XLII) are either known compounds or can be prepared using methods described for example in WO2007074789, preferably by reacting a compound of formula (XXXIX) with a ketone of formula (XL).

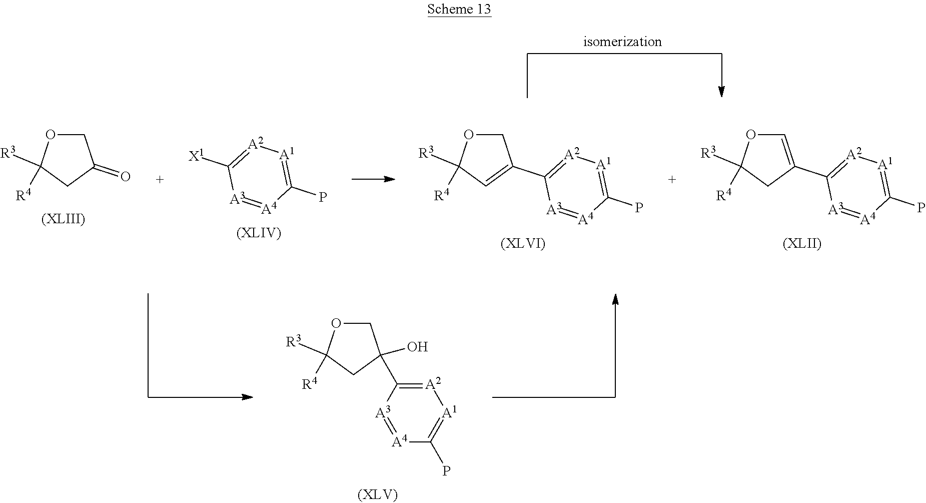

35) Alternatively, 2,3-dihydrofuran compounds of formula (XLII) may be prepared by isomerisation of 2,5-dihydrofuran of formula (XLVI) using a metal catalyst such as RhCl(PPh₃)₃, RhH(PPh₃)₄, H₂Ru(CO)(PPh₃)₃, RuCl₃, HClRu(CO)(PPh₃)₃ or H₂Ru(PPh₃)₄ in a solvent such as toluene or an alcoholic solvent such as ethanol at a temperature of between room temperature and 150° C., preferably between 80° C. and 120° C. Such conditions of isomerisation of 2,5-dihydrofuran compounds have been described in Chem. Eur. J. 2003, 9, 4442-4451 using the general catalytic isomerisation described by M. Mori et al in J. Org. Chem. 2000, 65, 3966-3970 or M. Bartok et al in J. Organomet. Chem. 1985, 297, C₃₇-C₄₀. Alternatively, the isomerisation may be performed in the presence of basic oxide metal catalysts such as MgO, CaO, SrO, or La₂O₃ as described by K. Tanabe in Chem. Lett. 1981, 341-342 for the isomerisation of 2,5-dihydrofuran.

36) Compounds of formula (XLII) and (XLVI) can be prepared by reacting a compound of formula (XLIV) wherein X¹ is a leaving group, for example a halogen, such as iodo or bromo with a compound of formula (XLIII), in the presence of a metal, such as catalyst, such as magnesium, lithium, indium, cerium or zinc, in a suitable solvent, such as tetrahydrofuran, diethyl ether or N, N-dimethylformamide. The reaction is carried out at a temperature of from −78° C. to 100° C., preferably from −78° C. to ambient temperature.

37) Compounds of formula (XLV) can be prepared by reacting a compound of formula (XLIII) with a compound of formula (XLIV), in the presence of a metal, such as magnesium, indium, cerium, zinc, or an organolithium reagent, such as n-butyl lithium, in a suitable solvent, such as tetrahydrofuran, diethyl ether or N, N-dimethylformamide. The reaction is carried out at a temperature of from −100° C. to 100° C., preferably from −100° C. to ambient temperature.

38) Compounds of formula (XLII) and (XLVI) can be prepared by reacting a compound of formula (XLV) in the presence of an acid, such as p-toluenesulfonic acid or sulphuric acid, or in the presence of a dehydrating agent, such as POCl₃ in a suitable solvent, such as tetrahydrofuran, diethyl ether or dichloromethane. The reaction is carried out at a temperature of from −78° C. to 100° C., preferably from −40° C. to ambient temperature.

39) Alternatively, compounds of formula (XLII) and (XLVI) can be obtained by reacting a compound of formula (XLV) in the presence of a chlorinating agent, such as thionyl chloride or oxalyl chloride, or an acetylating agent, such as acetic anhydride in the presence of a base, such as triethylamine, potassium carbonate or pyridine, in a suitable solvent, such as tetrahydrofuran, diethyl ether or dichloromethane. The reaction is carried out at a temperature of from −78° C. to 100° C., preferably from −40° C. to ambient temperature.

40) Compounds of formula (XLIV) are either known compounds or can be prepared by known methods to the person skilled in the art. Compounds of formula (XLIII) can be prepared as described in Scheme 14.

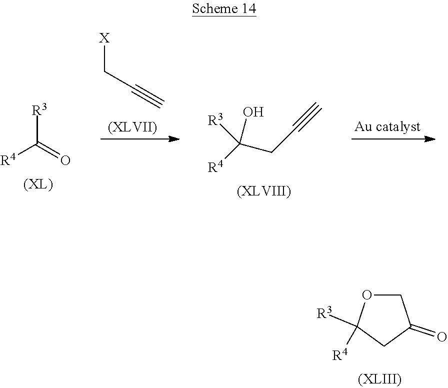

41) Compounds of formula (XLIII) can be prepared by hydrative cyclisation of a compound of formula (XLVIII) These reactions are usually carried out in the presence of a suitable Lewis acid, such as a gold catalyst, as described in J. Am. Chem. Soc., 2010, 132 (10), pp 3258-3259. The reaction is usually carried out using (Triphenylphosphine)gold(I) bis(trifluoromethanesulfonyl)imidate, in the presence of a pyridine N-oxyde, such as 5-Bromo-1-oxy-nicotinic acid methyl ester and an acid, such as methanesulfonic acid, in an aprotic solvent, such as 1,2-dichloroethane. The reaction is carried out at a temperature of from 0° C. to 100° C., preferably from 0° C. to 40° C.

42) Compounds of formula (XLVIII) can be prepared by reacting a ketone of formula (XL) with a compound of formula (XLVII), where X is a halogen. These reactions are usually carried out in the presence of a metal, such as magnesium, lithium, indium, cerium or zinc, in a suitable solvent, such as tetrahydrofuran, diethyl ether or N, N-dimethylformamide. The reaction is carried out at a temperature of from −78° C. to 100° C., preferably from −78° C. to ambient temperature. Alternatively, compounds of formula (XLVIII) can be prepared by reacting a compound of formula (XL) with a compound of formula (XLVII), where X is a trialkylsilyl group. These reactions are usually carried out in the presence of strong base, such as lithium diisopropylamide, in a suitable solvent, such as tetrahydrofuran, diethyl ether or N, N-dimethylformamide. The reaction is carried out at a temperature of from −78° C. to 100° C., preferably from −78° C. to ambient temperature.

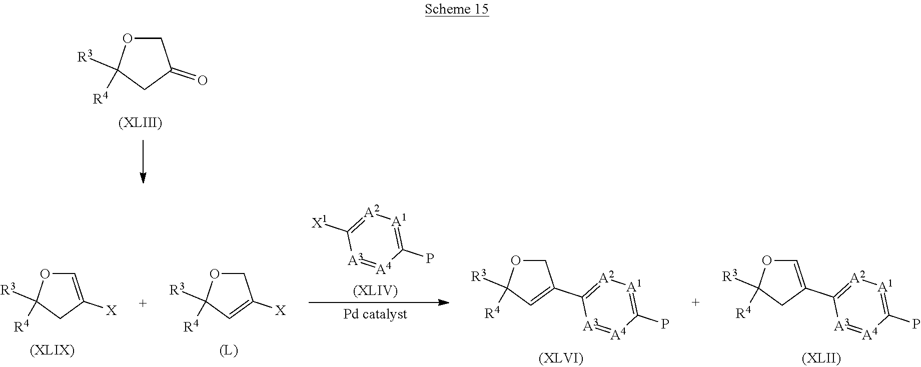

43) Compounds of formula (XLVI) (and compounds of formula (XLII)) can be prepared by reacting a compound of formula (L) (and respectively compounds of formula (XLIX)) wherein X is a leaving group, for example a halogen, such as bromo, or a triflate, with a compound of formula (XLIV) wherein X¹ is a boron derivative, such as a boronic acid, a pinacolboronate, or a trifluoroborate salt, in a Suzuki coupling reaction, in the presence of a palladium catalyst, such as palladium acetate or tetrakis(triphenylphosphine) palladium, in a suitable solvent, such as 1,4-dioxane, toluene, acetonitrile or N, N-dimethylformamide. The reaction is carried out at a temperature of from −20° C. to 150° C., preferably from ambient temperature to 100° C. Alternatively, compounds of formula (XLVI) (and compounds of formula (XLII)) can be prepared by reacting a compound of formula (L) (and respectively compounds of formula (XLIX)) wherein X is a leaving group, for example a halogen, such as bromo, or a triflate with a compound of formula (XLIV) wherein X¹ is a trialkylstannane derivative, such as tributyltin, or respectively an organozinc derivative in a Stille or Negishi coupling reaction, in the presence of a palladium catalyst, such as palladium acetate or tetrakis(triphenylphosphine) palladium, in a suitable solvent, such as 1,4-dioxane, toluene, acetonitrile or N, N-dimethylformamide.

44) Compounds of formula (XLIX) (and compounds of formula (L)) wherein X is a halogen, such as bromo, can be prepared by reacting a compound of formula (XLIII) with a brominating agent, such as phosphoric tribromide, in a suitable solvent, such as tetrahydrofuran, or chloroform, dichloromethane. The reaction is carried out at a temperature of from −40° C. to 100° C., preferably from −40° C. to ambient temperature. Alternatively, compounds of formula (XLIX) (and compounds of formula (L)) wherein X is a triflate, can be prepared by reacting a compound of formula (XLIII) with a triflating agent, such as triflic anhydride or N,N-bis(trifluoromethanesulfonyl)aniline, in the presence of a base, such as 4-picoline, sodium or potassium hexamethyldisilylamide, lithium diisopropylamide, triethylamine or 2,6-lutidine in a suitable solvent, such as tetrahydrofuran, chloroform or dichloromethane. The reaction is carried out at a temperature of from −100° C. to 150° C., preferably from −40° C. to 100° C.

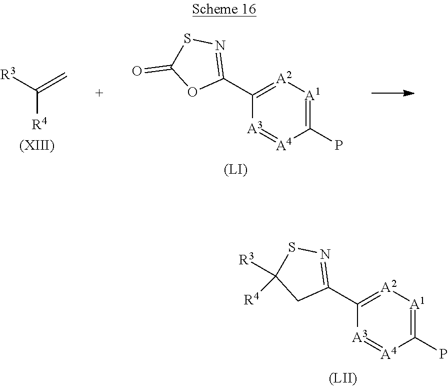

45) Compounds of formula (LII) can be prepared by reacting a compound of formula (LI) with the vinyl compound of formula (XIII) optionally in the presence of a suitable solvent, for example N, N-dimethylformamide, xylene, toluene, chlorobenzene or dichlorobenzene. The reaction can be performed under microwave heating preferably at temperatures up to 200° C. and preferably under neat conditions using a large excess of the compound of formula (XIII) (e.g. 40 equivalents). Vinyl compounds of formula (XIII) are commercially available or can be made by methods known to a person skilled in the art. Compounds of formula (LI) can be made by methods known to a person skilled in the art, as described in journal of Organic Chemistry (1981), 46(4), 771.

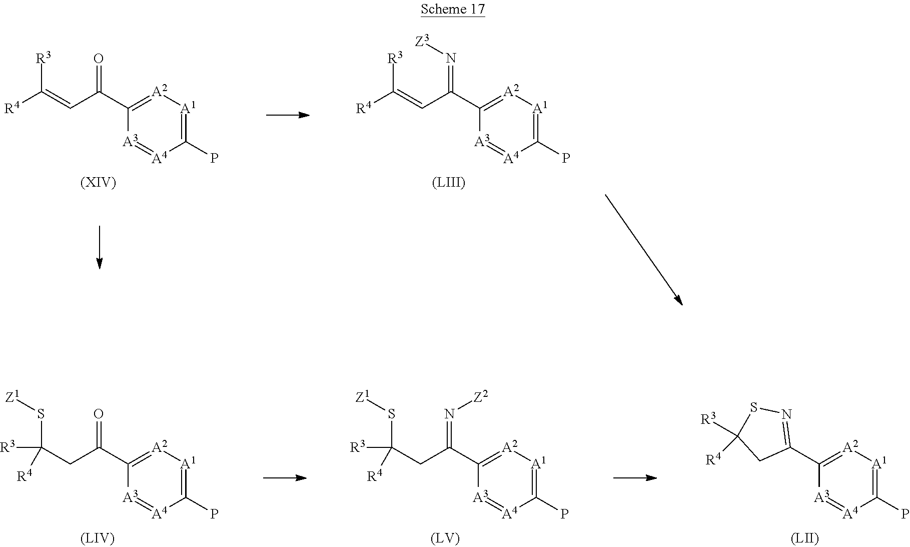

46) Compounds of formula (LIV) wherein Z¹ is hydrogen or cyano or halogen or C₁-C₈alkyl or aryl-C₁-C₄alkylene- or C₁-C₈alkylcarbonyl- or arylsulfonyl- or arylthio-, can be obtained by reacting an unsaturated ketone of formula (XIV), with a sulfur nucleophile, such as thioacetic acid, hydrogen sulfide, sodium sulfide, ammonium sulfide, thiourea, benzylmercaptan, Sodium benzenethiosulfonate, potassium thiocyanate, sodium thiocyanate, sodium thiomethoxide or tert-butyl mercaptan as shown on Scheme 17. Such reactions can be performed optionally in the presence of a base, such as sodium hydroxide, sodium ethoxide, sodium methoxide, sodium tert-butoxide or potassium hydroxide. Sometimes, such reactions can also be performed in the presence of an acid, for example p-toluenesulfonic acid, hydrochloric acid, acetic acid, in a solvent, such as methanol, ethanol, N,N-dimethylformamide, toluene, dichloromethane, ethyl acetate, acetonitrile or chlorobenzene or water, or mixtures thereof, at a temperature of from 0° C. to 100° C., preferably from ambient temperature to 80° C. Such conditions are described, for example, in Journal of the American Chemical Society (1949), 71, 3554-5 or in Tetrahedron: Asymmetry (2003), 14(1), 113-117 and Journal of Organic Chemistry (1996), 61, 1986.

47) Compounds of formula (LIII) wherein Z³ is thiol or aryl substituted C₁-C₈alkylsulfinyl-, can be made by reaction of the ketone of formula (XIV) with an amine, such as triphenylmethanesulfenamide. Such reactions are usually carried out in the presence of an acid or not, for example p-toluenesulfonic acid, hydrochloric acid, acetic acid, optionally in the presence of a solvent, for example an alcohol, such as methanol or ethanol, or toluene, dichloromethane, water, or mixtures thereof. The reaction can be carried out in the presence or the absence of a dehydrating agent, such as anhydrous magnesium sulfate or molecular sieves. It can also be performed using a Dean Stark or Soxhlet apparatus that enables a constant removal of the water formed during the reaction. The reaction is carried out at a temperature of from 0° C. to 100° C., preferably from 15° C. to 30° C., in particular at ambient temperature.

48) Compounds of formula (LV) wherein Z² is hydrogen or hydroxyl or C₁-C₈alkoxy- or C1-C₈alkylsulfonyloxy- or C₁-C₈arylsulfonyloxy- or aryl-C₁-C₄alkylene- or aryl, can be made by reaction of the ketone of formula (LIV) with an amine, such as hydroxylamine hydrochloride, methoxylamine or ammonia. Such reactions are carried out in the presence of a base, for example an organic base, such as triethylamine or sodium acetate, or an inorganic base, such as sodium hydrogen carbonate, optionally in the presence of a solvent, for example an alcohol, such as methanol or ethanol, or water, or mixtures thereof. Such reactions can also be carried out in the presence of an acid or not, for example p-toluenesulfonic acid, hydrochloric acid, acetic acid, optionally in the presence of a solvent, for example an alcohol, such as methanol or ethanol, or toluene, dichloromethane, water, or mixtures thereof. The reaction can be carried out in the presence or the absence of a dehydrating agent, such as anhydrous magnesium sulfate or molecular sieves. It can also be performed using a Dean Stark or Soxhlet apparatus that enables a constant removal of the water formed during the reaction. The reaction is carried out at a temperature of from 0° C. to 100° C., preferably from 15° C. to 30° C., in particular at ambient temperature.

49) Compounds of formula (LII) can be obtained by cyclizing a compound of formula (LIII) wherein Z³ is thiol. Such reactions are usually carried out in the presence of an acid or not, for example p-toluenesulfonic acid, optionally in the presence of a solvent, for example dichloroethane, methanol, ethanol, toluene, dichloromethane or chlorobenzene. The reaction is carried out at a temperature of from 0° C. to 200° C., preferably from 25° C. to 100° C. Alternatively, compounds of formula (LII) can be obtained by cyclizing a compound of formula (LIII) wherein Z³ is aryl substituted C₁-C₈alkylsulfinyl-. Such reactions are usually carried out in the presence of an acid or not, for example p-toluenesulfonic acid, trifluoroacetic acid or hydrochloric acid, optionally in the presence of a solvent, for example dichloroethane, methanol, ethanol, toluene, dichloromethane or chlorobenzene. The reaction is carried out at a temperature of from 0° C. to 200° C., preferably from 25° C. to 100° C. Such reactions usually involve first the deprotection of the thiol to give a compound of formula (X) wherein Z³ is thiol, followed by the cyclization.

50) Compounds of formula (LII) can be obtained from compound of formula (LV) wherein Z¹ is hydrogen or cyano or halogen or C₁-C₈alkyl or aryl-C₁-C₄alkylene- or C₁-C₈alkylcarbonyl- or arylsulfonyl- or arylthio-, and Z² is hydrogen or hydroxyl or C₁-C₈alkoxy- or C₁-C₈alkylsulfonyloxy- or C₁-C₈arylsulfonyloxy- or aryl-C₁-C₄alkylene- or aryl. Such reactions usually involve the deprotection of Z¹ and of Z² or of both groups. The reaction can then involve the following intermediates:

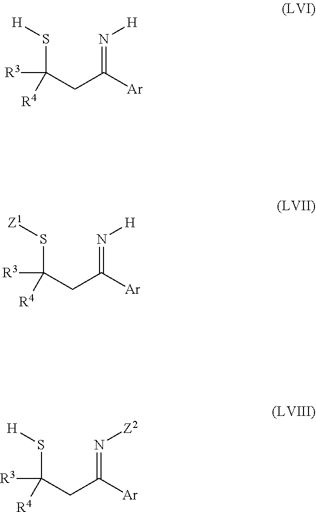

Depending on the nature of Z¹ and Z², the deprotection conditions are different and can be made by methods known to a person skilled in the art or as described in T. W. Green, P. G. M. Wuts, Protective Groups in Organic Synthesis, Wiley-Interscience, New York, 1999, 564-566, 740-743.

51) Compounds of formula (LII) can be obtained from compound of formula (LVI) from an oxidation step. Such reactions are usually carried out in the presence of an oxidant, for example iodine, bromine, thionyl chloride, Bis(trifluoroacetoxy)iodobenzene; The reaction can be carried out in the presence of an acid or not, such as trifluoroacetic acid or acetic acid, optionally in the presence of a solvent, for example dichloroethane, dimethylsulfoxide, N,N-dimethylformamide, methanol, ethanol, toluene, dichloromethane, ethyl acetate or chlorobenzene. The reaction is carried out at a temperature of from 0° C. to 200° C., preferably from 25° C. to 100° C. Such transformations, including reaction conditions and suitable catalyst, are described in Journal of the Chemical Society, Perkin Transactions 1: Organic and Bio-Organic Chemistry (1972-1999) (1985), (1), 153-7 and Organic Letters (2006), 8(21), 4811-4813. Similarly, compounds of formula (LII) can be obtained from a compound of formula (LVII) wherein Z¹ is arylsulfonyl- or arylthio-, by an oxidation step, are described in Journal of Organic Chemistry (1990), 55(13), 4156-62.

52) Compounds of formula (LII) can be obtained from compound of formula (LVIII) wherein Z² is C₁-C₈alkoxy-. Such reactions are usually carried out in the presence of a copper (I) reagent, such copper-3-methylsalicylate. The reaction can be carried out in the presence of a solvent, for example dichloroethane, dimethylsulfoxide, N,N-dimethylformamide, methanol, ethanol, toluene, dichloromethane, ethyl acetate or chlorobenzene. The reaction is carried out at a temperature of from 0° C. to 200° C., preferably from 25° C. to 100° C., or under microwave heating conditions. Such transformations are described in Journal of the American Chemical Society (2011), 133, 6403-6410.

53) Alternatively, compounds of formula (LII) can be obtained directly from a compound of formula (LVII) wherein Z¹ is hydrogen. Such reactions are usually carried out in the presence chloramines, formed in situ from ammonia and chlorine or sodium hypochlorite or hypochlorous acid, optionally in the presence of a solvent, for example dichloroethane, methanol, ethanol, toluene, dichloromethane or chlorobenzene. The reaction is carried out at a temperature of from −80° C. to 40° C., preferably below −40° C.

54) Alternatively, compounds of formula (LII) can be obtained directly from a compound of formula (LIV) wherein Z¹ is cyano or halogen or arylsulfonyl- or arylthio-. Such reactions are usually carried out in the presence ammonia, optionally in the presence of a solvent, for example dichloroethane, tetrahydrofuran, methanol, ethanol, toluene, dichloromethane or chlorobenzene. The reaction is carried out at a temperature of from −80° C. to 80° C.

55) Alternatively, compounds of formula (LII) can be obtained directly from a compound of formula (LIV) wherein Z¹ is aryl-C₁-C₄alkylene. Such reactions are usually carried out in two steps—The first one involves the treatment of a compound of formula (LIV) wherein Z¹ is aryl-C₁-C₄alkylene by a suitable oxidant, such as sulfuryl chloride or chlorine, in the presence of a solvent, for example dichloroethane, methanol, ethanol, toluene, dichloromethane or chlorobenzene, to provide a compound of formula (LIV) wherein Z¹ is chlorine. The second step then involves the treatment a compound of formula (LIV) wherein Z¹ is chlorine by an ammonia source, such as ammonia or ammonium bromide in the presence of a base, in the presence of a solvent, for example dichloroethane, methanol, ethanol, toluene, tetrahydrofuran, dichloromethane or chlorobenzene. Both steps are usually carried out at a temperature of from −80° C. to 80° C.

56) Alternatively, compounds of formula (LII) can be obtained directly from a compound of formula (LIV) wherein Z¹ is hydrogen. Such reactions are usually carried out in the presence of a suitable nitrogen electrophile, such as Hydroxylamine-O-sulfonic acid. Such reactions are carried out in the presence of a base, for example an organic base, such as triethylamine or sodium acetate, or an inorganic base, such as sodium hydrogen carbonate, sodium hydroxide or potassium hydroxide optionally in the presence of a solvent, for example tetrahydrofuran, toluene, an alcohol, such as methanol or ethanol, or water, or mixtures thereof. The reaction is carried out at a temperature of from −80° C. to 80° C.

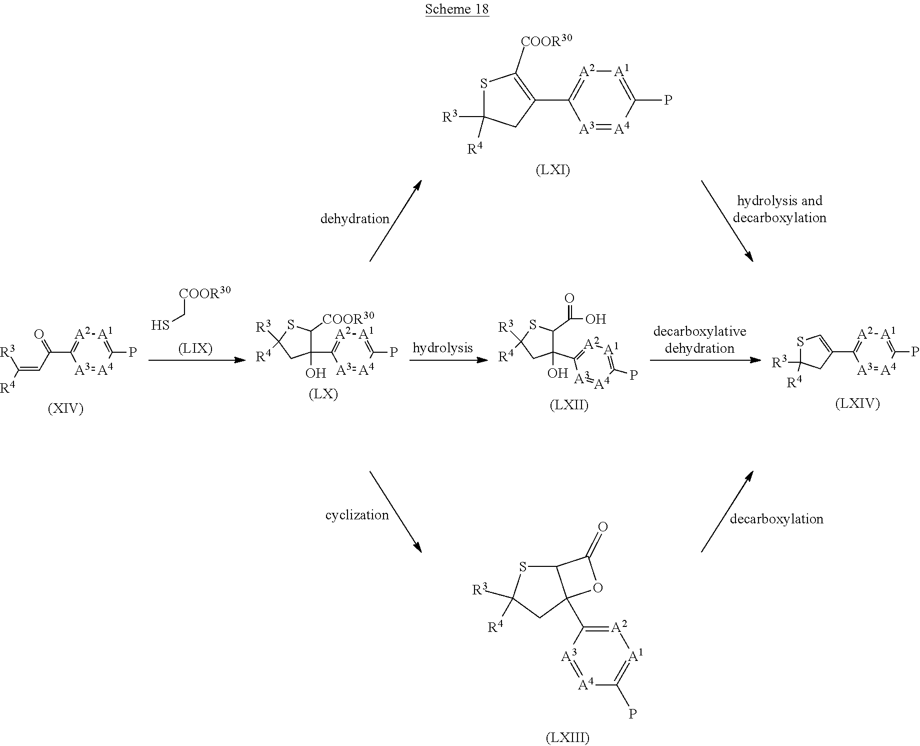

56) Compounds of formula (LXIV) can be obtained from compounds of formula (LXI), wherein R³⁰ is C₁-C₁₅alkyl, by hydrolysis using a suitable base such as MOH, M₂CO₃, MHCO₃ wherein M is an alkali metal such as lithium, sodium, potassium, cesium, barium etc. or an alkoxide, or in the presence of acid, such as trifluoroacetic acid or hydrochloric acid, followed by decarboxylation by heating with or without a base.

The reaction is carried out at a temperature of from −20° C. to 200° C., in the presence or the absence of a suitable solvent.

57) Compounds of formula (LXIV) can be obtained from compounds of formula (LXIII) by subjecting then to a decarboxylative dehydration following the procedures known in literature.

58) Compounds of formula (LXIV) can also be obtained from compounds of formula (LX) via a two-step sequence: first the compounds of formula (LX) can be converted to the bicyclic intermediates of formula (LXIII) using with various activating agents like protic acids HCl, H₂SO₄, H₃PO₄ etc or Lewis acid such BF₃.OEt₂, or using dehydrating agents, such as MsCl, Tf₂O, SOCl₂, (COCl)₂ and POCl₃. Then, the compounds of formula (LXIII) can be transformed into the compounds of formula (LXIV) by heating to a temperature of from 30° C. to 150° C., in the presence or the absence of a suitable solvent.

59) Compounds of formula (LXI) can be obtained from compounds of formula (LX) by subjecting then to a dehydration following the procedures known in literature, using various activating agents like protic acids HCl, H₂SO₄, H₃PO₄ etc. or Lewis acid such BF₃.OEt₂, or using dehydrating agents, such as MsCl, Tf₂O, SOCl₂, (COCl)₂ and POCl₃.

60) Compounds of formula (LX) can be obtained from compounds of formula (XIV) and compounds of formula (LIX) in presence or absence of bases such as MOH, M₂CO₃, MHCO₃ wherein M is an alkali metal such as lithium, sodium, potassium, cesium, barium etc. or an alkoxide, at a temperature of from −20° C. to 200° C.

A compound of formula (I) can be converted in a manner known per se into another compound of formula (I) by replacing one or more substituents of the starting compound of formula (I) in the customary manner by (an)other substituent(s) according to the invention.

Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

Salts of compounds of formula (I) can be prepared in a manner known per se. Thus, for example, acid addition salts of compounds of formula (I) are obtained by treatment with a suitable acid or a suitable ion exchanger reagent and salts with bases are obtained by treatment with a suitable base or with a suitable ion exchanger reagent.

Salts of compounds of formula (I) can be converted in the customary manner into the free compounds I, acid addition salts, for example, by treatment with a suitable basic compound or with a suitable ion exchanger reagent and salts with bases, for example, by treatment with a suitable acid or with a suitable ion exchanger reagent.

Salts of compounds of formula (I) can be converted in a manner known per se into other salts of compounds of formula (I), acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture. Depending on the procedure or the reaction conditions, the compounds of formula (I), which have salt-forming properties can be obtained in free form or in the form of salts.

The compounds of formula (I) and, where appropriate, the tautomers thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule; the invention relates to the pure isomers and also to all isomer mixtures which are possible and is to be understood in each case in this sense hereinabove and hereinbelow, even when stereochemical details are not mentioned specifically in each case.

Diastereomer mixtures or racemate mixtures of compounds of formula (I), in free form or in salt form, which can be obtained depending on which starting materials and procedures have been chosen can be separated in a known manner into the pure diasteromers or racemates on the basis of the physicochemical differences of the components, for example by fractional crystallization, distillation and/or chromatography.

Enantiomer mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.

Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

N-oxides can be prepared by reacting a compound of the formula (I) with a suitable oxidizing agent, for example the H₂O₂/urea adduct in the presence of an acid anhydride, e.g. trifluoroacetic anhydride. Such oxidations are known from the literature, for example from J. Med. Chem., 32 (12), 2561-73, 1989 or WO200015615.

It is advantageous to isolate or synthesize in each case the biologically more effective isomer, for example enantiomer or diastereomer, or isomer mixture, for example enantiomer mixture or diastereomer mixture, if the individual components have a different biological activity.

The compounds of formula (I) and, where appropriate, the tautomers thereof, in each case in free form or in salt form, can, if appropriate, also be obtained in the form of hydrates and/or include other solvents, for example those which may have been used for the crystallization of compounds which are present in solid form.

The compounds of formula (I) according to the invention are preventively and/or curatively valuable active ingredients in the field of pest control, even at low rates of application, which have a very favourable biocidal spectrum and are well tolerated by warm-blooded species, fish and plants. The pests which may be combated and controlled by the use of the compounds of the invention include those pests associated with agriculture (which term includes the growing of crops for food and fiber products), horticulture and animal husbandry, companion animals, forestry and the storage of products of vegetable origin (such as fruit, grain and timber); those pests associated with the damage of man-made structures and the transmission of diseases of man and animals; and also nuisance pests (such as flies). The active ingredients according to the invention act against all or individual developmental stages of normally sensitive, but also resistant, animal pests, such as insects or representatives of the order Acarina. The insecticidal or acaricidal activity of the active ingredients according to the invention can manifest itself directly, i. e. in destruction of the pests, which takes place either immediately or only after some time has elapsed, for example during ecdysis, or indirectly, for example in a reduced oviposition and/or hatching rate, a good activity corresponding to a destruction rate (mortality) of at least 50 to 60%.

Examples of the above mentioned animal pests are:

from the order Acarina, for example,
Acalitus spp, Aculus spp, Acaricalus spp, Aceria spp, Acarus siro, Amblyomma spp., Argas spp., Boophilus spp., Brevipalpus spp., Bryobia spp, Calipitrimerus spp., Chorioptes spp., Dermanyssus gallinae, Dermatophagoides spp, Eotetranychus spp, Eriophyes spp., Hemitarsonemus spp, Hyalomma spp., Ixodes spp., Olygonychus spp, Ornithodoros spp., Polyphagotarsone latus, Panonychus spp., Phyllocoptruta oleivora, Phytonemus spp, Polyphagotarsonemus spp, Psoroptes spp., Rhipicephalus spp., Rhizoglyphus spp., Sarcoptes spp., Steneotarsonemus spp, Tarsonemus spp. and Tetranychus spp.;
from the order Anoplura, for example,

### Haematopinus spp., Linognathus spp., Pediculus spp., Pemphigus spp. and Phylloxera spp.;

from the order Coleoptera, for example,
Agriotes spp., Amphimallon majale, Anomala orientalis, Anthonomus spp., Aphodius spp, Astylus atromaculatus, Ataenius spp, Atomaria linearis, Chaetocnema tibialis, Cerotoma spp, Conoderus spp, Cosmopolites spp., Cotinis nitida, Curculio spp., Cyclocephala spp, Dermestes spp., Diabrotica spp., Diloboderus abderus, Epilachna spp., Eremnus spp., Heteronychus arator, Hypothenemus hampei, Lagria vilosa, Leptinotarsa decemLineata, Lissorhoptrus spp., Liogenys spp, Maecolaspis spp, Maladera castanea, Megascelis spp, Melighetes aeneus, Melolontha spp., Myochrous armatus, Orycaephilus spp., Otiorhynchus spp., Phyllophaga spp, Phlyctinus spp., Popillia spp., Psylliodes spp., Rhyssomatus aubtilis, Rhizopertha spp., Scarabeidae, Sitophilus spp., Sitotroga spp., Somaticus spp, Sphenophorus spp, Sternechus subsignatus, Tenebrio spp., Tribolium spp. and Trogoderma spp.;
from the order Diptera, for example,
Aedes spp., Anopheles spp, Antherigona soccata, Bactrocea oleae, Bibio hortulanus, Bradysia spp, Calliphora erythrocephala, Ceratitis spp., Chrysomyia spp., Culex spp., Cuterebra spp., Dacus spp., Delia spp, Drosophila melanogaster, Fannia spp., Gastrophilus spp., Geomyza tripunctata, Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis spp, Rivelia quadrifasciata, Scatella spp, Sciara spp., Stomoxys spp., Tabanus spp., Tannia spp. and Tipula spp.;
from the order Hemiptera, for example,
Acanthocoris scabrator, Acrosternum spp, Adelphocoris lineolatus, Amblypelta nitida, Bathycoelia thalassina, Blissus spp, Cimex spp., Clavigralla tomentosicollis, Creontiades spp, Distantiella theobroma, Dichelops furcatus, Dysdercus spp., Edessa spp, Euchistus spp., Eurydema pulchrum, Eurygaster spp., Halyomorpha halys, Horcias nobilellus, Leptocorisa spp., Lygus spp, Margarodes spp, Murgantia histrionic, Neomegalotomus spp, Nesidiocoris tenuis, Nezara spp., Nysius simulans, Oebalus insularis, Piesma spp., Piezodorus spp, Rhodnius spp., Sahlbergella singularis, Scaptocoris castanea, Scotinophara spp. , Thyanta spp, Triatoma spp., Vatiga illudens; Acyrthosium pisum, Adalges spp, Agalliana ensigera, Agonoscena targionii, Aleurodicus spp, Aleurocanthus spp, Aleurolobus barodensis, Aleurothrixus floccosus, Aleyrodes brassicae, Amarasca biguttula, Amritodus atkinsoni, Aonidiella spp., Aphididae, Aphis spp., Aspidiotus spp., Aulacorthum solani, Bactericera cockerelli, Bemisia spp, Brachycaudus spp, Brevicoryne brassicae, Cacopsylla spp, Cavariella aegopodii Scop., Ceroplaster spp., Chrysomphalus aonidium, Chrysomphalus dictyospermi, Cicadella spp, Cofana spectra, Cryptomyzus spp, Cicadulina spp, Coccus hesperidum, Dalbulus maidis, Dialeurodes spp, Diaphorina citri, Diuraphis noxia, Dysaphis spp, Empoasca spp., Eriosoma larigerum, Erythroneura spp., Gascardia spp., Glycaspis brimblecombei, Hyadaphis pseudobrassicae, Hyalopterus spp, Hyperomyzus pallidus, Idioscopus clypealis, Jacobiasca lybica, Laodelphax spp., Lecanium corni, Lepidosaphes spp., Lopaphis erysimi, Lyogenys maidis, Macrosiphum spp., Mahanarva spp, Metcalfa pruinosa, Metopolophium dirhodum, Myndus crudus, Myzus spp., Neotoxoptera sp, Nephotettix spp., Nilaparvata spp., Nippolachnus piri Mats, Odonaspis ruthae, Oregma lanigera Zehnter, Parabemisia myricae, Paratrioza cockerelli, Parlatoria spp., Pemphigus spp., Peregrinus maidis, Perkinsiella spp, Phorodon humuli, Phylloxera spp, Planococcus spp., Pseudaulacaspis spp., Pseudococcus spp., Pseudatomoscelis seriatus, Psylla spp., Pulvinaria aethiopica, Quadraspidiotus spp., Quesada gigas, Recilia dorsalis, Rhopalosiphum spp., Saissetia spp., Scaphoideus spp., Schizaphis spp., Sitobion spp., Sogatella furcifera, Spissistilus festinus, Tarophagus Proserpina, Toxoptera spp, Trialeurodes spp, Tridiscus sporoboli, Trionymus spp, Trioza erytreae, Unaspis citri, Zygina flammigera, Zyginidia scutellaris;
from the order Hymenoptera, for example,
Acromyrmex, Arge spp, Atta spp., Cephus spp., Diprion spp., Diprionidae, Gilpinia polytoma, Hoplocampa spp., Lasius spp., Monomorium pharaonis, Neodiprion spp., Pogonomyrmex spp, Slenopsis invicta, Solenopsis spp. and Vespa spp.;
from the order Isoptera, for example,

### Coptotermes Spp, Corniternes Cumulans, Incisitermes Spp, Macrotermes Spp, Mastotermes Spp, Microtermes Spp, Reticulitermes Spp.; Solenopsis Geminate

from the order Lepidoptera, for example,
Acleris spp., Adoxophyes spp., Aegeria spp., Agrotis spp., Alabama argillaceae, Amylois spp., Anticarsia gemmatalis, Archips spp., Argyresthia spp, Argyrotaenia spp., Autographa spp., Bucculatrix thurberiella, Busseola fusca, Cadra cautella, Carposina nipponensis, Chilo spp., Choristoneura spp., Chrysoteuchia topiaria, Clysia ambiguella, Cnaphalocrocis spp., Cnephasia spp., Cochylis spp., Coleophora spp., Colias lesbia, Cosmophila flava, Crambus spp, Crocidolomia binotalis, Cryptophlebia leucotreta, Cydalima perspectalis, Cydia spp., Diaphania perspectalis, Diatraea spp., Diparopsis castanea, Earias spp., Eldana saccharina, Ephestia spp., Epinotia spp, Estigmene acrea, Etiella zinckinella, Eucosma spp., Eupoecilia ambiguella, Euproctis spp., Euxoa spp., Feltia jaculiferia, Grapholita spp., Hedya nubiferana, Heliothis spp., Hellula undalis, Herpetogramma spp, Hyphantria cunea, Keiferia lycopersicella, Lasmopalpus lignosellus, Leucoptera scitella, Lithocollethis spp., Lobesia botrana, Loxostege bifidalis, Lymantria spp., Lyonetia spp., Malacosoma spp., Mamestra brassicae, Manduca sexta, Mythimna spp, Noctua spp, Operophtera spp., Orniodes indica, Ostrinia nubilalis, Pammene spp., Pandemis spp., Panolis flammea, Papaipema nebris, Pectinophora gossypiela, Perileucoptera coffeella, Pseudaletia unipuncta, Phthorimaea operculella, Pieris rapae, Pieris spp., Plutella xylostella, Prays spp., Pseudoplusia spp, Rachiplusia nu, Richia albicosta, Scirpophaga spp., Sesamia spp., Sparganothis spp., Spodoptera spp., Sylepta derogate, Synanthedon spp., Thaumetopoea spp., Tortrix spp., Trichoplusia ni, Tuta absoluta, and Yponomeuta spp.;
from the order Mallophaga, for example,

### Damalinea spp. and Trichodectes spp.;

from the order Orthoptera, for example,
Blatta spp., Blattella spp., Gryllotalpa spp., Leucophaea maderae, Locusta spp., Neocurtilla hexadactyla, Periplaneta spp., Scapteriscus spp, and Schistocerca spp.;
from the order Psocoptera, for example,

### Liposcelis spp.;

from the order Siphonaptera, for example,
Ceratophyllus spp., Ctenocephalides spp. and Xenopsylla cheopis;
from the order Thysanoptera, for example,
Calliothrips phaseoli, Frankliniella spp., Heliothrips spp, Hercinothrips spp., Parthenothrips spp, Scirtothrips aurantii, Sericothrips variabilis, Taeniothrips spp., Thrips spp;
from the order Thysanura, for example,
Lepisma saccharina.

The active ingredients according to the invention can be used for controlling, i. e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp or jute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family and latex plants.

The compositions and/or methods of the present invention may be also used on any ornamental and/or vegetable crops, including flowers, shrubs, broad-leaved trees and evergreens.

For example the invention may be used on any of the following ornamental species: Ageratum spp., Alonsoa spp., Anemone spp., Anisodontea capsenisis, Anthemis spp., Antirrhinum spp., Aster spp., Begonia spp. (e.g. B. elatior, B. semperflorens, B. tubéreux), Bougainvillea spp., Brachycome spp., Brassica spp. (ornamental), Calceolaria spp., Capsicum annuum, Catharanthus roseus, Canna spp., Centaurea spp., Chrysanthemum spp., Cineraria spp. (C. maritime), Coreopsis spp., Crassula coccinea, Cuphea ignea, Dahlia spp., Delphinium spp., Dicentra spectabilis, Dorotheantus spp., Eustoma grandiflorum, Forsythia spp., Fuchsia spp., Geranium gnaphalium, Gerbera spp., Gomphrena globosa, Heliotropium spp., Helianthus spp., Hibiscus spp., Hortensia spp., Hydrangea spp., Hypoestes phyllostachya, Impatiens spp. (I. Walleriana), Iresines spp., Kalanchoe spp., Lantana camara, Lavatera trimestris, Leonotis leonurus, Lilium spp., Mesembryanthemum spp., Mimulus spp., Monarda spp., Nemesia spp., Tagetes spp., Dianthus spp. (carnation), Canna spp., Oxalis spp., Bellis spp., Pelargonium spp. (P. peltatum, P. Zonale), Viola spp. (pansy), Petunia spp., Phlox spp., Plecthranthus spp., Poinsettia spp., Parthenocissus spp. (P. quinquefolia, P. tricuspidata), Primula spp., Ranunculus spp., Rhododendron spp., Rosa spp. (rose), Rudbeckia spp., Saintpaulia spp., Salvia spp., Scaevola aemola, Schizanthus wisetonensis, Sedum spp., Solanum spp., Surfinia spp., Tagetes spp., Nicotinia spp., Verbena spp., Zinnia spp. and other bedding plants.

For example the invention may be used on any of the following vegetable species: Allium spp. (A. sativum, A. cepa, A. oschaninii, A. Porrum, A. ascalonicum, A. fistulosum), Anthriscus cerefolium, Apium graveolus, Asparagus officinalis, Beta vulgarus, Brassica spp. (B. Oleracea, B. Pekinensis, B. rapa), Capsicum annuum, Cicer arietinum, Cichorium endivia, Cichorum spp. (C. intybus, C. endivia), Citrillus lanatus, Cucumis spp. (C. sativus, C. melo), Cucurbita spp. (C. pepo, C. maxima), Cyanara spp. (C. scolymus, C. cardunculus), Daucus carota, Foeniculum vulgare, Hypericum spp., Lactuca sativa, Lycopersicon spp. (L. esculentum, L. lycopersicum), Mentha spp., Ocimum basilicum, Petroselinum crispum, Phaseolus spp. (P. vulgaris, P. coccineus), Pisum sativum, Raphanus sativus, Rheum rhaponticum, Rosemarinus spp., Salvia spp., Scorzonera hispanica, Solanum melongena, Spinacea oleracea, Valerianella spp. (V. locusta, V. eriocarpa) and Vicia faba.

Preferred ornamental species include African violet, Begonia, Dahlia, Gerbera, Hydrangea, Verbena, Rosa, Kalanchoe, Poinsettia, Aster, Centaurea, Coreopsis, Delphinium, Monarda, Phlox, Rudbeckia, Sedum, Petunia, Viola, Impatiens, Geranium, Chrysanthemum, Ranunculus, Fuchsia, Salvia, Hortensia, rosemary, sage, St. Johnswort, mint, sweet pepper, tomato and cucumber.

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatos) and Chilo supressalis (preferably in rice).

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatos) and Chilo supressalis (preferably in rice).

In a further aspect, the invention may also relate to a method of controlling damage to plant and parts thereof by plant parasitic nematodes (Endoparasitic-, Semiendoparasitic- and Ectoparasitic nematodes), especially plant parasitic nematodes such as root knot nematodes, Meloidogyne hapla, Meloidogyne incognita, Meloidogyne javanica, Meloidogyne arenaria and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Pratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species, such as Subanguina spp., Hypsoperine spp., Macroposthonia spp., Melinius spp., Punctodera spp., and Quinisulcius spp.

The compounds of the invention may also have activity against the molluscs. Examples of which include, for example, Ampullariidae; Arion (A. ater, A. circumscriptus, A. hortensis, A. rufus); Bradybaenidae (Bradybaena fruticum); Cepaea (C. hortensis, C. Nemoralis); ochlodina; Deroceras (D. agrestis, D. empiricorum, D. laeve, D. reticulatum); Discus (D. rotundatus); Euomphalia; Galba (G. trunculata); Helicelia (H. itala, H. obvia); Helicidae Helicigona arbustorum); Helicodiscus; Helix (H. aperta); Limax (L. cinereoniger, L. flavus, L. marginatus, L. maximus, L. tenellus); Lymnaea; Milax (M. gagates, M. marginatus, M. sowerbyi); Opeas; Pomacea (P. canaticulata); Vallonia and Zanitoides.

Crops are to be understood as also including those crops which have been rendered tolerant to herbicides like bromoxynil or classes of herbicides such as ALS-, EPSPS-, GS-, HPPD- and PPO-inhibitors. An example of a crop that has been rendered tolerant to imidazolinones, e.g. imazamox, by conventional methods of breeding is Clearfield® summer canola. Examples of crops that have been rendered tolerant to herbicides by genetic engineering methods include e.g. glyphosate- and glufosinate-resistant maize varieties commercially available under the trade names RoundupReady®, Herculex I® and LibertyLink®.

Crops are also to be understood as being those which naturally are or have been rendered resistant to harmful insects. This includes plants transformed by the use of recombinant DNA techniques, for example, to be capable of synthesising one or more selectively acting toxins, such as are known, for example, from toxin-producing bacteria. Examples of toxins which can be expressed include δ-endotoxins, vegetative insecticidal proteins (Vip), insecticidal proteins of bacteria colonising nematodes, and toxins produced by scorpions, arachnids, wasps and fungi.

An example of a crop that has been modified to express the Bacillus thuringiensis toxin is the Bt maize KnockOut® (Syngenta Seeds). An example of a crop comprising more than one gene that codes for insecticidal resistance and thus expresses more than one toxin is VipCot® (Syngenta Seeds). Crops or seed material thereof can also be resistant to multiple types of pests (so-called stacked transgenic events when created by genetic modification). For example, a plant can have the ability to express an insecticidal protein while at the same time being herbicide tolerant, for example Herculex I® (Dow AgroSciences, Pioneer Hi-Bred International).

Further areas of use of the compositions according to the invention are the protection of stored goods and store rooms and the protection of raw materials, such as wood, textiles, floor coverings or buildings, and also in the hygiene sector, especially the protection of humans, domestic animals and productive livestock against pests of the mentioned type.

The present invention also provides a method for controlling pests (such as mosquitoes and other disease vectors; see also http://www.who.int/malaria/vector_control/irs/en/). In one embodiment, the method for controlling pests comprises applying the compositions of the invention to the target pests, to their locus or to a surface or substrate by brushing, rolling, spraying, spreading or dipping. By way of example, an IRS (indoor residual spraying) application of a surface such as a wall, ceiling or floor surface is contemplated by the method of the invention. In another embodiment, it is contemplated to apply such compositions to a substrate such as non-woven or a fabric material in the form of (or which can be used in the manufacture of) netting, clothing, bedding, curtains and tents.

In one embodiment, the method for controlling such pests comprises applying a pesticidally effective amount of the compositions of the invention to the target pests, to their locus, or to a surface or substrate so as to provide effective residual pesticidal activity on the surface or substrate. Such application may be made by brushing, rolling, spraying, spreading or dipping the pesticidal composition of the invention. By way of example, an IRS application of a surface such as a wall, ceiling or floor surface is contemplated by the method of the invention so as to provide effective residual pesticidal activity on the surface. In another embodiment, it is contemplated to apply such compositions for residual control of pests on a substrate such as a fabric material in the form of (or which can be used in the manufacture of) netting, clothing, bedding, curtains and tents.

Substrates including non-woven, fabrics or netting to be treated may be made of natural fibres such as cotton, raffia, jute, flax, sisal, hessian, or wool, or synthetic fibres such as polyamide, polyester, polypropylene, polyacrylonitrile or the like. The polyesters are particularly suitable. The methods of textile treatment are known, e.g. WO2008151984, WO2003034823, U.S. Pat. No. 5,631,072, WO2005064072, WO2006128870, EP1724392, WO2005113886 or WO2007090739.

Further areas of use of the compositions according to the invention are the field of tree injection/trunk treatment for all ornamental trees as well all sort of fruit and nut trees.

In the field of tree injection/trunk treatment, the compounds according to the present invention are especially suitable against wood-boring insects from the order Lepidoptera as mentioned above and from the order Coleoptera, especially against woodborers listed in the following tables X and Y:

TABLE X
Examples of exotic woodborers of economic importance.

| Family | Species | Host or Crop Infested |
|---|---|---|
| Buprestidae | Agrilus planipennis | Ash |
| Ceram bycidae | Anoplura glabripennis | Hardwoods |
| Scolytidae | Xylosandrus crassiusculus | Hardwoods |
| | X. mutilatus | Hardwoods |
| | Tomicus piniperda | Conifers |

TABLE Y
Examples of native woodborers of economic importance.

| Family | Species | Host or Crop Infested |
|---|---|---|
| Buprestidae | Agrilus anxius | Birch |
| | Agrilus politus | Willow, Maple |
| | Agrilus sayi | Bayberry, Sweetfern |
| | Agrilus vittaticoillis | Apple, Pear, Cranberry, |
| | | Serviceberry, Hawthorn |
| | Chrysobothris femorata | Apple, Apricot, Beech, Boxelder, |
| | | Cherry, Chestnut, Currant, Elm, |
| | | Hawthorn, Hackberry, Hickory, |
| | | Horsechestnut, Linden, Maple, |
| | | Mountain-ash, Oak, Pecan, Pear, |
| | | Peach, Persimmon, Plum, Poplar, |
| | | Quince, Redbud, Serviceberry, |
| | | Sycamore, Walnut, Willow |
| | Texania campestris | Basswood, Beech, Maple, Oak, |
| | | Sycamore, Willow, Yellow-poplar |
| Cerambycidae | Goes pulverulentus | Beech, Elm, Nuttall, Willow, Black |
| | | oak, Cherrybark oak, Water oak, |
| | | Sycamore |
| | Goes tigrinus | Oak |
| | Neoclytus acuminatus | Ash, Hickory, Oak, Walnut, Birch, |
| | | Beech, Maple, Eastern |
| | | hophornbeam, Dogwood, |
| | | Persimmon, Redbud, Holly, |
| | | Hackberry, Black locust, |
| | | Honeylocust, Yellow-poplar, |
| | | Chestnut, Osage-orange, Sassafras, |
| | | Lilac, Mountain-mahogany, Pear, |
| | | Cherry, Plum, Peach, Apple, Elm, |
| | | Basswood, Sweetgum |
| | Neoptychodes trilineatus | Fig, Alder, Mulberry, Willow, Netleaf |
| | | hackberry |
| | Oberea ocellata | Sumac, Apple, Peach, Plum, Pear, |
| | | Currant, Blackberry |
| | Oberea tripunctata | Dogwood, Viburnum, Elm, |
| | | Sourwood, Blueberry, |
| | | Rhododendron, Azalea, Laurel, |
| | | Poplar, Willow, Mulberry |
| | Oncideres cingulata | Hickory, Pecan, Persimmon, Elm, |
| | | Sourwood, Basswood, Honeylocust, |
| | | Dogwood, Eucalyptus, Oak, |
| | | Hackberry, Maple, Fruit trees |
| | Saperda calcarata | Poplar |
| | Strophiona nitens | Chestnut, Oak, Hickory, Walnut, |
| | | Beech, Maple |
| Scolytidae | Corthylus columbianus | Maple, Oak, Yellow-poplar, Beech, |
| | | Boxelder, Sycamore, Birch, |
| | | Basswood, Chestnut, Elm |
| | Dendroctonus frontalis | Pine |
| | Dryocoetes betulae | Birch, Sweetgum, Wild cherry, |
| | | Beech, Pear |
| | Monarthrum fasciatum | Oak, Maple, Birch, Chestnut, |
| | | Sweetgum, Blackgum, Poplar, |
| | | Hickory, Mimosa, Apple, Peach, Pine |
| | Phloeotribus liminaris | Peach, Cherry, Plum, Black cherry, |
| | | Elm, Mulberry, Mountain-ash |
| | Pseudopityophthorus pruinosus | Oak, American beech, Black cherry, |
| | | Chickasaw plum, Chestnut, Maple, |
| | | Hickory, Hornbeam, Hophornbeam |
| Sesiidae | Paranthrene simulans | Oak, American chestnut |
| | Sannina uroceriformis | Persimmon |
| | Synanthedon exitiosa | Peach, Plum, Nectarine, Cherry, |
| | | Apricot, Almond, Black cherry |
| | Synanthedon pictipes | Peach, Plum, Cherry, Beach, Black |
| | | Cherry |
| | Synanthedon rubrofascia | Tupelo |
| | Synanthedon scitula | Dogwood, Pecan, Hickory, Oak, |
| | | Chestnut, Beech, Birch, Black cherry, |
| | | Elm, Mountain-ash, Viburnum, |
| | | Willow, Apple, Loquat, Ninebark, |
| | | Bayberry |
| | Vitacea polistiformis | Grape |

The present invention may be also used to control any insect pests that may be present in turfgrass, including for example beetles, caterpillars, fire ants, ground pearls, millipedes, sow bugs, mites, mole crickets, scales, mealybugs ticks, spittlebugs, southern chinch bugs and white grubs. The present invention may be used to control insect pests at various stages of their life cycle, including eggs, larvae, nymphs and adults.

In particular, the present invention may be used to control insect pests that feed on the roots of turfgrass including white grubs (such as Cyclocephala spp. (e.g. masked chafer, C. lurida), Rhizotrogus spp. (e.g. European chafer, R. majalis), Cotinus spp. (e.g. Green June beetle, C. nitida), Popillia spp. (e.g. Japanese beetle, P. japonica), Phyllophaga spp. (e.g. May/June beetle), Ataenius spp. (e.g. Black turfgrass ataenius, A. spretulus), Maladera spp. (e.g. Asiatic garden beetle, M. castanea) and Tomarus spp.), ground pearls (Margarodes spp.), mole crickets (tawny, southern, and short-winged; Scapteriscus spp., Gryllotalpa africana) and leatherjackets (European crane fly, Tipula spp.).

The present invention may also be used to control insect pests of turfgrass that are thatch dwelling, including armyworms (such as fall armyworm Spodoptera frugiperda, and common armyworm Pseudaletia unipuncta), cutworms, billbugs (Sphenophorus spp., such as S. venatus verstitus and S. parvulus), and sod webworms (such as Crambus spp. and the tropical sod webworm, Herpetogramma phaeopteralis).

The present invention may also be used to control insect pests of turfgrass that live above the ground and feed on the turfgrass leaves, including chinch bugs (such as southern chinch bugs, Blissus insularis), Bermudagrass mite (Eriophyes cynodoniensis), rhodesgrass mealybug (Antonina graminis), two-lined spittlebug (Propsapia bicincta), leafhoppers, cutworms (Noctuidae family), and greenbugs.

The present invention may also be used to control other pests of turfgrass such as red imported fire ants (Solenopsis invicta) that create ant mounds in turf.

In the hygiene sector, the compositions according to the invention are active against ectoparasites such as hard ticks, soft ticks, mange mites, harvest mites, flies (biting and licking), parasitic fly larvae, lice, hair lice, bird lice and fleas.

Examples of such parasites are:

Of the order Anoplurida: Haematopinus spp., Linognathus spp., Pediculus spp. and Phtirus spp., Solenopotes spp.

Of the order Mallophagida: Trimenopon spp., Menopon spp., Trinoton spp., Bovicola spp., Werneckiella spp., Lepikentron spp., Damalina spp., Trichodectes spp. and Felicola spp.

Of the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.

Of the order Siphonapterida, for example Pulex spp., Ctenocephalides spp., Xenopsylla spp., Ceratophyllus spp.

Of the order Heteropterida, for example Cimex spp., Triatoma spp., Rhodnius spp., Panstrongylus spp.

Of the order Blattarida, for example Blatta orientalis, Periplaneta americana, Blattelagermanica and Supella spp.

Of the subclass Acaria (Acarida) and the orders Meta- and Meso-stigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

Of the orders Actinedida (Prostigmata) and Acaridida (Astigmata), for example Acarapis spp., Cheyletiella spp., Ornithocheyletia spp., Myobia spp., Psorergatesspp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The compositions according to the invention are also suitable for protecting against insect infestation in the case of materials such as wood, textiles, plastics, adhesives, glues, paints, paper and card, leather, floor coverings and buildings.

The compositions according to the invention can be used, for example, against the following pests: beetles such as Hylotrupes bajulus, Chlorophorus pilosis, Anobium punctatum, Xestobium rufovillosum, Ptilinuspecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthesrugicollis, Xyleborus spec., Tryptodendron spec., Apate monachus, Bostrychus capucins, Heterobostrychus brunneus, Sinoxylon spec. and Dinoderus minutus, and also hymenopterans such as Sirex juvencus, Urocerus gigas, Urocerus gigas taignus and Urocerus augur, and termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus, and bristletails such as Lepisma saccharina.

The compounds according to the invention can be used as pesticidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethyl-hexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood N.J. (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.

The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 I/ha, especially from 10 to 1000 I/ha.

Formulation types include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP), a soluble granule (SG) or any technically feasible formulation in combination with agriculturally acceptable adjuvants.

The activity of the compositions according to the invention can be broadened considerably, and adapted to prevailing circumstances, by adding other insecticidally, acaricidally and/or fungicidally active ingredients. The mixtures of the compounds of formula (I) with other insecticidally, acaricidally and/or fungicidally active ingredients may also have further surprising advantages which can also be described, in a wider sense, as synergistic activity. For example, better tolerance by plants, reduced phytotoxicity, insects can be controlled in their different development stages or better behaviour during their production, for example during grinding or mixing, during their storage or during their use.

Suitable additions to active ingredients here are, for example, representatives of the following classes of active ingredients: organophosphorus compounds, nitrophenol derivatives, thioureas, juvenile hormones, formamidines, benzophenone derivatives, ureas, pyrrole derivatives, carbamates, pyrethroids, chlorinated hydrocarbons, acylureas, pyridylmethyleneamino derivatives, macrolides, neonicotinoids and Bacillus thuringiensis preparations.

The following mixtures of the compounds of formula (I) with active ingredients are preferred (the abbreviation “TX” means “one compound selected from the group consisting of the compounds described in Tables A-1 to A-240, in particular compounds A1 to A5:

an adjuvant selected from the group of substances consisting of petroleum oils (alternative name) (628)+TX,
an acaricide selected from the group of substances consisting of 1,1-bis(4-chlorophenyl)-2-ethoxyethanol (IUPAC name) (910)+TX, 2,4-dichlorophenyl benzenesulfonate (IUPAC/Chemical Abstracts name) (1059)+TX, 2-fluoro-N-methyl-N-1-naphthylacetamide (IUPAC name) (1295)+TX, 4-chlorophenyl phenyl sulfone (IUPAC name) (981)+TX, abamectin (1)+TX, acequinocyl (3)+TX, acetoprole [CCN]+TX, acrinathrin (9)+TX, aldicarb (16)+TX, aldoxycarb (863)+TX, alpha-cypermethrin (202)+TX, amidithion (870)+TX, amidoflumet [CCN]+TX, amidothioate (872)+TX, amiton (875)+TX, amiton hydrogen oxalate (875)+TX, amitraz (24)+TX, aramite (881)+TX, arsenous oxide (882)+TX, AVI 382 (compound code)+TX, AZ 60541 (compound code)+TX, azinphos-ethyl (44)+TX, azinphos-methyl (45)+TX, azobenzene (IUPAC name) (888)+TX, azocyclotin (46)+TX, azothoate (889)+TX, benomyl (62)+TX, benoxafos (alternative name) [CCN]+TX, benzoximate (71)+TX, benzyl benzoate (IUPAC name) [CCN]+TX, bifenazate (74)+TX, bifenthrin (76)+TX, binapacryl (907)+TX, brofenvalerate (alternative name)+TX, bromocyclen (918)+TX, bromophos (920)+TX, bromophos-ethyl (921)+TX, bromopropylate (94)+TX, buprofezin (99)+TX, butocarboxim (103)+TX, butoxycarboxim (104)+TX, butylpyridaben (alternative name)+TX, calcium polysulfide (IUPAC name) (111)+TX, camphechlor (941)+TX, carbanolate (943)+TX, carbaryl (115)+TX, carbofuran (118)+TX, carbophenothion (947)+TX, CGA 50′439 (development code) (125)+TX, chinomethionat (126)+TX, chlorbenside (959)+TX, chlordimeform (964)+TX, chlordimeform hydrochloride (964)+TX, chlorfenapyr (130)+TX, chlorfenethol (968)+TX, chlorfenson (970)+TX, chlorfensulfide (971)+TX, chlorfenvinphos (131)+TX, chlorobenzilate (975)+TX, chloromebuform (977)+TX, chloromethiuron (978)+TX, chloropropylate (983)+TX, chlorpyrifos (145)+TX, chlorpyrifos-methyl (146)+TX, chlorthiophos (994)+TX, cinerin 1 (696)+TX, cinerin II (696)+TX, cinerins (696)+TX, clofentezine (158)+TX, closantel (alternative name) [CCN]+TX, coumaphos (174)+TX, crotamiton (alternative name) [CCN]+TX, crotoxyphos (1010)+TX, cufraneb (1013)+TX, cyanthoate (1020)+TX, cyflumetofen (CAS Reg. No.: 400882-07-7)+TX, cyhalothrin (196)+TX, cyhexatin (199)+TX, cypermethrin (201)+TX, DCPM (1032)+TX, DDT (219)+TX, demephion (1037)+TX, demephion-O (1037)+TX, demephion-S (1037)+TX, demeton (1038)+TX, demeton-methyl (224)+TX, demeton-O (1038)+TX, demeton-O-methyl (224)+TX, demeton-S (1038)+TX, demeton-S-methyl (224)+TX, demeton-S-methylsulfon (1039)+TX, diafenthiuron (226)+TX, dialifos (1042)+TX, diazinon (227)+TX, dichlofluanid (230)+TX, dichlorvos (236)+TX, dicliphos (alternative name)+TX, dicofol (242)+TX, dicrotophos (243)+TX, dienochlor (1071)+TX, dimefox (1081)+TX, dimethoate (262)+TX, dinactin (alternative name) (653)+TX, dinex (1089)+TX, dinex-diclexine (1089)+TX, dinobuton (269)+TX, dinocap (270)+TX, dinocap-4 [CCN]+TX, dinocap-6 [CCN]+TX, dinocton (1090)+TX, dinopenton (1092)+TX, dinosulfon (1097)+TX, dinoterbon (1098)+TX, dioxathion (1102)+TX, diphenyl sulfone (IUPAC name) (1103)+TX, disulfiram (alternative name) [CCN]+TX, disulfoton (278)+TX, DNOC (282)+TX, dofenapyn (1113)+TX, doramectin (alternative name) [CCN]+TX, endosulfan (294)+TX, endothion (1121)+TX, EPN (297)+TX, eprinomectin (alternative name) [CCN]+TX, ethion (309)+TX, ethoate-methyl (1134)+TX, etoxazole (320)+TX, etrimfos (1142)+TX, fenazaflor (1147)+TX, fenazaquin (328)+TX, fenbutatin oxide (330)+TX, fenothiocarb (337)+TX, fenpropathrin (342)+TX, fenpyrad (alternative name)+TX, fenpyroximate (345)+TX, fenson (1157)+TX, fentrifanil (1161)+TX, fenvalerate (349)+TX, fipronil (354)+TX, fluacrypyrim (360)+TX, fluazuron (1166)+TX, flubenzimine (1167)+TX, flucycloxuron (366)+TX, flucythrinate (367)+TX, fluenetil (1169)+TX, flufenoxuron (370)+TX, flumethrin (372)+TX, fluorbenside (1174)+TX, fluvalinate (1184)+TX, FMC 1137 (development code) (1185)+TX, formetanate (405)+TX, formetanate hydrochloride (405)+TX, formothion (1192)+TX, formparanate (1193)+TX, gamma-HCH (430)+TX, glyodin (1205)+TX, halfenprox (424)+TX, heptenophos (432)+TX, hexadecyl cyclopropanecarboxylate (IUPAC/Chemical Abstracts name) (1216)+TX, hexythiazox (441)+TX, iodomethane (IUPAC name) (542)+TX, isocarbophos (alternative name) (473)+TX, isopropyl 0-(methoxyaminothiophosphoryl)salicylate (IUPAC name) (473)+TX, ivermectin (alternative name) [CCN]+TX, jasmolin 1 (696)+TX, jasmolin II (696)+TX, jodfenphos (1248)+TX, lindane (430)+TX, lufenuron (490)+TX, malathion (492)+TX, malonoben (1254)+TX, mecarbam (502)+TX, mephosfolan (1261)+TX, mesulfen (alternative name) [CCN]+TX, methacrifos (1266)+TX, methamidophos (527)+TX, methidathion (529)+TX, methiocarb (530)+TX, methomyl (531)+TX, methyl bromide (537)+TX, metolcarb (550)+TX, mevinphos (556)+TX, mexacarbate (1290)+TX, milbemectin (557)+TX, milbemycin oxime (alternative name) [CCN]+TX, mipafox (1293)+TX, monocrotophos (561)+TX, morphothion (1300)+TX, moxidectin (alternative name) [CCN]+TX, naled (567)+TX, NC-184 (compound code)+TX, NC-512 (compound code)+TX, nifluridide (1309)+TX, nikkomycins (alternative name) [CCN]+TX, nitrilacarb (1313)+TX, nitrilacarb 1:1 zinc chloride complex (1313)+TX, NNI-0101 (compound code)+TX, NNI-0250 (compound code)+TX, omethoate (594)+TX, oxamyl (602)+TX, oxydeprofos (1324)+TX, oxydisulfoton (1325)+TX, pp-DDT (219)+TX, parathion (615)+TX, permethrin (626)+TX, petroleum oils (alternative name) (628)+TX, phenkapton (1330)+TX, phenthoate (631)+TX, phorate (636)+TX, phosalone (637)+TX, phosfolan (1338)+TX, phosmet (638)+TX, phosphamidon (639)+TX, phoxim (642)+TX, pirimiphos-methyl (652)+TX, polychloroterpenes (traditional name) (1347)+TX, polynactins (alternative name) (653)+TX, proclonol (1350)+TX, profenofos (662)+TX, promacyl (1354)+TX, propargite (671)+TX, propetamphos (673)+TX, propoxur (678)+TX, prothidathion (1360)+TX, prothoate (1362)+TX, pyrethrin I (696)+TX, pyrethrin II (696)+TX, pyrethrins (696)+TX, pyridaben (699)+TX, pyridaphenthion (701)+TX, pyrimidifen (706)+TX, pyrimitate (1370)+TX, quinalphos (711)+TX, quintiofos (1381)+TX, R-1492 (development code) (1382)+TX, RA-17 (development code) (1383)+TX, rotenone (722)+TX, schradan (1389)+TX, sebufos (alternative name)+TX, selamectin (alternative name) [CCN]+TX, SI-0009 (compound code)+TX, sophamide (1402)+TX, spirodiclofen (738)+TX, spiromesifen (739)+TX, SSI-121 (development code) (1404)+TX, sulfiram (alternative name) [CCN]+TX, sulfluramid (750)+TX, sulfotep (753)+TX, sulfur (754)+TX, SZI-121 (development code) (757)+TX, tau-fluvalinate (398)+TX, tebufenpyrad (763)+TX, TEPP (1417)+TX, terbam (alternative name)+TX, tetrachlorvinphos (777)+TX, tetradifon (786)+TX, tetranactin (alternative name) (653)+TX, tetrasul (1425)+TX, thiafenox (alternative name)+TX, thiocarboxime (1431)+TX, thiofanox (800)+TX, thiometon (801)+TX, thioquinox (1436)+TX, thuringiensin (alternative name) [CCN]+TX, triamiphos (1441)+TX, triarathene (1443)+TX, triazophos (820)+TX, triazuron (alternative name)+TX, trichlorfon (824)+TX, trifenofos (1455)+TX, trinactin (alternative name) (653)+TX, vamidothion (847)+TX, vaniliprole [CCN] and YI-5302 (compound code)+TX,
an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347) and triphenyltin hydroxide (IUPAC name) (347)+TX, an anthelmintic selected from the group of substances consisting of abamectin (1)+TX, crufomate (1011)+TX, doramectin (alternative name) [CCN]+TX, emamectin (291)+TX, emamectin benzoate (291)+TX, eprinomectin (alternative name) [CCN]+TX, ivermectin (alternative name) [CCN]+TX, milbemycin oxime (alternative name) [CCN]+TX, moxidectin (alternative name) [CCN]+TX, piperazine [CCN]+TX, selamectin (alternative name) [CCN]+TX, spinosad (737) and thiophanate (1435)+TX, an avicide selected from the group of substances consisting of chloralose (127)+TX, endrin (1122)+TX, fenthion (346)+TX, pyridin-4-amine (IUPAC name) (23) and strychnine (745)+TX, a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen (alternative name) [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal (alternative name) [CCN]+TX, a biological agent selected from the group of substances consisting of Adoxophyes orana GV (alternative name) (12)+TX, Agrobacterium radiobacter (alternative name) (13)+TX, Amblyseius spp. (alternative name) (19)+TX, Anagrapha falcifera NPV (alternative name) (28)+TX, Anagrus atomus (alternative name) (29)+TX, Aphelinus abdominalis (alternative name) (33)+TX, Aphidius colemani (alternative name) (34)+TX, Aphidoletes aphidimyza (alternative name) (35)+TX, Autographa californica NPV (alternative name) (38)+TX, Bacillus firmus (alternative name) (48)+TX, Bacillus sphaericus Neide (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (alternative name) (53)+TX, Beauveria brongniartii (alternative name) (54)+TX, Chrysoperla carnea (alternative name) (151)+TX, Cryptolaemus montrouzieri (alternative name) (178)+TX, Cydia pomonella GV (alternative name) (191)+TX, Dacnusa sibirica (alternative name) (212)+TX, Diglyphus isaea (alternative name) (254)+TX, Encarsia formosa (scientific name) (293)+TX, Eretmocerus eremicus (alternative name) (300)+TX, Helicoverpa zea NPV (alternative name) (431)+TX, Heterorhabditis bacteriophora and H. megidis (alternative name) (433)+TX, Hippodamia convergens (alternative name) (442)+TX, Leptomastix dactylopii (alternative name) (488)+TX, Macrolophus caliginosus (alternative name) (491)+TX, Mamestra brassicae NPV (alternative name) (494)+TX, Metaphycus helvolus (alternative name) (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (alternative name) (575)+TX, Orius spp. (alternative name) (596)+TX, Paecilomyces fumosoroseus (alternative name) (613)+TX, Phytoseiulus persimilis (alternative name) (644)+TX, Spodoptera exigua multicapsid nuclear polyhedrosis virus (scientific name) (741)+TX, Steinernema bibionis (alternative name) (742)+TX, Steinernema carpocapsae (alternative name) (742)+TX, Steinernema feltiae (alternative name) (742)+TX, Steinernema glaseri (alternative name) (742)+TX, Steinernema riobrave (alternative name) (742)+TX, Steinernema riobravis (alternative name) (742)+TX, Steinernema scapterisci (alternative name) (742)+TX, Steinernema spp. (alternative name) (742)+TX, Trichogramma spp. (alternative name) (826)+TX, Typhlodromus occidentalis (alternative name) (844) and Verticillium lecanii (alternative name) (848)+TX, a soil sterilant selected from the group of substances consisting of iodomethane (IUPAC name) (542) and methyl bromide (537)+TX,
a chemosterilant selected from the group of substances consisting of apholate [CCN]+TX, bisazir (alternative name) [CCN]+TX, busulfan (alternative name) [CCN]+TX, diflubenzuron (250)+TX, dimatif (alternative name) [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron (alternative name) [CCN]+TX, tepa [CCN]+TX, thiohempa (alternative name) [CCN]+TX, thiotepa (alternative name) [CCN]+TX, tretamine (alternative name) [CCN] and uredepa (alternative name) [CCN]+TX, an insect pheromone selected from the group of substances consisting of (E)-dec-5-en-1-yl acetate with (E)-dec-5-en-1-ol (IUPAC name) (222)+TX, (E)-tridec-4-en-1-yl acetate (IUPAC name) (829)+TX, (E)-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-11-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-al (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin (alternative name) [CCN]+TX, brevicomin (alternative name) [CCN]+TX, codlelure (alternative name) [CCN]+TX, codlemone (alternative name) (167)+TX, cuelure (alternative name) (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure (alternative name) [CCN]+TX, ethyl 4-methyloctanoate (IUPAC name) (317)+TX, eugenol (alternative name) [CCN]+TX, frontalin (alternative name) [CCN]+TX, gossyplure (alternative name) (420)+TX, grandlure (421)+TX, grandlure I (alternative name) (421)+TX, grandlure II (alternative name) (421)+TX, grandlure III (alternative name) (421)+TX, grandlure IV (alternative name) (421)+TX, hexalure [CCN]+TX, ipsdienol (alternative name) [CCN]+TX, ipsenol (alternative name) [CCN]+TX, japonilure (alternative name) (481)+TX, lineatin (alternative name) [CCN]+TX, litlure (alternative name) [CCN]+TX, looplure (alternative name) [CCN]+TX, medlure [CCN]+TX, megatomoic acid (alternative name) [CCN]+TX, methyl eugenol (alternative name) (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure (alternative name) [CCN]+TX, oryctalure (alternative name) (317)+TX, ostramone (alternative name) [CCN]+TX, siglure [CCN]+TX, sordidin (alternative name) (736)+TX, sulcatol (alternative name) [CCN]+TX, tetradec-11-en-1-yl acetate (IUPAC name) (785)+TX, trimedlure (839)+TX, trimedlure A (alternative name) (839)+TX, trimedlure Bi (alternative name) (839)+TX, trimedlure B₂ (alternative name) (839)+TX, trimedlure C (alternative name) (839) and trunc-call (alternative name) [CCN]+TX,
an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethyltoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN] and picaridin [CCN]+TX,
an insecticide selected from the group of substances consisting of 1-dichloro-1-nitroethane (IUPAC/Chemical Abstracts name) (1058)+TX, 1,1-dichloro-2,2-bis(4-ethylphenyl)ethane (IUPAC name) (1056), +TX, 1,2-dichloropropane (IUPAC/Chemical Abstracts name) (1062)+TX, 1,2-dichloropropane with 1,3-dichloropropene (IUPAC name) (1063)+TX, 1-bromo-2-chloroethane (IUPAC/Chemical Abstracts name) (916)+TX, 2,2,2-trichloro-1-(3,4-dichlorophenyl)ethyl acetate (IUPAC name) (1451)+TX, 2,2-dichlorovinyl 2-ethylsulfinylethyl methyl phosphate (IUPAC name) (1066)+TX, 2-(1,3-dithiolan-2-yl)phenyl dimethylcarbamate (IUPAC/Chemical Abstracts name) (1109)+TX, 2-(2-butoxyethoxy)ethyl thiocyanate (IUPAC/Chemical Abstracts name) (935)+TX, 2-(4,5-dimethyl-1,3-dioxolan-2-yl)phenyl methylcarbamate (IUPAC/Chemical Abstracts name) (1084)+TX, 2-(4-chloro-3,5-xylyloxy)ethanol (IUPAC name) (986)+TX, 2-chlorovinyl diethyl phosphate (IUPAC name) (984)+TX, 2-imidazolidone (IUPAC name) (1225)+TX, 2-isovalerylindan-1,3-dione (IUPAC name) (1246)+TX, 2-methyl(prop-2-ynyl)aminophenyl methylcarbamate (IUPAC name) (1284)+TX, 2-thiocyanatoethyl laurate (IUPAC name) (1433)+TX, 3-bromo-1-chloroprop-1-ene (IUPAC name) (917)+TX, 3-methyl-1-phenylpyrazol-5-yl dimethylcarbamate (IUPAC name) (1283)+TX, 4-methyl(prop-2-ynyl)amino-3,5-xylyl methylcarbamate (IUPAC name) (1285)+TX, 5,5-dimethyl-3-oxocyclohex-1-enyl dimethylcarbamate (IUPAC name) (1085)+TX, abamectin (1)+TX, acephate (2)+TX, acetamiprid (4)+TX, acethion (alternative name) [CCN]+TX, acetoprole [CCN]+TX, acrinathrin (9)+TX, acrylonitrile (IUPAC name) (861)+TX, alanycarb (15)+TX, aldicarb (16)+TX, aldoxycarb (863)+TX, aldrin (864)+TX, allethrin (17)+TX, allosamidin (alternative name) [CCN]+TX, allyxycarb (866)+TX, alpha-cypermethrin (202)+TX, alpha-ecdysone (alternative name) [CCN]+TX, aluminium phosphide (640)+TX, amidithion (870)+TX, amidothioate (872)+TX, aminocarb (873)+TX, amiton (875)+TX, amiton hydrogen oxalate (875)+TX, amitraz (24)+TX, anabasine (877)+TX, athidathion (883)+TX, AVI 382 (compound code)+TX, AZ 60541 (compound code)+TX, azadirachtin (alternative name) (41)+TX, azamethiphos (42)+TX, azinphos-ethyl (44)+TX, azinphos-methyl (45)+TX, azothoate (889)+TX, Bacillus thuringiensis delta endotoxins (alternative name) (52)+TX, barium hexafluorosilicate (alternative name) [CCN]+TX, barium polysulfide (IUPAC/Chemical Abstracts name) (892)+TX, barthrin [CCN]+TX, Bayer 22/190 (development code) (893)+TX, Bayer 22408 (development code) (894)+TX, bendiocarb (58)+TX, benfuracarb (60)+TX, bensultap (66)+TX, beta-cyfluthrin (194)+TX, beta-cypermethrin (203)+TX, bifenthrin (76)+TX, bioallethrin (78)+TX, bioallethrin S-cyclopentenyl isomer (alternative name) (79)+TX, bioethanomethrin [CCN]+TX, biopermethrin (908)+TX, bioresmethrin (80)+TX, bis(2-chloroethyl) ether (IUPAC name) (909)+TX, bistrifluron (83)+TX, borax (86)+TX, brofenvalerate (alternative name)+TX, bromfenvinfos (914)+TX, bromocyclen (918)+TX, bromo-DDT (alternative name) [CCN]+TX, bromophos (920)+TX, bromophos-ethyl (921)+TX, bufencarb (924)+TX, buprofezin (99)+TX, butacarb (926)+TX, butathiofos (927)+TX, butocarboxim (103)+TX, butonate (932)+TX, butoxycarboxim (104)+TX, butylpyridaben (alternative name)+TX, cadusafos (109)+TX, calcium arsenate [CCN]+TX, calcium cyanide (444)+TX, calcium polysulfide (IUPAC name) (111)+TX, camphechlor (941)+TX, carbanolate (943)+TX, carbaryl (115)+TX, carbofuran (118)+TX, carbon disulfide (IUPAC/Chemical Abstracts name) (945)+TX, carbon tetrachloride (IUPAC name) (946)+TX, carbophenothion (947)+TX, carbosulfan (119)+TX, cartap (123)+TX, cartap hydrochloride (123)+TX, cevadine (alternative name) (725)+TX, chlorbicyclen (960)+TX, chlordane (128)+TX, chlordecone (963)+TX, chlordimeform (964)+TX, chlordimeform hydrochloride (964)+TX, chlorethoxyfos (129)+TX, chlorfenapyr (130)+TX, chlorfenvinphos (131)+TX, chlorfluazuron (132)+TX, chlormephos (136)+TX, chloroform [CCN]+TX, chloropicrin (141)+TX, chlorphoxim (989)+TX, chlorprazophos (990)+TX, chlorpyrifos (145)+TX, chlorpyrifos-methyl (146)+TX, chlorthiophos (994)+TX, chromafenozide (150)+TX, cinerin I (696)+TX, cinerin II (696)+TX, cinerins (696)+TX, cis-resmethrin (alternative name)+TX, cismethrin (80)+TX, clocythrin (alternative name)+TX, cloethocarb (999)+TX, closantel (alternative name) [CCN]+TX, clothianidin (165)+TX, copper acetoarsenite [CCN]+TX, copper arsenate [CCN]+TX, copper oleate [CCN]+TX, coumaphos (174)+TX, coumithoate (1006)+TX, crotamiton (alternative name) [CCN]+TX, crotoxyphos (1010)+TX, crufomate (1011)+TX, cryolite (alternative name) (177)+TX, CS 708 (development code) (1012)+TX, cyanofenphos (1019)+TX, cyanophos (184)+TX, cyanthoate (1020)+TX, cyclethrin [CCN]+TX, cycloprothrin (188)+TX, cyfluthrin (193)+TX, cyhalothrin (196)+TX, cypermethrin (201)+TX, cyphenothrin (206)+TX, cyromazine (209)+TX, cythioate (alternative name) [CCN]+TX, d-limonene (alternative name) [CCN]+TX, d-tetramethrin (alternative name) (788)+TX, DAEP (1031)+TX, dazomet (216)+TX, DDT (219)+TX, decarbofuran (1034)+TX, deltamethrin (223)+TX, demephion (1037)+TX, demephion-O (1037)+TX, demephion-S (1037)+TX, demeton (1038)+TX, demeton-methyl (224)+TX, demeton-O (1038)+TX, demeton-O-methyl (224)+TX, demeton-S (1038)+TX, demeton-S-methyl (224)+TX, demeton-S-methylsulphon (1039)+TX, diafenthiuron (226)+TX, dialifos (1042)+TX, diamidafos (1044)+TX, diazinon (227)+TX, dicapthon (1050)+TX, dichlofenthion (1051)+TX, dichlorvos (236)+TX, dicliphos (alternative name)+TX, dicresyl (alternative name) [CCN]+TX, dicrotophos (243)+TX, dicyclanil (244)+TX, dieldrin (1070)+TX, diethyl 5-methylpyrazol-3-yl phosphate (IUPAC name) (1076)+TX, diflubenzuron (250)+TX, dilor (alternative name) [CCN]+TX, dimefluthrin [CCN]+TX, dimefox (1081)+TX, dimetan (1085)+TX, dimethoate (262)+TX, dimethrin (1083)+TX, dimethylvinphos (265)+TX, dimetilan (1086)+TX, dinex (1089)+TX, dinex-diclexine (1089)+TX, dinoprop (1093)+TX, dinosam (1094)+TX, dinoseb (1095)+TX, dinotefuran (271)+TX, diofenolan (1099)+TX, dioxabenzofos (1100)+TX, dioxacarb (1101)+TX, dioxathion (1102)+TX, disulfoton (278)+TX, dithicrofos (1108)+TX, DNOC (282)+TX, doramectin (alternative name) [CCN]+TX, DSP (1115)+TX, ecdysterone (alternative name) [CCN]+TX, EI 1642 (development code) (1118)+TX, emamectin (291)+TX, emamectin benzoate (291)+TX, EMPC (1120)+TX, empenthrin (292)+TX, endosulfan (294)+TX, endothion (1121)+TX, endrin (1122)+TX, EPBP (1123)+TX, EPN (297)+TX, epofenonane (1124)+TX, eprinomectin (alternative name) [CCN]+TX, esfenvalerate (302)+TX, etaphos (alternative name) [CCN]+TX, ethiofencarb (308)+TX, ethion (309)+TX, ethiprole (310)+TX, ethoate-methyl (1134)+TX, ethoprophos (312)+TX, ethyl formate (IUPAC name) [CCN]+TX, ethyl-DDD (alternative name) (1056)+TX, ethylene dibromide (316)+TX, ethylene dichloride (chemical name) (1136)+TX, ethylene oxide [CCN]+TX, etofenprox (319)+TX, etrimfos (1142)+TX, EXD (1143)+TX, famphur (323)+TX, fenamiphos (326)+TX, fenazaflor (1147)+TX, fenchlorphos (1148)+TX, fenethacarb (1149)+TX, fenfluthrin (1150)+TX, fenitrothion (335)+TX, fenobucarb (336)+TX, fenoxacrim (1153)+TX, fenoxycarb (340)+TX, fenpirithrin (1155)+TX, fenpropathrin (342)+TX, fenpyrad (alternative name)+TX, fensulfothion (1158)+TX, fenthion (346)+TX, fenthion-ethyl [CCN]+TX, fenvalerate (349)+TX, fipronil (354)+TX, flonicamid (358)+TX, flubendiamide (CAS. Reg. No.: 272451-65-7)+TX, flucofuron (1168)+TX, flucycloxuron (366)+TX, flucythrinate (367)+TX, fluenetil (1169)+TX, flufenerim [CCN]+TX, flufenoxuron (370)+TX, flufenprox (1171)+TX, flumethrin (372)+TX, fluvalinate (1184)+TX, FMC 1137 (development code) (1185)+TX, fonofos (1191)+TX, formetanate (405)+TX, formetanate hydrochloride (405)+TX, formothion (1192)+TX, formparanate (1193)+TX, fosmethilan (1194)+TX, fospirate (1195)+TX, fosthiazate (408)+TX, fosthietan (1196)+TX, furathiocarb (412)+TX, furethrin (1200)+TX, gamma-cyhalothrin (197)+TX, gamma-HCH (430)+TX, guazatine (422)+TX, guazatine acetates (422)+TX, GY-81 (development code) (423)+TX, halfenprox (424)+TX, halofenozide (425)+TX, HCH (430)+TX, HEOD (1070)+TX, heptachlor (1211)+TX, heptenophos (432)+TX, heterophos [CCN]+TX, hexaflumuron (439)+TX, HHDN (864)+TX, hydramethylnon (443)+TX, hydrogen cyanide (444)+TX, hydroprene (445)+TX, hyquincarb (1223)+TX, imidacloprid (458)+TX, imiprothrin (460)+TX, indoxacarb (465)+TX, iodomethane (IUPAC name) (542)+TX, IPSP (1229)+TX, isazofos (1231)+TX, isobenzan (1232)+TX, isocarbophos (alternative name) (473)+TX, isodrin (1235)+TX, isofenphos (1236)+TX, isolane (1237)+TX, isoprocarb (472)+TX, isopropyl O-(methoxyaminothiophosphoryl)salicylate (IUPAC name) (473)+TX, isoprothiolane (474)+TX, isothioate (1244)+TX, isoxathion (480)+TX, ivermectin (alternative name) [CCN]+TX, jasmolin I (696)+TX, jasmolin II (696)+TX, jodfenphos (1248)+TX, juvenile hormone I (alternative name) [CCN]+TX, juvenile hormone II (alternative name) [CCN]+TX, juvenile hormone III (alternative name) [CCN]+TX, kelevan (1249)+TX, kinoprene (484)+TX, lambda-cyhalothrin (198)+TX, lead arsenate [CCN]+TX, lepimectin (CCN)+TX, leptophos (1250)+TX, lindane (430)+TX, lirimfos (1251)+TX, lufenuron (490)+TX, lythidathion (1253)+TX, m-cumenyl methylcarbamate (IUPAC name) (1014)+TX, magnesium phosphide (IUPAC name) (640)+TX, malathion (492)+TX, malonoben (1254)+TX, mazidox (1255)+TX, mecarbam (502)+TX, mecarphon (1258)+TX, menazon (1260)+TX, mephosfolan (1261)+TX, mercurous chloride (513)+TX, mesulfenfos (1263)+TX, metaflumizone (CCN)+TX, metam (519)+TX, metam-potassium (alternative name) (519)+TX, metam-sodium (519)+TX, methacrifos (1266)+TX, methamidophos (527)+TX, methanesulfonyl fluoride (IUPAC/Chemical Abstracts name) (1268)+TX, methidathion (529)+TX, methiocarb (530)+TX, methocrotophos (1273)+TX, methomyl (531)+TX, methoprene (532)+TX, methoquin-butyl (1276)+TX, methothrin (alternative name) (533)+TX, methoxychlor (534)+TX, methoxyfenozide (535)+TX, methyl bromide (537)+TX, methyl isothiocyanate (543)+TX, methylchloroform (alternative name) [CCN]+TX, methylene chloride [CCN]+TX, metofluthrin [CCN]+TX, metolcarb (550)+TX, metoxadiazone (1288)+TX, mevinphos (556)+TX, mexacarbate (1290)+TX, milbemectin (557)+TX, milbemycin oxime (alternative name) [CCN]+TX, mipafox (1293)+TX, mirex (1294)+TX, monocrotophos (561)+TX, morphothion (1300)+TX, moxidectin (alternative name) [CCN]+TX, naftalofos (alternative name) [CCN]+TX, naled (567)+TX, naphthalene (IUPAC/Chemical Abstracts name) (1303)+TX, NC-170 (development code) (1306)+TX, NC-184 (compound code)+TX, nicotine (578)+TX, nicotine sulfate (578)+TX, nifluridide (1309)+TX, nitenpyram (579)+TX, nithiazine (1311)+TX, nitrilacarb (1313)+TX, nitrilacarb 1:1 zinc chloride complex (1313)+TX, NNI-0101 (compound code)+TX, NNI-0250 (compound code)+TX, nornicotine (traditional name) (1319)+TX, novaluron (585)+TX, noviflumuron (586)+TX, 0-5-dichloro-4-iodophenyl O-ethyl ethylphosphonothioate (IUPAC name) (1057)+TX, O,O-diethyl O-4-methyl-2-oxo-2H-chromen-7-yl phosphorothioate (IUPAC name) (1074)+TX, O,O-diethyl O-6-methyl-2-propylpyrimidin-4-yl phosphorothioate (IUPAC name) (1075)+TX, O,O,O′,O′-tetrapropyl dithiopyrophosphate (IUPAC name) (1424)+TX, oleic acid (IUPAC name) (593)+TX, omethoate (594)+TX, oxamyl (602)+TX, oxydemeton-methyl (609)+TX, oxydeprofos (1324)+TX, oxydisulfoton (1325)+TX, pp′-DDT (219)+TX, para-dichlorobenzene [CCN]+TX, parathion (615)+TX, parathion-methyl (616)+TX, penfluron (alternative name) [CCN]+TX, pentachlorophenol (623)+TX, pentachlorophenyl laurate (IUPAC name) (623)+TX, permethrin (626)+TX, petroleum oils (alternative name) (628)+TX, PH 60-38 (development code) (1328)+TX, phenkapton (1330)+TX, phenothrin (630)+TX, phenthoate (631)+TX, phorate (636)+TX, phosalone (637)+TX, phosfolan (1338)+TX, phosmet (638)+TX, phosnichlor (1339)+TX, phosphamidon (639)+TX, phosphine (IUPAC name) (640)+TX, phoxim (642)+TX, phoxim-methyl (1340)+TX, pirimetaphos (1344)+TX, pirimicarb (651)+TX, pirimiphos-ethyl (1345)+TX, pirimiphos-methyl (652)+TX, polychlorodicyclopentadiene isomers (IUPAC name) (1346)+TX, polychloroterpenes (traditional name) (1347)+TX, potassium arsenite [CCN]+TX, potassium thiocyanate [CCN]+TX, prallethrin (655)+TX, precocene I (alternative name) [CCN]+TX, precocene II (alternative name) [CCN]+TX, precocene III (alternative name) [CCN]+TX, primidophos (1349)+TX, profenofos (662)+TX, profluthrin [CCN]+TX, promacyl (1354)+TX, promecarb (1355)+TX, propaphos (1356)+TX, propetamphos (673)+TX, propoxur (678)+TX, prothidathion (1360)+TX, prothiofos (686)+TX, prothoate (1362)+TX, protrifenbute [CCN]+TX, pymetrozine (688)+TX, pyraclofos (689)+TX, pyrazophos (693)+TX, pyresmethrin (1367)+TX, pyrethrin I (696)+TX, pyrethrin II (696)+TX, pyrethrins (696)+TX, pyridaben (699)+TX, pyridalyl (700)+TX, pyridaphenthion (701)+TX, pyrimidifen (706)+TX, pyrimitate (1370)+TX, pyriproxyfen (708)+TX, quassia (alternative name) [CCN]+TX, quinalphos (711)+TX, quinalphos-methyl (1376)+TX, quinothion (1380)+TX, quintiofos (1381)+TX, R-1492 (development code) (1382)+TX, rafoxanide (alternative name) [CCN]+TX, resmethrin (719)+TX, rotenone (722)+TX, RU 15525 (development code) (723)+TX, RU 25475 (development code) (1386)+TX, ryania (alternative name) (1387)+TX, ryanodine (traditional name) (1387)+TX, sabadilla (alternative name) (725)+TX, schradan (1389)+TX, sebufos (alternative name)+TX, selamectin (alternative name) [CCN]+TX, SI-0009 (compound code)+TX, SI-0205 (compound code)+TX, SI-0404 (compound code)+TX, SI-0405 (compound code)+TX, silafluofen (728)+TX, SN 72129 (development code) (1397)+TX, sodium arsenite [CCN]+TX, sodium cyanide (444)+TX, sodium fluoride (IUPAC/Chemical Abstracts name) (1399)+TX, sodium hexafluorosilicate (1400)+TX, sodium pentachlorophenoxide (623)+TX, sodium selenate (IUPAC name) (1401)+TX, sodium thiocyanate [CCN]+TX, sophamide (1402)+TX, spinosad (737)+TX, spiromesifen (739)+TX, spirotetrmat (CCN)+TX, sulcofuron (746)+TX, sulcofuron-sodium (746)+TX, sulfluramid (750)+TX, sulfotep (753)+TX, sulfuryl fluoride (756)+TX, sulprofos (1408)+TX, tar oils (alternative name) (758)+TX, tau-fluvalinate (398)+TX, tazimcarb (1412)+TX, TDE (1414)+TX, tebufenozide (762)+TX, tebufenpyrad (763)+TX, tebupirimfos (764)+TX, teflubenzuron (768)+TX, tefluthrin (769)+TX, temephos (770)+TX, TEPP (1417)+TX, terallethrin (1418)+TX, terbam (alternative name)+TX, terbufos (773)+TX, tetrachloroethane [CCN]+TX, tetrachlorvinphos (777)+TX, tetramethrin (787)+TX, theta-cypermethrin (204)+TX, thiacloprid (791)+TX, thiafenox (alternative name)+TX, thiamethoxam (792)+TX, thicrofos (1428)+TX, thiocarboxime (1431)+TX, thiocyclam (798)+TX, thiocyclam hydrogen oxalate (798)+TX, thiodicarb (799)+TX, thiofanox (800)+TX, thiometon (801)+TX, thionazin (1434)+TX, thiosultap (803)+TX, thiosultap-sodium (803)+TX, thuringiensin (alternative name) [CCN]+TX, tolfenpyrad (809)+TX, tralomethrin (812)+TX, transfluthrin (813)+TX, transpermethrin (1440)+TX, triamiphos (1441)+TX, triazamate (818)+TX, triazophos (820)+TX, triazuron (alternative name)+TX, trichlorfon (824)+TX, trichlormetaphos-3 (alternative name) [CCN]+TX, trichloronat (1452)+TX, trifenofos (1455)+TX, triflumuron (835)+TX, trimethacarb (840)+TX, triprene (1459)+TX, vamidothion (847)+TX, vaniliprole [CCN]+TX, veratridine (alternative name) (725)+TX, veratrine (alternative name) (725)+TX, XMC (853)+TX, xylylcarb (854)+TX, YI-5302 (compound code)+TX, zeta-cypermethrin (205)+TX, zetamethrin (alternative name)+TX, zinc phosphide (640)+TX, zolaprofos (1469) and ZXI 8901 (development code) (858)+TX, cyantraniliprole [736994-63-19+TX, chlorantraniliprole [500008-45-7]+TX, cyenopyrafen [560121-52-0]+TX, cyflumetofen [400882-07-7]+TX, pyrifluquinazon [337458-27-2]+TX, spinetoram [187166-40-1+187166-15-0]+TX, spirotetramat [203313-25-1]+TX, sulfoxaflor [946578-00-3]+TX, flufiprole [704886-18-0]+TX, meperfluthrin [915288-13-0]+TX, tetramethylfluthrin [84937-88-2]+TX, triflumezopyrim (disclosed in WO 2012/092115)+TX, fluxametamide (WO 2007/026965)+TX, epsilon-metofluthrin [240494-71-7]+TX, epsilon-momfluorothrin [1065124-65-3]+TX, fluazaindolizine [1254304-22-7]+TX, chloroprallethrin [399572-87-3]+TX, fluxametamide [928783-29-3]+TX, cyhalodiamide [1262605-53-7]+TX, tioxazafen [330459-31-9]+TX, broflanilide [1207727-04-5]+TX, flufiprole [704886-18-0]+TX, cyclaniliprole [1031756-98-5]+TX, tetraniliprole [1229654-66-3]+TX, guadipyr (described in WO2010/060231)+TX, cycloxaprid (described in WO2005/077934)+TX,
a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347) and triphenyltin hydroxide (IUPAC name) (347)+TX, pyriprole [394730-71-3]+TX,
a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-isopentenylaminopurine (alternative name) (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben (alternative name)+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, cytokinins (alternative name) (210)+TX, dazomet (216)+TX, DBCP (1045)+TX, DCIP (218)+TX, diamidafos (1044)+TX, dichlofenthion (1051)+TX, dicliphos (alternative name)+TX, dimethoate (262)+TX, doramectin (alternative name) [CCN]+TX, emamectin (291)+TX, emamectin benzoate (291)+TX, eprinomectin (alternative name) [CCN]+TX, ethoprophos (312)+TX, ethylene dibromide (316)+TX, fenamiphos (326)+TX, fenpyrad (alternative name)+TX, fensulfothion (1158)+TX, fosthiazate (408)+TX, fosthietan (1196)+TX, furfural (alternative name) [CCN]+TX, GY-81 (development code) (423)+TX, heterophos [CCN]+TX, iodomethane (IUPAC name) (542)+TX, isamidofos (1230)+TX, isazofos (1231)+TX, ivermectin (alternative name) [CCN]+TX, kinetin (alternative name) (210)+TX, mecarphon (1258)+TX, metam (519)+TX, metam-potassium (alternative name) (519)+TX, metam-sodium (519)+TX, methyl bromide (537)+TX, methyl isothiocyanate (543)+TX, milbemycin oxime (alternative name) [CCN]+TX, moxidectin (alternative name) [CCN]+TX, Myrothecium verrucaria composition (alternative name) (565)+TX, NC-184 (compound code)+TX, oxamyl (602)+TX, phorate (636)+TX, phosphamidon (639)+TX, phosphocarb [CCN]+TX, sebufos (alternative name)+TX, selamectin (alternative name) [CCN]+TX, spinosad (737)+TX, terbam (alternative name)+TX, terbufos (773)+TX, tetrachlorothiophene (IUPAC/Chemical Abstracts name) (1422)+TX, thiafenox (alternative name)+TX, thionazin (1434)+TX, triazophos (820)+TX, triazuron (alternative name)+TX, xylenols [CCN]+TX, YI-5302 (compound code) and zeatin (alternative name) (210)+TX, fluensulfone [318290-98-1]+TX,
a nitrification inhibitor selected from the group of substances consisting of potassium ethylxanthate [CCN] and nitrapyrin (580)+TX,
a plant activator selected from the group of substances consisting of acibenzolar (6)+TX, acibenzolar-S-methyl (6)+TX, probenazole (658) and Reynoutria sachalinensis extract (alternative name) (720)+TX,
a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (91)+TX, bromethalin (92)+TX, calcium cyanide (444)+TX, chloralose (127)+TX, chlorophacinone (140)+TX, cholecalciferol (alternative name) (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scilliroside (1390)+TX, sodium arsenite [CCN]+TX, sodium cyanide (444)+TX, sodium fluoro-acetate (735)+TX, strychnine (745)+TX, thallium sulfate [CCN]+TX, warfarin (851) and zinc phosphide (640)+TX,
a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (alternative name) (324)+TX, MB-599 (development code) (498)+TX, MGK 264 (development code) (296)+TX, piperonyl butoxide (649)+TX, piprotal (1343)+TX, propyl isomer (1358)+TX, S421 (development code) (724)+TX, sesamex (1393)+TX, sesasmolin (1394) and sulfoxide (1406)+TX,
an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX, pyridin-4-amine (IUPAC name) (23)+TX, thiram (804)+TX, trimethacarb (840)+TX, zinc naphthenate [CCN] and ziram (856)+TX,
a virucide selected from the group of substances consisting of imanin (alternative name) [CCN] and ribavirin (alternative name) [CCN]+TX,
a wound protectant selected from the group of substances consisting of mercuric oxide (512)+TX, octhilinone (590) and thiophanate-methyl (802)+TX,
and biologically active compounds selected from the group consisting of azaconazole (60207-31-0]+TX, bitertanol [70585-36-3]+TX, bromuconazole [116255-48-2]+TX, cyproconazole [94361-06-5]+TX, difenoconazole [119446-68-3]+TX, diniconazole [83657-24-3]+TX, epoxiconazole [106325-08-0]+TX, fenbuconazole [114369-43-6]+TX, fluquinconazole [136426-54-5]+TX, flusilazole [85509-19-9]+TX, flutriafol [76674-21-0]+TX, hexaconazole [79983-71-4]+TX, imazalil [35554-44-0]+TX, imibenconazole [86598-92-7]+TX, ipconazole [125225-28-7]+TX, metconazole [125116-23-6]+TX, myclobutanil [88671-89-0]+TX, pefurazoate [101903-30-4]+TX, penconazole [66246-88-6]+TX, prothioconazole [178928-70-6]+TX, pyrifenox [88283-41-4]+TX, prochloraz [67747-09-5]+TX, propiconazole [60207-90-1]+TX, simeconazole [149508-90-7]+TX, tebuconazole [107534-96-3]+TX, tetraconazole [112281-77-3]+TX, triadimefon [43121-43-3]+TX, triadimenol [55219-65-3]+TX, triflumizole [99387-89-0]+TX, triticonazole [131983-72-7]+TX, ancymidol [12771-68-5]+TX, fenarimol [60168-88-9]+TX, nuarimol [63284-71-9]+TX, bupirimate [41483-43-6]+TX, dimethirimol [5221-53-4]+TX, ethirimol [23947-60-6]+TX, dodemorph [1593-77-7]+TX, fenpropidine [67306-00-7]+TX, fenpropimorph [67564-91-4]+TX, spiroxamine [118134-30-8]+TX, tridemorph [81412-43-3]+TX, cyprodinil [121552-61-2]+TX, mepanipyrim [110235-47-7]+TX, pyrimethanil [53112-28-0]+TX, fenpiclonil [74738-17-3]+TX, fludioxonil [131341-86-1]+TX, benalaxyl [71626-11-4]+TX, furalaxyl [57646-30-7]+TX, metalaxyl [57837-19-1]+TX, R-metalaxyl [70630-17-0]+TX, ofurace [58810-48-3]+TX, oxadixyl [77732-09-3]+TX, benomyl [17804-35-2]+TX, carbendazim [10605-21-7]+TX, debacarb [62732-91-6]+TX, fuberidazole [3878-19-1]+TX, thiabendazole [148-79-8]+TX, chlozolinate [84332-86-5]+TX, dichlozoline [24201-58-9]+TX, iprodione [36734-19-7]+TX, myclozoline [54864-61-8]+TX, procymidone [32809-16-8]+TX, vinclozoline [50471-44-8]+TX, boscalid [188425-85-6]+TX, carboxin [5234-68-4]+TX, fenfuram [24691-80-3]+TX, flutolanil [66332-96-5]+TX, mepronil [55814-41-0]+TX, oxycarboxin [5259-88-1]+TX, penthiopyrad [183675-82-3]+TX, thifluzamide [130000-40-7]+TX, guazatine [108173-90-6]+TX, dodine [2439-10-3] [112-65-2] (free base)+TX, iminoctadine [13516-27-3]+TX, azoxystrobin [131860-33-8]+TX, dimoxystrobin [149961-52-4]+TX, enestroburin {Proc. BCPC, Int. Congr., Glasgow, 2003, 1, 93}+TX, fluoxastrobin [361377-29-9]+TX, kresoxim-methyl [143390-89-0]+TX, metominostrobin [133408-50-1]+TX, trifloxystrobin [141517-21-7]+TX, orysastrobin [248593-16-0]+TX, picoxystrobin [117428-22-5]+TX, pyraclostrobin [175013-18-0]+TX, ferbam [14484-64-1]+TX, mancozeb [8018-01-7]+TX, maneb [12427-38-2]+TX, metiram [9006-42-2]+TX, propineb [12071-83-9]+TX, thiram [137-26-8]+TX, zineb [12122-67-7]+TX, ziram [137-30-4]+TX, captafol [2425-06-1]+TX, captan [133-06-2]+TX, dichlofluanid [1085-98-9]+TX, fluoroimide [41205-21-4]+TX, folpet [133-07-3]+TX, tolylfluanid [731-27-1]+TX, bordeaux mixture [8011-63-0]+TX, copperhydroxid [20427-59-2]+TX, copperoxychlorid [1332-40-7]+TX, coppersulfat [7758-98-7]+TX, copperoxid [1317-39-1]+TX, mancopper [53988-93-5]+TX, oxine-copper [10380-28-6]+TX, dinocap [131-72-6]+TX, nitrothal-isopropyl [10552-74-6]+TX, edifenphos [17109-49-8]+TX, iprobenphos [26087-47-8]+TX, isoprothiolane [50512-35-1]+TX, phosdiphen [36519-00-3]+TX, pyrazophos [13457-18-6]+TX, tolclofos-methyl [57018-04-9]+TX, acibenzolar-S-methyl [135158-54-2]+TX, anilazine [101-05-3]+TX, benthiavalicarb [413615-35-7]+TX, blasticidin-S [2079-00-7]+TX, chinomethionat [2439-01-2]+TX, chloroneb [2675-77-6]+TX, chlorothalonil [1897-45-6]+TX, cyflufenamid [180409-60-3]+TX, cymoxanil [57966-95-7]+TX, dichlone [117-80-6]+TX, diclocymet [139920-32-4]+TX, diclomezine [62865-36-5]+TX, dicloran [99-30-9]+TX, diethofencarb [87130-20-9]+TX, dimethomorph [110488-70-5]+TX, SYP-LI90 (Flumorph) [211867-47-9]+TX, dithianon [3347-22-6]+TX, ethaboxam [162650-77-3]+TX, etridiazole [2593-15-9]+TX, famoxadone [131807-57-3]+TX, fenamidone [161326-34-7]+TX, fenoxanil [115852-48-7]+TX, fentin [668-34-8]+TX, ferimzone [89269-64-7]+TX, fluazinam [79622-59-6]+TX, fluopicolide [239110-15-7]+TX, flusulfamide [106917-52-6]+TX, fenhexamid [126833-17-8]+TX, fosetyl-aluminium [39148-24-8]+TX, hymexazol [10004-44-1]+TX, iprovalicarb [140923-17-7]+TX, IKF-916 (Cyazofamid) [120116-88-3]+TX, kasugamycin [6980-18-3]+TX, methasulfocarb [66952-49-6]+TX, metrafenone [220899-03-6]+TX, pencycuron [66063-05-6]+TX, phthalide [27355-22-2]+TX, polyoxins [11113-80-7]+TX, probenazole [27605-76-1]+TX, propamocarb [25606-41-1]+TX, proquinazid [189278-12-4]+TX, pyroquilon [57369-32-1]+TX, quinoxyfen [124495-18-7]+TX, quintozene [82-68-8]+TX, sulfur [7704-34-9]+TX, tiadinil [223580-51-6]+TX, triazoxide [72459-58-6]+TX, tricyclazole [41814-78-2]+TX, triforine [26644-46-2]+TX, validamycin [37248-47-8]+TX, zoxamide (RH7281) [156052-68-5]+TX, mandipropamid [374726-62-2]+TX, isopyrazam [881685-58-1]+TX, sedaxane [874967-67-6]+TX, 3-difluoromethyl-1-methyl-1H-pyrazole-4-carboxylic acid (9-dichloromethylene-1,2,3,4-tetrahydro-1,4-methano-naphthalen-5-yl)-amide (disclosed in WO 2007/048556)+TX, 3-difluoromethyl-1-methyl-1H-pyrazole-4-carboxylic acid (3′,4′,5′-trifluoro-biphenyl-2-yl)-amide (disclosed in WO 2006/087343)+TX, [(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H,11Hnaphtho[2,1-b]pyrano[3,4-e]pyran-4-yl]methyl-cyclopropanecarboxylate [915972-17-7]+TX,1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide [926914-55-8]+TX; lancotrione [1486617-21-3]+TX; florpyrauxifen [943832-81-3]+TX; ipfentrifluconazole[1417782-08-1]+TX; mefentrifluconazole [1417782-03-6]+TX; quinofumelin [861647-84-9]+TX; chloroprallethrin [399572-87-3]+TX; cyhalodiamide [1262605-53-7]+TX; fluazaindolizine [1254304-22-7]+TX; fluxametamide [928783-29-3]+TX; epsilon-metofluthrin [240494-71-7]+TX; epsilon-momfluorothrin [1065124-65-3]+TX; pydiflumetofen [1228284-64-7]+TX; kappa-bifenthrin [439680-76-9]+TX; broflanilide [1207727-04-5]+TX; dicloromezotiaz [1263629-39-5]+TX; dipymetitrone [16114-35-5]+TX; pyraziflumid [942515-63-1]+TX; and kappa-tefluthrin [391634-71-2]+TX; and
microbials including: Acinetobacter lwoffii+TX, Acremonium alternatum+TX+TX, Acremonium cephalosporium+TX+TX, Acremonium diospyri+TX, Acremonium obclavatum+TX, Adoxophyes orana granulovirus (AdoxGV) (Capex®)+TX, Agrobacterium radiobacter strain K84 (Galltrol-A®)+TX, Alternaria alternate+TX, Alternaria cassia+TX, Alternaria destruens (Smolder®)+TX, Ampelomyces quisqualis (AQ10®)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum+TX, (MicroAZ®+TX, TAZO B®)+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain CM-1+TX, Bacillus chitinosporus strain AQ746+TX, Bacillus licheniformis strain HB-2 (Biostart™ Rhizoboost®)+TX, Bacillus licheniformis strain 3086 (EcoGuard®+TX, Green Releaf®)+TX, Bacillus circulans+TX, Bacillus firmus (BioSafe®+TX, BioNem-WP®+TX, VOTiVO®)+TX, Bacillus firmus strain 1-1582+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain GB34 (Yield Shield®)+TX, Bacillus pumilus strain AQ717+TX, Bacillus pumilus strain QST 2808 (Sonata®+TX, Ballad Plus®)+TX, Bacillus spahericus (VectoLex®)+TX, Bacillus spp.+TX, Bacillus spp. strain AQ175+TX, Bacillus spp. strain AQ177+TX, Bacillus spp. strain AQ178+TX, Bacillus subtilis strain QST 713 (CEASE®+TX, Serenade®+TX, Rhapsody®)+TX, Bacillus subtilis strain QST 714 (JAZZ®)+TX, Bacillus subtilis strain AQ153+TX, Bacillus subtilis strain AQ743+TX, Bacillus subtilis strain QST3002+TX, Bacillus subtilis strain QST3004+TX, Bacillus subtilis var. amyloliquefaciens strain FZB24 (Taegro®+TX, Rhizopro®)+TX, Bacillus thuringiensis Cry 2Ae+TX, Bacillus thuringiensis CrylAb+TX, Bacillus thuringiensis aizawai GC 91 (Agree®)+TX, Bacillus thuringiensis israelensis (BMP123®+TX, Aquabac®+TX, VectoBac®)+TX, Bacillus thuringiensis kurstaki (Javelin®+TX, Deliver®+TX, CryMax®+TX, Bonide®+TX, Scutella WP®+TX, Turilav WP®+TX, Astuto®+TX, Dipel WP®+TX, Biobit®+TX, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain BD #32+TX, Bacillus thuringiensis strain AQ52+TX, Bacillus thuringiensis var. aizawai (XenTari®+TX, DiPel®)+TX, bacteria spp. (GROWMEND®+TX, GROWSWEET®+TX, Shootup®)+TX, bacteriophage of Clavipacter michiganensis (AgriPhage®)+TX, Bakflor®+TX, Beauveria bassiana (Beaugenic®+TX, Brocaril WP®)+TX, Beauveria bassiana GHA (Mycotrol ES®+TX, Mycotrol O®+TX, BotaniGuard®)+TX, Beauveria brongniartii (Engerlingspilz®+TX, Schweizer Beauveria®+TX, Melocont®)+TX, Beauveria spp.+TX, Botrytis cineria+TX, Bradyrhizobium japonicum (TerraMax®)+TX, Brevibacillus brevis+TX, Bacillus thuringiensis tenebrionis (Novodor®)+TX, BtBooster+TX, Burkholderia cepacia (Deny®+TX, Intercept®+TX, Blue Circle®)+TX, Burkholderia gladii+TX, Burkholderia gladioli+TX, Burkholderia spp.+TX, Canadian thistle fungus (CBH Canadian Bioherbicide®)+TX, Candida butyri+TX, Candida famata+TX, Candida fructus+TX, Candida glabrata+TX, Candida guilliermondii+TX, Candida melibiosica+TX, Candida oleophila strain O+TX, Candida parapsilosis+TX, Candida pelliculosa+TX, Candida pulcherrima+TX, Candida reukaufii+TX, Candida saitoana (Bio-Coat®+TX, Biocure®)+TX, Candida sake+TX, Candida spp.+TX, Candida tenius+TX, Cedecea dravisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium cladosporioides+TX, Cladosporium oxysporum+TX, Cladosporium chlorocephalum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS®)+TX, Cryptococcus humicola+TX, Cryptococcus infirmo-miniatus+TX, Cryptococcus laurentii+TX, Cryptophlebia leucotreta granulovirus (Cryptex®)+TX, Cupriavidus campinensis+TX, Cydia pomonella granulovirus (CYD-X®)+TX, Cydia pomonella granulovirus (Madex®+TX, Madex Plus®+TX, Madex Max/Carpovirusine®)+TX, Cylindrobasidium laeve (Stumpout®)+TX, Cylindrocladium+TX, Debaryomyces hansenii+TX, Drechslera hawaiinensis+TX, Enterobacter cloacae+TX, Enterobacteriaceae+TX, Entomophtora virulenta (Vektor®)+TX, Epicoccum nigrum+TX, Epicoccum purpurascens+TX, Epicoccum spp.+TX, Filobasidium floriforme+TX, Fusarium acuminatum+TX, Fusarium chlamydosporum+TX, Fusarium oxysporum (Fusaclean®/Biofox C®)+TX, Fusarium proliferatum+TX, Fusarium spp.+TX, Galactomyces geotrichum+TX, Gliocladium catenulatum (Primastop®+TX, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isoflavone—formononetin (Myconate®)+TX, Kloeckera apiculata+TX, Kloeckera spp.+TX, Lagenidium giganteum (Laginex®)+TX, Lecanicillium longisporum (Vertiblast®)+TX, Lecanicillium muscarium (Vertikil®)+TX, Lymantria Dispar nucleopolyhedrosis virus (Disparvirus®)+TX, Marinococcus halophilus+TX, Meira geulakonigii+TX, Metarhizium anisopliae (Met52®)+TX, Metarhizium anisopliae (Destruxin WP®)+TX, Metschnikowia fruticola (Shemer®)+TX, Metschnikowia pulcherrima+TX, Microdochium dimerum (Antibot®)+TX, Micromonospora coerulea+TX, Microsphaeropsis ochracea+TX, Muscodor albus 620 (Muscudor®)+TX, Muscodor roseus strain A3-5+TX, Mycorrhizae spp. (AMykor®+TX, Root Maximizer®)+TX, Myrothecium verrucaria strain AARC-0255 (DiTera®)+TX, BROS PLUS®+TX, Ophiostoma piliferum strain D97 (Sylvanex®)+TX, Paecilomyces farinosus+TX, Paecilomyces fumosoroseus (PFR-97®+TX, PreFeRal®)+TX, Paecilomyces linacinus (Biostat WP®)+TX, Paecilomyces lilacinus strain 251 (MeloCon WG®)+TX, Paenibacillus polymyxa+TX, Pantoea agglomerans (BlightBan C9-1®)+TX, Pantoea spp.+TX, Pasteuria spp. (Econem®)+TX, Pasteuria nishizawae+TX, Penicillium aurantiogriseum+TX, Penicillium billai (Jumpstart®+TX, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeal®)+TX, Phytophthora cryptogea+TX, Phytophthora palmivora (Devine®)+TX, Pichia anomala+TX, Pichia guilermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlororaphis (AtEze®)+TX, Pseudomonas corrugate+TX, Pseudomonas fluorescens strain A506 (BlightBan A506®)+TX, Pseudomonas putida+TX, Pseudomonas reactans+TX, Pseudomonas spp.+TX, Pseudomonas syringae (Bio-Save®)+TX, Pseudomonas viridiflava+TX, Pseudomons fluorescens (Zequanox®)+TX, Pseudozyma flocculosa strain PF-A22 UL (Sporodex L®)+TX, Puccinia canaliculata+TX, Puccinia thlaspeos (Wood Warrior®)+TX, Pythium paroecandrum+TX, Pythium oligandrum (Polygandron®+TX, Polyversum®)+TX, Pythium periplocum+TX, Rhanella aquatilis+TX, Rhanella spp.+TX, Rhizobia (Dormal®+TX, Vault®)+TX, Rhizoctonia+TX, Rhodococcus globerulus strain AQ719+TX, Rhodosporidium diobovatum+TX, Rhodosporidium toruloides+TX, Rhodotorula spp.+TX, Rhodotorula glutinis+TX, Rhodotorula graminis+TX, Rhodotorula mucilagnosa+TX, Rhodotorula rubra+TX, Saccharomyces cerevisiae+TX, Salinococcus roseus+TX, Sclerotinia minor+TX, Sclerotinia minor (SARRITOR®)+TX, Scytalidium spp.+TX, Scytalidium uredinicola+TX, Spodoptera exigua nuclear polyhedrosis virus (Spod-X®+TX, Spexit®)+TX, Serratia marcescens+TX, Serratia plymuthica+TX, Serratia spp.+TX, Sordaria fimicola+TX, Spodoptera littoralis nucleopolyhedrovirus (Littovir®)+TX, Sporobolomyces roseus+TX, Stenotrophomonas maltophilia+TX, Streptomyces ahygroscopicus+TX, Streptomyces albaduncus+TX, Streptomyces exfoliates+TX, Streptomyces galbus+TX, Streptomyces griseoplanus+TX, Streptomyces griseoviridis (Mycostop®)+TX, Streptomyces lydicus (Actinovate®)+TX, Streptomyces lydicus WYEC-108 (ActinoGrow®)+TX, Streptomyces violaceus+TX, Tilletiopsis minor+TX, Tilletiopsis spp.+TX, Trichoderma asperellum (T34 Biocontrol®)+TX, Trichoderma gamsii (Tenet®)+TX, Trichoderma atroviride (Plantmate®)+TX, Trichoderma hamatum TH 382+TX, Trichoderma harzianum rifai (Mycostar®)+TX, Trichoderma harzianum T-22 (Trianum-P®+TX, PlantShield HC®+TX, RootShield®+TX, Trianum-G®)+TX, Trichoderma harzianum T-39 (Trichodex®)+TX, Trichoderma inhamatum+TX, Trichoderma koningii+TX, Trichoderma spp. LC 52 (Sentinel®)+TX, Trichoderma lignorum+TX, Trichoderma longibrachiatum+TX, Trichoderma polysporum (Binab T®)+TX, Trichoderma taxi+TX, Trichoderma virens+TX, Trichoderma virens (formerly Gliocladium virens GL-21) (SoilGuard®)+TX, Trichoderma viride+TX, Trichoderma viride strain ICC 080 (Remedier®)+TX, Trichosporon pullulans+TX, Trichosporon spp.+TX, Trichothecium spp.+TX, Trichothecium roseum+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural II®)+TX, various fungi (Millennium Microbes®)+TX, Verticillium chlamydosporium+TX, Verticillium lecanii (Mycotal®+TX, Vertalec®)+TX, Vip3Aa20 (VIPtera®)+TX, Virgibaclillus marismortui+TX, Xanthomonas campestris pv. Poae (Camperico®)+TX, Xenorhabdus bovienii+TX, Xenorhabdus nematophilus; and plant extracts including: pine oil (Retenol®)+TX, azadirachtin (Plasma Neem Oil®+TX, AzaGuard®+TX, MeemAzal®+TX, Molt-X®+TX, Botanical IGR (Neemazad®+TX, Neemix®)+TX, canola oil (Lilly Miller Vegol®)+TX, Chenopodium ambrosioides near ambrosioides (Requiem®)+TX, Chrysanthemum extract (Crisant®)+TX, extract of neem oil (Trilogy®)+TX, essentials oils of Labiatae (Botania®)+TX, extracts of clove rosemary peppermint and thyme oil (Garden insect Killer®)+TX, Glycinebetaine (Greenstim®)+TX, garlic+TX, lemongrass oil (GreenMatch®)+TX, neem oil+TX, Nepeta cataria (Catnip oil)+TX, Nepeta catarina+TX, nicotine+TX, oregano oil (MossBuster®)+TX, Pedaliaceae oil (Nematon®)+TX, pyrethrum+TX, Quillaja saponaria (NemaQ®)+TX, Reynoutria sachalinensis (Regalia®+TX, Sakalia®)+TX, rotenone (Eco Roten®)+TX, Rutaceae plant extract (Soleo®)+TX, soybean oil (Ortho Ecosense®)+TX, tea tree oil (Timorex Gold®)+TX, thymus oil+TX, AGNIQUE® MMF+TX, BugOil®+TX, mixture of rosemary sesame pepermint thyme and cinnamon extracts (EF 300®)+TX, mixture of clove rosemary and peppermint extract (EF 400®)+TX, mixture of clove pepermint garlic oil and mint (Soil Shot®)+TX, kaolin (Screen®)+TX, storage glucam of brown algae (Laminarin®); and
pheromones including: blackheaded fireworm pheromone (3M Sprayable Blackheaded Fireworm Pheromone®)+TX, Codling Moth Pheromone (Paramount dispenser-(CM)/Isomate C-Plus®)+TX, Grape Berry Moth Pheromone (3M MEC-GBM Sprayable Pheromone®)+TX, Leafroller pheromone (3M MEC—LR Sprayable Pheromone®)+TX, Muscamone (Snip7 Fly Bait®+TX, Starbar Premium Fly Bait®)+TX, Oriental Fruit Moth Pheromone (3M oriental fruit moth sprayable Pheromone®)+TX, Peachtree Borer Pheromone (Isomate-P®)+TX, Tomato Pinworm Pheromone (3M Sprayable Pheromone®)+TX, Entostat powder (extract from palm tree) (Exosex CM®)+TX, (E+TX,Z+TX,Z)-3+TX,8+TX, 11 Tetradecatrienyl acetate+TX, (Z+TX,Z+TX,E)-7+TX,11+TX,13-Hexadecatrienal+TX, (E+TX,Z)-7+TX,9-Dodecadien-1-yl acetate+TX, 2-Methyl-1-butanol+TX, Calcium acetate+TX, Scenturion®+TX, Biolure®+TX, Check-Mate®+TX, Lavandulyl senecioate; and macrobials including: Aphelinus abdominalis+TX, Aphidius ervi (Aphelinus-System®)+TX, Acerophagus papaya+TX, Adalia bipunctata (Adalia-System®)+TX, Adalia bipunctata (Adaline®)+TX, Adalia bipunctata (Aphidalia®)+TX, Ageniaspis citricola+TX, Ageniaspis fuscicollis+TX, Amblyseius andersoni (Anderline®+TX, Andersoni-System®)+TX, Amblyseius californicus (Amblyline®+TX, Spical®)+TX, Amblyseius cucumeris (Thripex®+TX, Bugline cucumeris®)+TX, Amblyseius fallacis (Fallacis®)+TX, Amblyseius swirskii (Bugline Swirskii®+TX, Swirskii-Mite®)+TX, Amblyseius womersleyi (WomerMite®)+TX, Amitus hesperidum+TX, Anagrus atomus+TX, Anagyrus fusciventris+TX, Anagyrus kamali+TX, Anagyrus loecki+TX, Anagyrus pseudococci (Citripar®)+TX, Anicetus benefices+TX, Anisopteromalus calandrae+TX, Anthocoris nemoralis (Anthocoris-System®)+TX, Aphelinus abdominalis (Apheline®+TX, Aphiline®)+TX, Aphelinus asychis+TX, Aphidius colemani (Aphipar®)+TX, Aphidius ervi (Ervipar®)+TX, Aphidius gifuensis+TX, Aphidius matricariae (Aphipar-M®)+TX, Aphidoletes aphidimyza (Aphidend®)+TX, Aphidoletes aphidimyza (Aphidoline®)+TX, Aphytis lingnanensis+TX, Aphytis melinus+TX, Aprostocetus hagenowii+TX, Atheta coriaria (Staphyline®)+TX, Bombus spp.+TX, Bombus terrestris (Natupol Beehive®)+TX, Bombus terrestris (Beeline®+TX, Tripol®)+TX, Cephalonomia stephanoderis+TX, Chilocorus nigritus+TX, Chrysoperla carnea (Chrysoline®)+TX, Chrysoperla carnea (Chrysopa®)+TX, Chrysoperla rufilabris+TX, Cirrospilus ingenuus+TX, Cirrospilus quadristriatus+TX, Citrostichus phyllocnistoides+TX, Closterocerus chamaeleon+TX, Closterocerus spp.+TX, Coccidoxenoides perminutus (Planopar®)+TX, Coccophagus cowperi+TX, Coccophagus lycimnia+TX, Cotesia flavipes+TX, Cotesia plutellae+TX, Cryptolaemus montrouzieri (Cryptobug®+TX, Cryptoline®)+TX, Cybocephalus nipponicus+TX, Dacnusa sibirica+TX, Dacnusa sibirica (Minusa®)+TX, Diglyphus isaea (Diminex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrtus aligarhensis+TX, Diglyphus isaea+TX, Diglyphus isaea (Miglyphus®+TX, Digline®)+TX, Dacnusa sibirica (DacDigline®+TX, Minex®)+TX, Diversinervus spp.+TX, Encarsia citrina+TX, Encarsia formosa (Encarsia max@+TX, Encarline®+TX, En-Strip®)+TX, Eretmocerus eremicus (Enermix®)+TX, Encarsia guadeloupae+TX, Encarsia haitiensis+TX, Episyrphus balteatus (Syrphidend®)+TX, Eretmoceris siphonini+TX, Eretmocerus californicus+TX, Eretmocerus eremicus (Ercal®+TX, Eretline e®)+TX, Eretmocerus eremicus (Bemimix®)+TX, Eretmocerus hayati+TX, Eretmocerus mundus (Bemipar®+TX, Eretline m®)+TX, Eretmocerus siphonini+TX, Exochomus quadripustulatus+TX, Feltiella acarisuga (Spidend®)+TX, Feltiella acarisuga (Feltiline®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis spp. (Lawn Patrol®)+TX, Heterorhabditis bacteriophora (NemaShield HB®+TX, Nemaseek®+TX, Terranem-Nam®+TX, Terranem®+TX, Larvanem®+TX, B-Green®+TX, NemAttack®+TX, Nematop®)+TX, Heterorhabditis megidis (Nemasys H®+TX, BioNem H®+TX, Exhibitline hm®+TX, Larvanem-M®)+TX, Hippodamia convergens+TX, Hypoaspis aculeifer (Aculeifer-System®+TX, Entomite-A®)+TX, Hypoaspis miles (Hypoline m®+TX, Entomite-M®)+TX, Lbalia leucospoides+TX, Lecanoideus floccissimus+TX, Lemophagus errabundus+TX, Leptomastidea abnormis+TX, Leptomastix dactylopii (Leptopar®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®+TX, Macroline c®+TX, Mirical®)+TX, Mesoseiulus longipes+TX, Metaphycus flavus+TX, Metaphycus lounsburyi+TX, Micromus angulatus (Milacewing®)+TX, Microterys flavus+TX, Muscidifurax raptorellus and Spalangia cameroni (Biopar®)+TX, Neodryinus typhlocybae+TX, Neoseiulus californicus+TX, Neoseiulus cucumeris (THRYPEX®)+TX, Neoseiulus fallacis+TX, Nesideocoris tenuis (NesidioBug®+TX, Nesibug®)+TX, Ophyra aenescens (Biofly®)+TX, Orius insidiosus (Thripor-®+TX, Oriline i®)+TX, Orius laevigatus (Thripor-L®+TX, Oriline I®)+TX, Orius majusculus (Oriline m®)+TX, Orius strigicollis (Thripor-S®)+TX, Pauesia juniperorum+TX, Pediobius foveolatus+TX, Phasmarhabditis hermaphrodita (Nemaslug®)+TX, Phymastichus coffea+TX, Phytoseiulus macropilus+TX, Phytoseiulus persimilis (Spidex®+TX, Phytoline p®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®+TX, Millenium®+TX, BioNem C®+TX, NemAttack®+TX, Nemastar®+TX, Capsanem®)+TX, Steinernema feltiae (NemaShield®+TX, Nemasys F®+TX, BioNem F®+TX, Steinernema-System®+TX, NemAttack®+TX, Nemaplus®+TX, Exhibitline sf®+TX, Scia-Rid®+TX, Entonem®)+TX, Steinernema kraussei (Nemasys L®+TX, BioNem L®+TX, Exhibitline srb®)+TX, Steinernema riobrave (BioVector®+TX, BioVektor®)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer+TX, Thripobius semiluteus+TX, Torymus sinensis+TX, Trichogramma brassicae (Tricholine b®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimpla stemmator; and
other biologicals including: abscisic acid+TX, bioSea®+TX, Chondrostereum purpureum (Chontrol Paste®)+TX, Colletotrichum gloeosporioides (Collego®)+TX, Copper Octanoate (Cueva®)+TX, Delta traps (Trapline d®)+TX, Erwinia amylovora (Harpin) (ProAct®+TX, Ni-HIBIT Gold CST®)+TX, Ferri-phosphate (Ferramol®)+TX, Funnel traps (Trapline y®)+TX, Gallex®+TX, Grower's Secret®+TX, Homo-brassonolide+TX, Iron Phosphate (Lilly Miller Worry Free Ferramol Slug & Snail Bait®)+TX, MCP hail trap (Trapline f®)+TX, Microctonus hyperodae+TX, Mycoleptodiscus terrestris (Des-X®)+TX, BioGain®+TX, Aminomite®+TX, Zenox®+TX, Pheromone trap (Thripline ams®)+TX, potassium bicarbonate (MilStop®)+TX, potassium salts of fatty acids (Sanova®)+TX, potassium silicate solution (Sil-Matrix®)+TX, potassium iodide+potassiumthiocyanate (Enzicur®)+TX, SuffOil-X®+TX, Spider venom+TX, Nosema locustae (Semaspore Organic Grasshopper Control®)+TX, Sticky traps (Trapline YF®+TX, Rebell Amarillo®)+TX and Traps (Takitrapline y+b®)+TX.

The references in brackets behind the active ingredients, e.g. [3878-19-1] refer to the Chemical Abstracts Registry number. The above described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright © 1995-2004]; for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “develoment code” is used or, if neither one of those designations nor a “common name” is used, an “alternative name” is employed. “CAS Reg. No” means the Chemical Abstracts Registry Number.

The active ingredient mixture of the compounds of formula (I) selected from Tables 1 to 96 (including 1a to 96a to 1 m to 96m) and Table A with active ingredients described above comprises a compound selected from Tables 1 to 96 (including 1a to 96a to 1m to 96m) and Table A and an active ingredient as described above preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound of formula (I) selected from Tables 1 to 96 (including 1a to 96a to 1m to 96m) and Table A and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula (I) selected from Tables 1 to 96 (including 1a to 96a to 1m to 96m) and Table A and the active ingredients as described above is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is generally 1 to 2000 g of active ingredient per hectare, in particular 10 to 1000 g/ha, preferably 10 to 600 g/ha.

A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

The compounds of the invention and compositions thereof are also be suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest/fungi to be controlled and are generally between 1 to 200 grams per 100 kg of seeds, preferably between 5 to 150 grams per 100 kg of seeds, such as between 10 to 100 grams per 100 kg of seeds.

The term seed embraces seeds and plant propagules of all kinds including but not limited to true seeds, seed pieces, suckers, corns, bulbs, fruit, tubers, grains, rhizomes, cuttings, cut shoots and the like and means in a preferred embodiment true seeds.

The present invention also comprises seeds coated or treated with or containing a compound of formula (I). The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula (I). Further, it is hereby made available, a composition comprising a plant propagation material treated with a compound of formula (I).

Seed treatment comprises all suitable seed treatment techniques known in the art, such as seed dressing, seed coating, seed dusting, seed soaking and seed pelleting. The seed treatment application of the compound formula (I) can be carried out by any known methods, such as spraying or by dusting the seeds before sowing or during the sowing/planting of the seeds.

The invention further relates to a pesticidal composition, which comprises at least one compound of formula (I) according to this invention or at least one compound of formula (Ib) or, where appropriate, a tautomer thereof, in each case in free form or in agrochemically utilizable salt form, as active ingredient.

The invention further relates to a method for controlling pests, which comprises applying a composition according to the invention to the pests or their environment preferably with the exception of a method for treatment of the human or animal body by surgery or therapy and diagnostic methods practised on the human or animal body.

The invention further relates to a method for the protection of plant propagation material from the attack by pests, which comprises treating the propagation material or the site, where the propagation material is planted, with a composition comprising a compound according to this invention or with a compound according to this invention. The invention further relates to a plant propagation material treated with the pesticidal composition comprising a compound according to this invention or with a compound according to this invention.

The Examples which follow serve to illustrate the invention.

The following Examples illustrate, but do not limit, the invention.

The compounds of the invention can be distinguished from known compounds by virtue of greater efficacy at low application rates, which can be verified by the person skilled in the art using the experimental procedures outlined in the Examples, using lower application rates if necessary, for example 50 ppm, 12.5 ppm, 6 ppm, 3 ppm, 1.5 ppm, 0.8 ppm, 0.4 ppm, 0.2 ppm, 0.1 ppm or even at lower concentrations.

### PREPARATORY EXAMPLES

“Mp” means melting point in ° C. Free radicals represent methyl groups. ¹H and ¹⁹F NMR measurements were recorded on Brucker 400 MHz or 300 MHz spectrometers, chemical shifts are given in ppm relevant to a TMS standard. Spectra measured in deuterated solvents as indicated.

The compounds from this application were prepared using standard coupling conditions with BOP—Cl as a coupling agent, following the generic reaction scheme below:

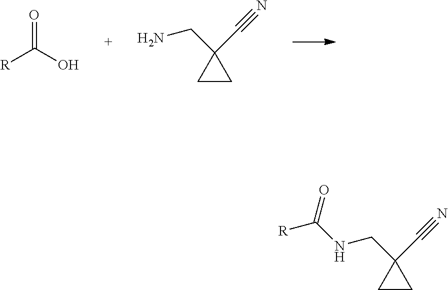

The following LC-MS method was used to characterize the compounds:

Method A
Waters Aquity UPLC-MS using a Sample Organizer with Sample Manager FTN, H-class QSM,
Column Manager, 2× Column Manager Aux, photodiode array, ELSD and SQD 2 equipped with
a Waters HSS T3 C18 column (column length 30 mm, internal diameter of column 2.1 mm,
particle size 1.8 micron). The analysis was conducted using a two minute run time, according to
the following gradient table:

| Time (mins) | Solvent A (%) | Solvent B (%) | Flow (ml/mn) |
|---|---|---|---|
| 0 | 95 | 5 | 0.7 |
| 1.75 | 0 | 100 | 0.7 |
| 1.76 | 0 | 100 | 0.7 |
| 2 | 0 | 5 | 0.7 |
| 2.01 | 95 | 5 | 0.7 |
| 2.11 | 95 | 5 | 0.7 |

Solvent A: H₂O with 0.05% trifluoroacetic acid (TFA)
Solvent B: CH₃CN with 0.05% trifluoroacetic acid (TFA)

TABLE A1
(analytic data of the compounds of Table A):

| Compound | | | RT | | LC-MS |
|---|---|---|---|---|---|
| number | IUPAC name | STRUCTURE | (min) | [M + H] | Method |
| A1 | N-[(1- cyanocyclopropyl)methy]- 4-[(5S)-5-(3,5- dichlorophenyl)-5- (trifluoromethyl)-4H- isothiazol-3-yl]-2-methyl- benzamide | 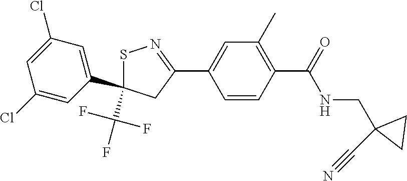 [SMILES: Cc1cc(C2=NS[C@@](c3cc(Cl)cc(Cl)c3)(C(F)(F)F)C2)ccc1C(=O)NCC1(C#N)CC1] | 1.65 | 512.3 | A |
| A2 | N-[(1- cyanocyclopropyl)methyl]- 4-[2-(3,5-dichlorophenyl)- 2-(trifluoromethyl)-3H- furan-4-yl]-2-methyl- benzamide | 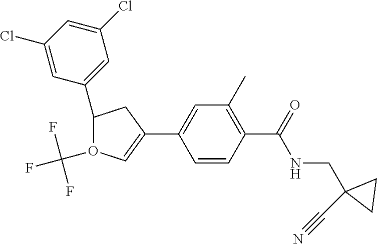 | 1.63 | 495.4 | A |
| A3 | 4-[(4S,5R)-4-chloro-5- (3,5-dichlorophenyl)-5- (trifluoromethyl)-4H- isoxazol-3-yl]-N-[(1- cyanocyclopropyl)methyl]- 2-methyl-benzamide | 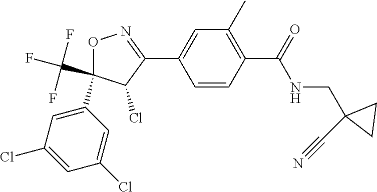 [SMILES: Cc1cc(C2=NO[C@](c3cc(Cl)cc(Cl)c3)(C(F)(F)F)[C@H]2Cl)ccc1C(=O)NCC1(C#N)CC1] | 1.64 | 530.3 | A |
| A4 | N-[(1- cyanocyclopropyl)methyl]- 4-[3-(3,5-dichloro-4- fluorophenyl)-3- (trifluoromethyl)-2,4- dihydropyrrol-5-yl]-2- methyl-benzamide | 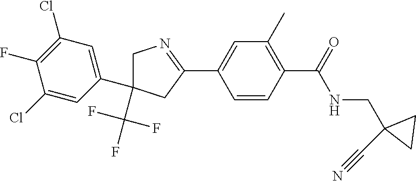 [SMILES: Cc1cc(C2=NCC(c3cc(Cl)c(F)c(Cl)c3)(C(F)(F)F)C2)ccc1C(=O)NCC1(C#N)CC1] | 1.42 | 512.4 | A |
| A5 | N-[(1- cyanocyclopropyl)methyl]- 2-methyl-4-[3-(3,4,5- trichlorophenyl)-3- (trifluoromethyl)-2,4- dihydropyrrol-5- yl]benzamide | 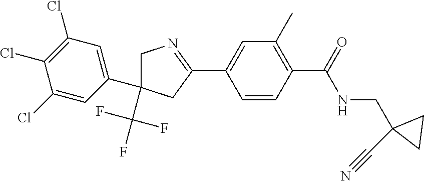 [SMILES: Cc1cc(C2=NCC(c3cc(Cl)c(Cl)c(Cl)c3)(C(F)(F)F)C2)ccc1C(=O)NCC1(C#N)CC1] | 1.49 | 528.3 | A |

### Biological Examples

These Examples illustrate the pesticidal/insecticidal properties of compounds of formula (I).

Tests were performed as follows:

### Example B1

Diabrotica balteata (Corn Root Worm)

Maize sprouts placed onto an agar layer in 24-well microtiter plates were treated with aqueous test solutions prepared from 10′000 ppm DMSO stock solutions by spraying. After drying, the plates were infested with L2 larvae (6 to 10 per well). The samples were assessed for mortality and growth inhibition in comparison to untreated samples 4 days after infestation.

The following compounds gave an effect of at least 80% in at least one of the two categories (mortality or growth inhibition) at an application rate of 200 ppm: A1, A2, A3, A4, A5

### Example B2

Euschistus heros(Neotropical Brown Stink Bug)

Soybean leaves on agar in 24-well microtiter plates were sprayed with aqueous test solutions prepared from 10′000 ppm DMSO stock solutions. After drying the leaves were infested with N2 nymphs. The samples were assessed for mortality and growth inhibition in comparison to untreated samples 5 days after infestation.

The following compounds gave an effect of at least 80% in at least one of the two categories (mortality or growth inhibition) at an application rate of 200 ppm: A1, A2, A3, A4, A5

### Example B3

### Myzus persicae (Green Peach Aphid). Intrinsic Activity

Test compounds prepared from 10′000 ppm DMSO stock solutions were applied by pipette into 24-well microtiter plates and mixed with sucrose solution. The plates were closed with a stretched Parafilm. A plastic stencil with 24 holes was placed onto the plate and infested pea seedlings were placed directly on the Parafilm. The infested plate was closed with a gel blotting paper and another plastic stencil and then turned upside down. The samples were assessed for mortality 5 days after infestation.

The following compounds resulted in at least 80% mortality at a test rate of 12 ppm: A1, A2, A3, A4, A5

### Example B4

Plutella xylostella (Diamond Back Moth)

24-well microtiter plates with artificial diet were treated with aqueous test solutions prepared from 10′000 ppm DMSO stock solutions by pipetting. After drying, the plates were infested with L2 larvae (10 to 15 per well). The samples were assessed for mortality and growth inhibition in comparison to untreated samples 5 days after infestation.

The following compounds gave an effect of at least 80% in at least one of the two categories (mortality or growth inhibition) at an application rate of 200 ppm: A1, A2, A3, A4, A5

### Example B5

### Spodoptera littoralis (Egyptian Cotton Leaf Worm)

Cotton leaf discs were placed onto agar in 24-well microtiter plates and sprayed with aqueous test solutions prepared from 10′000 ppm DMSO stock solutions. After drying the leaf discs were infested with five L1 larvae. The samples were assessed for mortality, anti-feeding effect, and growth inhibition in comparison to untreated samples 3 days after infestation. Control of Spodoptera littoralis by a test sample is given when at least one of the categories mortality, anti-feedant effect, and growth inhibition is higher than the untreated sample.

The following compounds resulted in at least 80% control at an application rate of 200 ppm: A1, A2, A3, A4, A5

### Example B6

Tetranychus urticae (Two-Spotted Spider Mite):Feeding/Contact Activity

Bean leaf discs on agar in 24-well microtiter plates were sprayed with aqueous test solutions prepared from 10′000 ppm DMSO stock solutions. After drying the leaf discs were infested with a mite population of mixed ages. The samples were assessed for mortality on mixed population (mobile stages) 8 days after infestation.

The following compounds resulted in at least 80% mortality at an application rate of 200 ppm: A1, A2, A3, A4, A5

### Example B7

Thrips tabaci (Onion Thrips) Feeding/Contact Activity

Sunflower leaf discs were placed on agar in 24-well microtiter plates and sprayed with aqueous test solutions prepared from 10′000 ppm DMSO stock solutions. After drying the leaf discs were infested with a thrips population of mixed ages. The samples were assessed for mortality 6 days after infestation.

The following compounds resulted in at least 80% mortality at an application rate of 200 ppm: A1, A2, A3, A4, A5

1. A compound of formula (I), 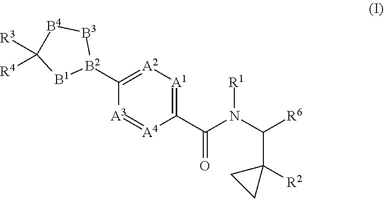
wherein
A¹, A², A³ and A⁴ are, independently of one another, C—H, C—R⁵ or N;
B¹—B²—B³—B⁴ is —C(R^{5a}R^{5b})—C═N—O—, —CH₂—C═N—CH₂—, —CH₂—C═CH₂—S—, —CH₂—C═N—S—, —CH₂—N—CH₂—CH₂—, —CH₂—C═CH—O—, —CH(OH)—N—CH₂—CH₂—, —C(O)—N—CH₂—CH₂- or —CH═C—CH₂—O—;
R¹ is hydrogen, C₁-C₈alkyl, C₁-C₈alkylcarbonyl-, C₁-C₈alkoxy, C₁-C₈alkoxy-C₁-C₈alkyl, C₁-C₈alkoxycarbonyl, C₁-C₈alkylcarbonyloxyC₁-C₈alkyl, C₁-C₈alkoxycarbonylsulfanyl, C₁-C₈alkylaminocarbonyloxyC₁-C₈alkyl, C₁-C₈dialkylaminocarbonyloxyC₁-C₈alkyl, C₁-C₈alkylaminocarbonylC₁-C₈alkyl, C₁-C₈dialkylaminocarbonylC₁-C₈alkyl or C₁-C₈alkoxycarbonylC₁-C₈alkylaminoC₁-C₈alkyl, wherein each alkyl or alkoxy group may be optionally substituted with from one to three halogen atoms or with a cyano group; or
R¹ is selected from —(C₀-C₄alkyl)-C(═O)—C₃-C₆cycloalkyl, —(C₀-C₄alkyl)-O—C(═O)—C₃-C₆cycloalkyl, —(C₀-C₄alkyl)-O—C₃-C₆cycloalkyl, —R^{1a}OR^{1a}OR^{1b}, —R^{1a}OC(═O)R^{1b}, —R^{1a}OC(═O)OR^{1b}, —R^{1a}N(R^{1c})C(═O)OR^{1b}, —R^{1a}OC(═O)N(R^{1b})(R^{1c}), —R^{1a}C(═O)N(R^{1b})(R^{1c}) and —S—C(═O)OR^{1b};
R^{1a} is —(CR^{1d}R^{1e})ₙ;
R^{1b} and R^{1c} are independently selected from H and C₁-C₄alkyl, wherein each alkyl group is unsubstituted or substituted with one to three halogen atoms or with a cyano group;
R^{1d} and R^{1e} are independently selected from H and C₁-C₄alkyl;
n is selected from 1, 2, 3 and 4;
R² is cyano, C(S)NH₂ or C(O)NH₂;
R³ is C₁-C₈haloalkyl;
R⁴ is aryl, aryl substituted by one to three R⁷, heteroaryl or heteroaryl substituted by one to three R⁷;
R⁵ is independently halogen, cyano, nitro, C₁-C₈alkyl, C₃-C₈cycloalkyl, C₁-C₈haloalkyl, C₂-C₈alkenyl, C₂-C₈haloalkenyl, C₂-C₈alkynyl, C₂-C₈haloalkynyl, C₁-C₈alkoxy, C₁-C₈haloalkoxy, or C₁-C₈alkoxycarbonyl-, or two R⁵ on adjacent carbon atoms together form a —CH═CH—CH═CH— bridge, a —CH₂—CH₂—CH₂— bridge, a —CH(OH)—CH₂—CH₂— bridge, a —C(O)—CH₂—CH₂— bridge or a —N═CH—CH═CH— bridge;
R^{5a} and R^{5b} are, independently of each other, hydrogen, cyano, halogen, hydroxyl, C₁-C₈alkyl-, C₁-C₈alkyl- substituted by one to five R^{6a}, C₁-C₈alkylthio-, C₁-C₈haloalkylthio-, C₁-C₈alkylsulfinyl-, C₁-C₈haloalkylsulfinyl-, C₁-C₈alkylsulfonyl-, C₁-C₈haloalkylsulfonyl-, arylthio- or arylthio- wherein the aryl moiety is substituted by one to five R⁷, arylsulfinyl- or arylsulfinyl- wherein the aryl moiety is substituted by one to five R⁷, arylsulfonyl- or arylsulfonyl- wherein the aryl moiety is substituted by one to five R⁷, heterocyclylthio- or heterocyclylthio- wherein the heterocyclyl moiety is substituted by one to five R⁷, heterocyclylsulfinyl- or heterocyclylsulfinyl- wherein the heterocyclyl moiety is substituted by one to five R⁷, or heterocyclylsulfonyl- or heterocyclylsulfonyl- wherein the heterocyclyl moiety is substituted by one to five R⁷, C₂-C₈alkenyl, C₂-C₈alkynyl, C₂-C₈haloalkenyl, C₁-C₈alkoxy, or C₁-C₈haloalkoxy, provided that at least one of R^{5a} and R^{5b} is not hydrogen;
R⁶ is hydrogen or C₁-C₈alkyl;
R^{6a} is independently halogen, cyano, nitro, amino, hydroxy, oxo, C₁-C₈alkylamino, hydroxyimino, C₁-C₈alkyloxyimino, di-C₁-C₈alkylamino, C₁-C₈alkoxy, acetyloxy, formyloxy, C₁-C₈haloalkoxy, C₁-C₄alkylthio or (C₁-C₄alkyl)₀₋₃silyl;
R⁷ is independently halogen, cyano, nitro, C₁-C₈alkyl, C₁-C₈haloalkyl, C₁-C₈alkoxy, C₁-C₈haloalkoxy;
or an agrochemically acceptable salt, tautomer and N-oxide thereof.

2. The compound according to claim 1, wherein A¹ is C—R⁵; A² is C—H; A³ is C—H; and A⁴ is C—H, wherein R⁵ is halogen, cyano, nitro, C₁-C₈alkyl, C₃-C₈cycloalkyl, C₁-C₈haloalkyl or C₂-C₈alkenyl.

3. The compound of formula (I) according to claim 1, wherein R² is halogen, C₁-C₄alkyl, C₃-C₅cycloalkyl, C₁-C₄haloalkyl, C₁-C₄alkoxy, C₁-C₄haloalkoxy or di-C₁-C₄alkylamino, C₁-C₄alkylthio or C₁-C₄alkyloxycarbonyl.

4. The compound of formula (I) according to claim 1, wherein R¹ is hydrogen, formyl, C₁-C₈alkyl, C₁-C₈alkylcarbonyl- or C₁-C₈alkoxycarbonyl-.

5. The compound of formula (I) according to claim 1, wherein R¹ is C₁-C₈alkyl, C₁-C₈alkylcarbonyl-, C₃-C₆cycloalkylcarbonyl, C₁-C₈alkoxy, C₁-C₈alkoxy-C₁-C₈alkyl, C₁-C₈alkoxycarbonyl, C₁-C₈alkylcarbonyloxyC₁-C₈alkyl, C₁-C₈alkoxycarbonylsulfanyl, C₁-C₈alkylaminocarbonyloxyC₁-C₈alkyl, C₁-C₈dialkylaminocarbonyloxyC₁-C₈alkyl, C₁-C₈alkylaminocarbonylC₁-C₈alkyl, C₁-C₈dialkylaminocarbonylC₁-C₈alkyl, or C₁-C₈alkoxycarbonylC₁-C₈alkylaminoC₁-C₈alkyl, wherein each alkyl or alkoxy group may be optionally substituted with from one to three halogen atoms

6. The compound of formula (I) according to claim 4, wherein R¹ is C₁-C₈cyanoalkyl, C₁-C₈alkoxy-C₁-C₈alkyl, C₁-C₈alkoxycarbonyl or C₁-C₈alkylcarbonyloxyC₁-C₈alkyl.

7. The compound of formula (I) according to claim 6, wherein R¹ is cyanomethyl, methoxymethyl, ethoxymethyl, methoxycarbonyl, ethoxycarbonyl, methylcarbonyloxymethyl, 1-methylethylcarbonyloxymethyl or 1,1-dimethylethylcarbonyloxymethyl.

8. The compound according to claim 1, wherein R³ is C₁-C₄haloalkyl.

9. The compound according to claim 1, wherein R⁴ is phenyl or phenyl substituted by one to three R^{6b}; R^{6b} independently is R^{6b} independently is halogen, cyano, C₁-C₄alkyl, C₁-C₄haloalkyl, C₁-C₄alkoxy, or C₁-C₄haloalkoxy.

10. The compound according to claim 1, wherein R^{5a} is halogen, hydroxyl, C₁-C₈alkylthio-, C₁-C₈haloalkylthio-, C₁-C₈alkylsulfinyl-, C₁-C₈haloalkylsulfinyl-, C₁-C₈alkylsulfonyl-, C₁-C₈haloalkylsulfonyl-, C₁-C₈alkyl, C₂-C₈alkenyl, C₂-C₈alkynyl, C₁-C₈haloalkyl, C₂-C₈haloalkenyl; R^{5b} is halogen or hydrogen.

11. The compound according to claim 1, wherein R⁵ is halogen, cyano, C₁-C₈alkyl, C₂-C₈ alkenyl, C₃-C₈cycloalkyl, or C₁-C₈haloalkyl.

12. A pesticidal composition, which comprises at least one compound of formula (I) according to claim 1 or, where appropriate, a tautomer thereof, in each case in free form or in agrochemically utilizable salt form, as active ingredient.

13. A method for controlling pests, which comprises applying a composition according to claim 13 to the pests or their environment with the exception of a method for treatment of the human or animal body by surgery or therapy and diagnostic methods practised on the human or animal body.

14. A method for the protection of plant propagation material from the attack by pests, which comprises treating the propagation material or the site, where the propagation material is planted, with a composition according to claim 13.

15. A plant propagation material treated with the pesticidal composition of claim 12.
